(19) United States
(12) Patent Application Publication
(10) Pub. No.: US 2025/0197377 A1
(43) Pub. Date: Jun. 19, 2025

(54) NOVEL PHARMACEUTICAL SALTS AND POLYMORPHIC FORMS OF AN ERBB AND BTK INHIBITOR

(71) Applicant: DIZAL (JIANGSU) PHARMACEUTICAL CO., LTD., Shanghai (CN)

(72) Inventors: Jun-Cheng ZHENG, Shanghai (CN); Jianan JIANG, Shanghai (CN); Qinghai GUO, Shanghai (CN); Shih-Ying CHANG, Shanghai (CN); Qingbei ZENG, Shanghai (CN); Honchung TSUI, Shanghai (CN); Zhenfan YANG, Shanghai (CN); Xiaolin ZHANG, Shanghai (CN)

(21) Appl. No.: 18/293,778

(22) PCT Filed: Jul. 29, 2022
(86) PCT No.: PCT/CN2022/109065

§ 371 Date: Jan. 31, 2024

(30) Foreign Application Priority Data
Aug. 2, 2021 (WO) PCT/CN2021/110048

(51) Int. Cl. C07D 403/12; A61K 31/506; A61K 45/06

(52) U.S. Cl. CPC C07D 403/12; A61K 31/506; A61K 45/06

(57) ABSTRACT

Disclosed are novel pharmaceutical salts and polymorphic forms of (R)—N-(5-((4-((5-chloro-4-fluoro-2-(2-hydroxypropan-2-yl)phenyl)amino)pyrimidin-2-yl)amino)-2-(3-(dimethylamino)pyrrolidin-1-yl)-4-methoxyphenyl)acrylamide (Compound I) that has inhibitory activities against ErbBs (e.g. EGFR or Her2) and/or BTK, especially mutant forms of ErbBs, and/or BTK. Further disclosed herein are the processes for the preparation of pharmaceutical salts and polymorphic forms of Compound I, and uses of such pharmaceutical salts and polymorphic forms of Compound I in inhibiting the ErbB or BTK.

### FIELD OF THE DISCLOSURE

The present disclosure relates to novel pharmaceutical salts of ((R)—N-(5-((4-((5-chloro-4-fluoro-2-(2-hydroxypropan-2-yl)phenyl)amino)pyrimidin-2-yl)amino)-2-(3-(dimethylamino)pyrrolidin-1-yl)-4-methoxyphenyl)acrylamide (hereinafter, “Compound I”, having a structure as shown below)):

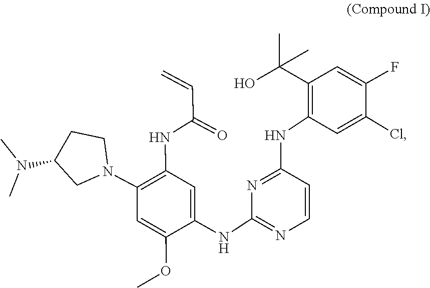 [SMILES: C=CC(=O)Nc1cc(Nc2nccc(Nc3cc(Cl)c(F)cc3C(C)(C)O)n2)c(OC)cc1N1CC[C@@H](N(C)C)C1]

crystalline polymorphs of Compound I or the pharmaceutical salts, composition comprising the same, the preparation process and uses thereof.

### TECHNICAL BACKGROUND

ErbB family receptor tyrosine kinases act to transmit signals from the outside of a cell to the inside by activating secondary messaging effectors via a phosphorylation event at their tyrosine phosphorylation residues. A variety of cellular processes are modulated by these signals, including proliferation, carbohydrate utilization, protein synthesis, angiogenesis, cell growth, and cell survival. Deregulation of ErbB family signalling modulates proliferation, invasion, metastasis, angiogenesis, and tumour cell survival and may be associated with many human cancers, including those of the lung, head and neck and breast cancers. Various ErbB receptors such as EGFR, and HER2 have been demonstrated to relate to disorders such as cancer. Different mutations of EGFR and HER2 have also been proved to relate to certain cancer type or to the non-responsiveness/resistance to existing drugs for WT EGFR or HER2.

Bruton's tyrosine kinase (BTK) is a member of the SRC-related family of cytoplasmic tyrosine kinases, which are predominantly expressed in B cells, and distributed in the lymphatic system, hematopoietic and hematological systems. BTK plays a key role in the B-cell receptor signaling pathway of B-cells, which is required for the development, activation and survival of B-cells. BTK inhibitors have therefore been developed with the aim of treating B-cell malignancies that are dependent on BCR signaling, such as chronic lymphocytic leukemia (CLL) and non-Hodgkin's lymphoma (NHL), mantle cell lymphoma (MCL), and diffuse large B-cell lymphoma (DLBCL). BTK has also been implicated in promotion of Toll-like receptor signaling, which regulates macrophage activation and production of proinflammatory cytokines. Several studies have demonstrated crosstalk between BTK and TLR signaling pathways to mediate transactivation of downstream cascades. Furthermore, BTK is found to play a critical role in regulation of immunity. BTK has become an attractive target for the treatment of B-cell malignancies, inflammation, as well as the treatment of autoimmune diseases.

Crystalline polymorphs are different crystalline forms of the same compound. Different crystalline polymorphs may have different crystal structures due to a different packing of the molecules in the lattice. This results in a different crystal symmetry and/or unit cell parameters which directly influences its physical properties such as the X-ray diffraction characteristics of crystals or powders. A different polymorph, for example, will in general diffract at a different set of angles and will give different values for the intensities. Therefore, X-ray powder diffraction can be used to identify different polymorphs, or a solid form that comprises more than one polymorph, in a reproducible and reliable way.

Crystalline polymorphic forms are of interest to the pharmaceutical industry and especially to those involved in the development of suitable dosage forms. Different crystalline forms of a drug substance can have different physical properties, including melting point, solubility, dissolution rate, optical and mechanical properties, vapor pressure, hygroscopicity, particle shape, density, and flowability. These properties can have a direct effect on the ability to process and/or manufacture a compound as a drug product. Different crystalline forms can also exhibit different stabilities and bioavailability. For example, if the polymorphic form is not held constant during clinical or stability studies, the exact dosage form used or studied may not be comparable from one lot to another. Therefore, the most stable crystalline form of a drug product is often chosen during drug development based on the minimal potential for conversion to another crystalline form and on its greater chemical stability. It is also desirable to have processes for producing a compound with the selected polymorphic form in high purity when the compound is used in clinical studies or commercial products since impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies. To ensure the quality, safety, and efficacy of a drug product, it is important to choose a crystalline form that is stable, is manufactured reproducibly, and has favorable physicochemical properties.

In WO2019149164A1 (the disclosure of which is hereby incorporated in its entirety) we have described various ErbB/BTK-selective inhibitors, including (R)—N-(5-((4-((5-chloro-4-fluoro-2-(2-hydroxypropan-2-yl)phenyl)amino)pyrimidin-2-yl) amino)-2-(3-(dimethylamino)pyrrolidin-1-yl)-4-methoxyphenyl)acrylamide (Compound I) which has been proven as a potent ErbB/BTK-selective inhibitor. It is of interest to identify crystalline polymorphic forms of this compound or its pharmaceutical salts for further development of pharmaceutical composition or dosage forms.

The synthetic methods of Compound I known in the art are not suitable for the large-scale, particularly commercial scale manufacturing of Compound I. An improved process that can be operated at large scale and provide one or more advantages relative to the known methods, such as improved compound purity, improved compound isolation, higher yield, reduced cost, improved compliance with regulatory requirements for pharmaceutical starting materials, intermediates, and products are in need.

### SUMMARY

In one aspect, the present disclosure provides novel pharmaceutical salts of Compound I.

In some embodiments, the pharmaceutical salt of Compound I provided herein is selected from: hydrochloric acid salt, methanesulfonic acid salt, sulfuric acid salt, phosphoric acid salt, maleic acid salt, fumaric acid salt, citric acid salt, succinic acid salt, L-malic acid salt, L-(+)-tartaric acid salt, and hydrochloric acid salt of Compound I. In certain embodiments, the pharmaceutical salt of Compound I is hydrochloric acid salt, L-(+)-tartaric acid salt, fumaric acid salt, sulfuric acid salt, and maleic acid salt of Compound I. In certain embodiments, the pharmaceutical salt of Compound I is in amorphous form. In certain embodiments, the pharmaceutical salt of Compound I is in crystalline form. In certain embodiments, the pharmaceutical salt of Compound I is hydrochloric acid salt, L-(+)-tartaric acid salt, fumaric acid salt, sulfuric acid salt, and maleic acid salt of Compound I in crystalline form.

In another aspect, the present disclosure also provides crystalline form of Compound I or pharmaceutically acceptable salts thereof.

In some embodiments, the crystalline form is Form A of Compound I, Form B of Compound I, crystalline form of hydrochloric acid salt, L-(+)-tartaric acid salt, fumaric acid salt, sulfuric acid salt, or maleic acid salt of Compound I.

In another aspect, the present disclosure provides pharmaceutical compositions, each comprising one or more pharmaceutical salts or crystalline forms of Compound I, as disclosed herein.

In another aspect, the present disclosure provides methods of treating an ErbB associated disease or BTK associated disease in a subject, comprising administering to a subject in need thereof a therapeutically effective amount of the pharmaceutical salts or crystalline forms of Compound I, or pharmaceutical composition provided herein.

In yet another aspect, the present disclosure provides use of the pharmaceutical salts or crystalline forms of Compound I, or pharmaceutical composition provided herein in inhibiting ErbB or BTK, or in the manufacture of a medicament for inhibiting ErbB or BTK.

In a further aspect, the present disclosure also provides process for production of the pharmaceutical salts or the crystalline form of Compound I.

In a further aspect, the present disclosure also provides process for preparing Compound I on tens of kilogram scale with high yield.

### DESCRIPTION OF THE DRAWINGS

FIG. 1 is the XRPD data for the crystalline Form A of the free base of Compound I.

FIG. 2 is the DSC data for the crystalline Form A of the free base of Compound I.

FIG. 3 is the TGA data for the crystalline Form A of the free base of Compound I.

FIG. 4 is the DVS data for the crystalline Form A of the free base of Compound I.

FIG. 5 is the XRPD data for the crystalline Form B of the free base of Compound I.

FIG. 6 is the DSC data for the crystalline Form B of the free base of Compound I.

FIG. 7 is the TGA data for the crystalline Form B of the free base of Compound I.

FIG. 8 is the DVS data for the crystalline Form B of the free base of Compound I.

FIG. 9 is the XRPD data for the (+)-L-tartaric acid salt of Compound I (pattern I).

FIG. 10 is the XRPD data for the fumaric acid salt of Compound I.

FIG. 11 is the XRPD data for the sulfuric acid salt of Compound I.

FIG. 12 is the XRPD data for the maleic acid salt of Compound I.

FIG. 13 is the XRPD data for the hydrochloric acid salt of Compound I.

FIG. 14 is the XRPD data for the (+)-L-tartaric acid salt of Compound I (pattern II).

FIG. 15 is the TGA/DSC overlay data for the (+)-L-tartaric acid salt of Compound I (pattern I, prepared in acetone).

FIG. 16 is the TGA/DSC overlay data for the (+)-L-tartaric acid salt of Compound I (pattern II, prepared in ethanol).

FIG. 17 is the TGA/DSC overlay data for the fumaric acid salt of Compound I (prepared in ethanol).

FIG. 18 is the TGA/DSC overlay data for the sulfuric acid salt of Compound I.

FIG. 19 is the TGA/DSC overlay data for the maleic acid salt of Compound I.

FIG. 20 is the TGA/DSC overlay data for the hydrochloric acid salt of Compound I.

FIG. 21 is the DVS data for crystalline form of the Compound I-(+)-L-tartaric acid salt. (pattern II).

FIG. 22 is the DVS data for crystalline form of the Compound I-fumaric acid salt.

FIG. 23 is the DVS data for crystalline form of the Compound I-hydrochloric acid salt.

FIG. 24 is the XRPD profiles of Compound I-Form B before and after jet milling.

FIG. 25 is the XRPD profiles of Compound I-Form B before and after grinding.

FIG. 26 is the DSC profiles of Compound I-Form B before and after jet milling.

FIG. 27 is the XRPD profiles of Compound I-Form B after storage for 20 days at 2-8° C.

FIG. 28 is the DSC profiles of Compound I-Form B after storage for 20 days at 2-8° C.

FIG. 29 is the ¹H NMR for the determination of Compound I-fumaric acid salt ratio (1:1).

FIG. 30 is the ¹H NMR for the determination of Compound I-maleic acid salt ratio (1:1).

FIG. 31 is the ¹H NMR for the determination of Compound I-tartaric acid salt (pattern I) ratio (1:1).

FIG. 32 is the ¹H NMR for the determination of Compound I-tartaric acid salt (pattern II) ratio (1:1).

FIG. 33 is the single crystal X-ray diffraction ORTEP of Compound I.

FIG. 34 is the Dissolution profile of 200 mg tablets for Compound I at pH1.2.

FIG. 35 is the Dissolution profile of 200 mg tablets for Compound I at pH4.5.

### DETAILED DESCRIPTION

Prior to discussing in further detail, the following terms will be defined.

### Definitions

Unless defined otherwise, all technical and scientific terms used herein have the same meaning as commonly understood by one of ordinary skill in the art to which this invention belongs. As used herein, the following terms are intended to have the following meanings:

As used in the specification and claims, the singular forms “a”, “an”, and “the” and the like includes plural references unless the context clearly dictates otherwise. Thus, for example, reference to “a compound” includes both a single compound and a plurality of different compounds.

The term “about” as used herein intends to indicate that the values quoted are not to be construed as absolute, and the measurement error, inter-batches variation and/or inter-apparatus variations as described above should also be taken into account. Except for where the range of measurement error or variation is specified in this application (e.g. the measurement error is ±0.2° for the diffraction angle 20 in XRPD, the measurement error is ±0.01-10° C. of the endotherms for crystal polymorph melting and ±0.01-20° C. of the endotherms for polymorph dehydration/desolvation in DSC, the measurement error is ±5-20° C. in TGA), the term “about” when used before a numerical designation, e.g., temperature, time, amount, and concentration, including a range, indicates approximations which may vary by ±10%, ±5% or ±10%.

As used herein, “inhibitor” refers to a compound or agent having the ability to inhibit a biological function of a target protein or polypeptide, such as by inhibiting the activity or expression of the target protein or polypeptide. Accordingly, the term “inhibitor” is defined in the context of the biological role of the target protein or polypeptide. While some inhibitors herein specifically interact with (e.g., bind to) the target, compounds that inhibit a biological activity of the target protein or polypeptide by interacting with other members of the signal transduction pathway of that target protein or polypeptide are also specifically included within this definition. Non-limiting examples of biological activity inhibited by an inhibitor include those associated with the development, growth, or spread of a tumor, or an undesired immune response as manifested in autoimmune disease. As used herein, “selective inhibition” or “selectively inhibit” as applied to a biologically active agent refers to the agent's ability to selectively reduce the target signaling activity as compared to off-target signaling activity, via direct or indirect interaction with the target. For example, a compound that selectively inhibits mutant EGFR/Her2 over wild-type EGFR/Her2 has an activity of at least about 2× against the mutated EGFR/Her2 relative to the compound's activity against the wild-type EGFR/Her2 isoform (e.g., at least about 3×, about 5×, about 10×, about 20×, about 50×, or about 100×).

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments, compounds, materials, compositions, and/or dosage forms that are pharmaceutically acceptable refer to those approved by a regulatory agency (such as U.S. Food and Drug Administration, China Food and Drug Administration or European Medicines Agency) or listed in generally recognized pharmacopoeia (such as U.S. Pharmacopoeia, China Pharmacopoeia or European Pharmacopoeia) for use in animals, and more particularly in humans.

As used herein, “pharmaceutically acceptable salts” or “pharmaceutical salts” refers to derivatives of the compounds of present disclosure wherein the parent compound is modified by converting an existing acidic moiety (e.g., carboxyl and the like) or base moiety (e.g., amine, alkali and the like) to its salt form. In many cases, compounds of present disclosure are capable of forming acid addition salts and/or base salts by virtue of the presence of amino, alkali and/or carboxyl groups or groups similar thereto. And the “pharmaceutically acceptable salt” includes acid addition salts or base salts that retain biological effectiveness and properties of the parent compound, which typically are not biologically or otherwise undesirable. Pharmaceutically acceptable salts are well known in the art. For example, Berge et al. describes pharmaceutically acceptable salts in detail in J. Pharmaceutical Sciences (1977) 66: 1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, propionic acid, glycolic acid, pyruvic acid, oxalic acid, lactic acid, trifluoracetic acid, benzoic acid, cinnamic acid, mandelic acid, ethanesulfonic acid, p-toluenesulfonic acid, salicylic acid, malonic acid, fumaric acid, citric acid, malic acid, maleic acid, tartaric acid, succinic acid, or methanesulfonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, methanesulfonic acid, maleic acid, fumaric acid, citric acid, succinic acid, L-malic acid, L-(+)-tartaric acid, and the like. In certain embodiments, the pharmaceutically acceptable salt is a hydrochloric acid salt, methanesulfonic acid salt, sulfuric acid salt, phosphoric acid salt, maleic acid salt, fumaric acid salt, citric acid salt, succinic acid salt, L-malic acid salt, and L-(+)-tartaric acid salt.

As used herein, the term “polymorphic form”, “polymorph” or “crystalline form” refers to a solid in which the constituent atoms, molecules, or ions are packed in a regularly ordered, repeating three-dimensional pattern having a highly regular chemical structure. In particular, a compound or salts thereof might be produced in one or more crystalline forms. Different crystalline forms can be characterized by XRPD patterns (e.g. X-ray diffraction peaks position at various diffraction angles (20) and/or peak intensities), melting point onset (and onset of dehydration for hydrated forms) as illustrated by endotherms of a differential scanning calorimetry (DSC) thermogram, thermal gravimetric analysis (TGA), solid state ¹H nuclear magnetic resonance (NMR) spectrum, aqueous solubility, high intensity light conditions, physical and chemical storage stability and any other measurements known in the art.

An “XRPD pattern” refers to the experimentally observed diffractogram or parameters derived therefrom, which is shown as an x-y graph with peak positions represented as diffraction angle (20) on the x-axis and peak intensity on the y-axis. The peaks within this pattern may be used to characterize a crystalline solid form.

The term “peak positions” as used herein refers to X-ray reflection positions as measured and observed in X-ray powder diffraction experiments. Peak positions are directly related to the dimensions of the unit cell. The peaks, identified by their respective peak positions, have been extracted from the diffraction patterns for the various polymorphic forms of Compound I disclosed herein.

The term “peak intensities” refers to relative signal intensities within a given X-ray powder diffraction pattern. Factors that can affect the relative peak intensities are sample thickness and preferred orientation (i.e., the crystalline particles are not distributed randomly).

As with any data measurement, there is variability in XRPD data. The data are often represented solely by the diffraction angle of the peaks rather than including the intensity of the peaks because peak intensity can be particularly sensitive to sample preparation (for example, particle size, moisture content, solvent content, and preferred orientation effects influence the sensitivity), so samples of the same material prepared under different conditions may yield slightly different patterns; this variability is usually greater than the variability in diffraction angles. Diffraction angle variability may also be sensitive to sample preparation. Other sources of variability come from instrument parameters and processing of the raw X-ray data: different X-ray instruments operate using different parameters and these may lead to slightly different XRPD patterns from the same solid form, and similarly different software packages process X-ray data differently and this also leads to variability. These and other sources of variability are known to those of ordinary skill in the pharmaceutical arts. Due to such sources of variability, a measurement error of a diffraction angle in an XRPD is approximately 20 (±0.2°), and such degree of a measurement error should be taken into account when considering the XRPD pattern in the Figures and when reading data contained in the Tables included herein.

DSC measures the difference in heat energy between a solid sample and an appropriate reference with an increase in temperature. DSC thermograms are characterized by endotherms (indicating energy uptake) and also by exotherms (indicating energy release), typically as the sample is heated. A person skilled in the art also understands that the value or range of values observed in a particular compound's DSC thermogram will show variation between batches of different purities. Depending upon the rate of heating (i.e., the scan rate) at which the DSC analysis is conducted, the way the DSC on-set temperature is defined and determined, the calibration standard used, the instrument calibration, and the relative humidity (RH) and chemical purity of the sample, the endotherms exhibited by the compounds of the present disclosure may vary (±0.01-10° C. of the endotherms for crystal polymorph melting and ±0.01-20° C. of the endotherms for polymorph dehydration/desolvation), and such degree of variation should be taken into account when considering the DSC data included herein. To further clarify, one compound prepared in different batches may show variations in DSC thermograms, however these DSC thermograms with variations should still be considered as “substantially similar to” each other. For any given example, the observed endotherms may also differ from instrument to instrument; however, it will generally be within the ranges defined herein provided the instruments are calibrated similarly. Furthermore, it will be understood that removal of the residual solvent in the prepared compounds may also change the DSC onset and peak temperatures.

TGA is a testing procedure in which changes in weight of a specimen are recorded as the specimen is heated in air or in a controlled atmosphere such as nitrogen. Thermogravimetric curves (thermograms) provide information regarding solvent and water content and the thermal stability of materials. TGA thermograms show similar variations as DSC (a measurement error of about ±5-20° C.), such that a person skilled in the art recognizes that measurement errors should be taken into account when judging substantial identity of TGA thermograms.

It is to be understood that the “compound” of present disclosure can exist in solvated as well as un-solvated forms, such as, for example, hydrated forms, solid forms, and the present disclosure is intended to encompass all such solvated and unsolvated forms. It is further to be understood that the “compound” of present disclosure can exist in forms of pharmaceutically acceptable salts or esters.

Unless otherwise specified, “ErbB” or “wild-type ErbB” refers to normal ErbB family members. In one aspect, the present disclosure provides inhibitory compounds of ErbB family kinase (e.g., EGFR, Her2, Her3 and/or Her4). In some embodiments, the compounds of the present disclosure can inhibit both Wild-Type (WT) and mutant forms of ErbB family kinase. In some embodiments, the compounds of the present disclosure are selective inhibitors of at least one mutation of ErbB family kinase as compared to corresponding WT ErbB family kinase.

As used herein, the term “mutations” refers to the any mutations to the target protein, “mutant” or “mutated form” refers to the protein that contains said mutation. Exemplary mutations of ErbBs, include but are not limited to, EGFR D761_E762insEAFQ, EGFR A763_Y764insHH, EGFR M766_A767instAI, EGFR A767_V769dupASV, EGFR A767_S768insTLA, EGFR S768_D770 dupSVD, EGFR S768_V769insVAS, EGFR S768_V769insAWT, EGFR V769_D770insASV, EGFR V769_D770insGV, EGFR V769_D770insCV, EGFR V769_D770insDNV, EGFR V769_D770insGSV, EGFR V769_D770insGVV, EGFR V769_D770insMASVD, EGFR D770 N771insSVD, EGFR D770 N771insNPG, EGFR D770 N771insAPW, EGFR D770_N771insD, EGFR D770_N771insDG, EGFR D770_N771insG, EGFR D770_N771insGL, EGFR D770_N771insN, EGFR D770_N771insNPH, EGFR D770_N771insSVP, EGFR D770_N771insSVQ, EGFR D770_N771insMATP, EGFR delD770insGY, EGFR N771_P772insH, EGFR N771_P772insN, EGFR N771_H773dupNPH, EGFR delN771insGY, EGFR delN771insGF, EGFR P772_H773insPR, EGFR P772_H773insYNP, EGFR P772_H773insX, EGFR P772_H773insDPH, EGFR P772_H773insDNP, EGFR P772_H773insQV, EGFR P772 H773insTPH, EGFR P772 H773insN, EGFR P772 H773insV, EGFR H773 V774insNPH, EGFR H773 V774insH, EGFR H773 V774insPH, EGFR H773 V774insGNPH, EGFR H773 V774dupHV, EGFR H773_V774insG, EGFR H773 V774insGH, EGFR V774_C775insHV, EGFR exon19 deletion, EGFR L858R, EGFR T790M, EGFR L858R/T790M, EGFR exon 19 deletion/T790M, EGFR S768I, EGFR G719S, EGFR G719A, EGFR G719C, EGFR E709A/G719S, EGFR E709A/G719A, EGFR E709A/G719C, EGFR L861Q and the like in EGFR; and Her2 A775_G776insYVMA, Her2 delG776insVC, Her2 V777_G778insCG, Her2 P780_Y781insGSP and the like in Her2. In some embodiments, the compounds of the present disclosure are selective inhibitors of at least one mutation of EGFR as compared to WT EGFR. In some embodiments, the compounds of the present disclosure are selective inhibitors of at least one mutation of Her2 as compared to WT Her2. In some embodiments, the at least one mutation of EGFR is a point mutation (e.g., L858R, T790M). In some embodiments, the at least one mutation of EGFR is a deletion mutation (e.g., delE746-A750). In some embodiments, the at least one mutation of EGFR is an insertion mutation (e.g., EGFR Exon 20 V769_D770insASV, Exon 20 H773 V774insNPH). In some embodiments, the at least one mutation of EGFR is an activating mutation (e.g., L858R, G719S or delE746-A750). In some embodiments, the at least one mutation of EGFR is a drug resistant mutation (e.g., Exon 20_T790M). In certain embodiments, an at least one mutation of EGFR is T790M. In some embodiments, a provided compound selectively inhibits T790M/L858R co-mutation, and is sparing as to WT EGFR inhibition.

As used herein, the term “selectively inhibits,” as used in comparison to inhibition of WT EGFR/Her2, means that a provided compound is more potent as an inhibitor of at least one mutation of EGFR/Her2 (i.e., at least one point mutation, at least one deletion mutation, at least one insertion mutation, at least one activating mutation, at least one resistant mutation, or a combination of at least one deletion mutation and at least one point mutation) in at least one assay described herein (e.g., biochemical or cellular). In some embodiments, the term “selectively inhibits,” as used in comparison to WT EGFR/Her2 inhibition means that a provided compound is at least 100 times more potent, at least 50 times, at least 45 times, at least 40 times, at least 35 times, at least 30 times, at least 25 times, at least 20 times, at least 15 times, at least 10 times, at least 5 times, at least 4 times, at least 3 times, at least 2 times, at least 1.5 times, or at least 1.25 times more potent as an inhibitor of at least one mutation of EGFR/Her2, as defined and described herein, as compared to WT EGFR/Her2. In some embodiments, the term “selectively inhibits,” as used in comparison to WT EGFR/Her2 inhibition means that a provided compound is up to 1500 times more potent, up to 1200 times, up to 1000 times, up to 800 times, up to 600 times, up to 400 times, up to 200 times, up to 100 times, up to 50 times, up to 10 times more potent as an inhibitor of at least one mutation of EGFR/Her2, as defined and described herein, as compared to WT EGFR/Her2. As used herein, the term “sparing as to WT EGFR/Her2” means that said selective inhibitor of at least one mutation of EGFR/Her2, as defined and described above and herein, cannot inhibits WT EGFR/Her2 within the upper limit of detection of at least one assay as described herein (e.g., biochemical or cellular as described in detail in Examples). In some embodiments, the term “sparing as to WT EGFR/Her2” means that a provided compound inhibits WT EGFR/Her2 with an IC₅₀ of at least 10 μM, at least 9 μM, at least 8 μM, at least 7 μM, at least 6 μM, at least 5 μM, at least 3 μM, at least 2 μM, or at least 1 μM. In some embodiments, compounds of the present disclosure inhibit phosphorylation of WT EGFR/Her2 and/or mutant EGFR/Her2 with an IC₅₀ value of 0.1-1000 nM, preferably 0.1-600 nM, 1-600 nM, 0.1-500 nM, 1-500 nM, 0.1-400 nM, 1-400 nM, 0.1-300 nM, 1-300 nM, 0.1-200 nM, 1-200 nM, 0.1-100 nM, 1-100 nM, 0.1-80 nM, 0.1-50 nM, 0.1-40 nM, 0.1-30 nM, 0.1-20nmM, 0.1-10 nM, or 0.1-5 nM, more preferably 0.1-20 nM, 0.1-10 nM, or 0.1-5 nM. In some embodiments, compounds of the present disclosure inhibit proliferation of WT EGFR/Her2 and/or mutant EGFR/Her2 bearing cells with an GI₅₀ value of 1-1000 nM, preferably 1-800 nM, 1-600 nM, 1-500 nM, 1-400 nM, 1-300 nM, 1-300 nM, 1-200 nM, 1-100 nM, 1-80 nM, 1-60 nM, 1-40 nM, 1-20 nM, or 1-10 nM more preferably 1-300 nM, 1-200 nM, 1-100 nM, 1-80 nM, 1-60 nM, 1-40 nM, 1-20 nM, or 1-10 nM. In some embodiments, compounds of the present disclosure inhibit proliferation of BTK bearing cells with an GI₅₀ value of 1-1000 nM, more than 1000 nM, more than 2000 nM, or more than 3000 nM preferably 1-800 nM, 1-600 nM, 1-500 nM, 1-400 nM, 1-300 nM, 1-300 nM, 1-200 nM, 1-100 nM, 1-80 nM, 1-60 nM, 1-40 nM, 1-20 nM, or 1-10 nM more preferably 1-300 nM, 1-200 nM, 1-100 nM, 1-80 nM, 1-60 nM, 1-40 nM, 1-20 nM, or 1-10 nM. In some embodiments, the IC₅₀ and/or GI₅₀ of the compounds to EGFR/Her2 mutant is at least 2 times, 3 times, 4 times, 5 times, preferably 10 times, 20 times, 30 times, 50 times, or 100 times higher than the IC₅₀ and/or GI₅₀ of the compounds to wild-type EGFR/Her2.

The term “pharmaceutical composition” refers to a mixture of one or more physiologically/pharmaceutically acceptable salts of Compound I described herein or polymorphs of Compound I or the salts, with other chemical components, such as physiologically/pharmaceutically acceptable diluent, excipient or carrier. The purpose of a pharmaceutical composition is to facilitate administration of a compound to a subject.

As used herein, the term “sustained released form” refers to release of the active agent from the pharmaceutical composition so that it becomes available for bio-absorption in the subject, primarily in the gastrointestinal tract of the subject, over a prolonged period of time (extended release), or at a certain location (controlled release).

The term “pharmaceutically acceptable carrier” as used herein refers to a pharmaceutically-acceptable material, composition or vehicle, such as a liquid or solid filler, diluent, excipient, solvent or encapsulating material, involved in carrying or transporting a compound provided herein from one location, body fluid, tissue, organ (interior or exterior), or portion of the body, to another location, body fluid, tissue, organ, or portion of the body. Pharmaceutically acceptable carriers can be vehicles, diluents, excipients, or other materials that can be used to contact the tissues of an animal without excessive toxicity or adverse effects. Non-limiting examples of pharmaceutically acceptable carriers include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-α-tocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of compounds described herein. Pharmaceutically acceptable carrier that can be employed in present disclosure includes those generally known in the art, such as those disclosed in “Remington Pharmaceutical Sciences” Mack Pub. Co., New Jersey (1991), which is incorporated herein by reference.

As used herein, “administration” of a disclosed compound encompasses the delivery to a subject of a compound as described herein, or a prodrug or other pharmaceutically acceptable derivative thereof, using any suitable formulation or route of administration, as discussed herein.

The term “effective amount” or “therapeutically effective amount” refers to the amount of a compound or pharmaceutical composition described herein that is sufficient to prevent, treat, reduce and/or ameliorate the symptoms and/or underlying causes of any disorder or disease in a subject, or the amount of an agent sufficient to produce a desired effect on target cells, e.g., reduction of cell migration. In one embodiment, a “therapeutically effective amount” is an amount sufficient to reduce or eliminate a symptom of a disease. In another embodiment, a therapeutically effective amount is an amount sufficient to overcome the disease itself. In certain specific embodiments, a “therapeutically effective amount” is an amount effective for detectable killing or inhibition of the growth or spread of cancer cells, reducing in the size or number of tumors; or other measure of the level, stage, progression or severity of the cancer. The therapeutically effective amount will vary depending upon the subject and the condition being treated, the weight and age of the subject, the severity of the condition, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. The specific dose will vary depending on, for example, the particular compounds chosen, the species of the subject and their age/existing health conditions or risk for health conditions, the dosing regimen to be followed, the severity of the disease, whether it is administered in combination with other agents, timing of administration, the tissue to which it is administered, and the physical delivery system in which it is carried. Thus, a therapeutically effective amount may be administered in one or more administrations. For example, and without limitation, a therapeutically effective amount of an agent, in the context of treating cancer, refers to an amount of the agent that alleviates, ameliorates, palliates, or eliminates one or more symptoms of cancer in the patient.

As used herein, the term “diseases associated with BTK” or “BTK associated diseases” refers to diseases whose onset or development or both are associated with the genomic alterations or mutation, expression or activity of BTK.

As used herein, the term “diseases associated with ErbB” or “ErbB associated diseases” refers to diseases whose onset or development or both are associated with the genomic alterations or mutation, expression or activity of ErbB (including EGFR and Her2). Examples of “diseases associated with ErbB” include “diseases associated with EGFR” or “diseases associated with Her2”. The term “diseases associated with EGFR” or “EGFR associated diseases” or “diseases associated with Her2” or “Her2 associated diseases” refers to diseases whose onset or development or both are associated with the genomic alterations or mutation, expression or activity of EGFR or Her2, as the case may be. Examples include but are not limited to, immune-related diseases, proliferative disorders, cancer, and other diseases.

As used herein, the terms “treatment”, “treat” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to present or delay their recurrence.

As used herein, “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, rabbits, hamsters, mice, cats, and/or dogs; and/or birds, including commercially relevant birds such as chickens, ducks, geese, quail, and/or turkeys.

### Compound I

The compound ((R)—N-(5-((4-((5-chloro-4-fluoro-2-(2-hydroxypropan-2-yl)phenyl)amino)pyrimidin-2-yl)amino)-2-(3-(dimethylamino)pyrrolidin-1-yl)-4-methoxyphenyl)acrylamide (Compound I) described in WO2019149164A1 is a potent ErbB inhibitor and BTK inhibitor which has the following structure:

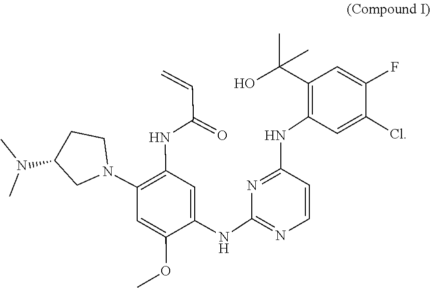 [SMILES: C=CC(=O)Nc1cc(Nc2nccc(Nc3cc(Cl)c(F)cc3C(C)(C)O)n2)c(OC)cc1N1CC[C@@H](N(C)C)C1]

Provided herein are novel pharmaceutical salts of Compound I, crystalline polymorphs of Compound I or the pharmaceutical salts of the present disclosure, composition thereof, process for production of the same and the uses thereof such as inhibiting ErbB or BTK, treating an ErbB associated diseases or BTK associated diseases in a subject.

### Pharmaceutical Salts of Compound I

In one aspect, the present disclosure provides novel pharmaceutical salts of Compound I.

In some embodiments, the pharmaceutical salt of Compound I provided herein is selected from: hydrochloric acid salt, methanesulfonic acid salt, sulfuric acid salt, phosphoric acid salt, maleic acid salt, fumaric acid salt, citric acid salt, succinic acid salt, L-malic acid salt, L-(+)-tartaric acid salt, and hydrochloric acid salt of Compound I.

In some embodiments, pharmaceutical salt of Compound I is a compound having the structure of Formula (I):

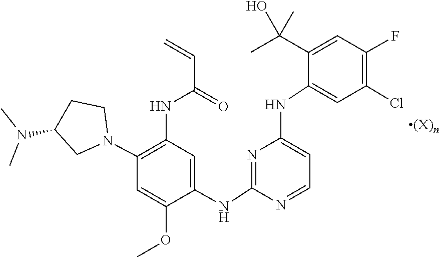

wherein, n=1 or 2; and
X is hydrochloric acid, L-(+)-tartaric acid, fumaric acid, sulfuric acid, or maleic acid.

In certain embodiments, pharmaceutical salt of Compound I is hydrochloric acid salt, L-(+)-tartaric acid salt, fumaric acid salt, sulfuric acid salt, and maleic acid salt of Compound I. In certain embodiments, pharmaceutical salt of Compound I is mono-salt. In certain embodiments, the pharmaceutical salt of Compound I is in amorphous form. In certain embodiments, the pharmaceutical salt of Compound I is in crystalline form. In certain embodiments, the pharmaceutical salt of Compound I is hydrochloric acid salt, L-(+)-tartaric acid salt, fumaric acid salt, sulfuric acid salt, and maleic acid salt of Compound I in crystalline form.

### Characterization of Crystalline Forms

In one aspect, the present disclosure provides several polymorphic crystalline forms of Compound I or pharmaceutically acceptable salts thereof.

### Crystalline Forms of Compound I or Salts Thereof

In one aspect, the present disclosure provides a crystalline form of Compound I, particularly, freebase Form A or Form B of Compound I. In another aspect, the present disclosure provides a crystalline form of a pharmaceutically acceptable salt of Compound I, particularly, crystalline form of hydrochloric acid salt of Compound I, crystalline form of L-(+)-tartaric acid salt of Compound I, crystalline form of fumaric acid salt of Compound I, crystalline form of sulfuric acid salt of Compound I, or crystalline form of maleic acid salt of Compound I.

### 1. Freebase Form A

In some embodiments, disclosed is crystalline form of Compound I (free base), which is Form A of Compound I.

In some embodiments, Form A of Compound I has an X-ray powder diffraction (XRPD) pattern comprising peaks at diffraction angles (2θ) of 11.62±0.20, 12.48±0.20, 17.34±0.20, and 20.04±0.20 degrees.

In some embodiments, Form A of Compound I has a XRPD pattern further comprising at least one, two, three or more peaks at 2θ selected from: 10.68±0.20, 11.11±0.20, 16.02±0.20, 20.79±0.20, 23.71±0.20, and 24.64±0.20 degrees.

In some embodiments, Form A of Compound I has a XRPD pattern comprising peaks at 2θ of 10.68±0.20, 11.11±0.20, 11.62±0.20, 12.48±0.20, 16.02±0.20, 17.34±0.20, 20.04±0.20, 20.79±0.20, 23.71±0.20, and 24.64±0.20 degrees.

In some embodiments, Form A of Compound I has a XRPD pattern further comprising at least one, two, three or more peaks at 2θ selected from: 5.95±0.20, 14.96±0.20, 22.01±0.20, and 27.60±0.20 degrees.

In some embodiments, Form A of Compound I has a XRPD pattern comprising peaks at 2θ of 5.95±0.20, 10.68±0.20, 11.11±0.20, 11.62±0.20, 12.48±0.20, 14.96±0.20, 16.02±0.20, 17.34±0.20, 20.04±0.20, 20.79±0.20, 22.01±0.20, 23.71±0.20, 24.64±0.20, and 27.60±0.20 degrees.

In some embodiments, Form A of Compound I has a XRPD pattern substantially as shown in Table 7.

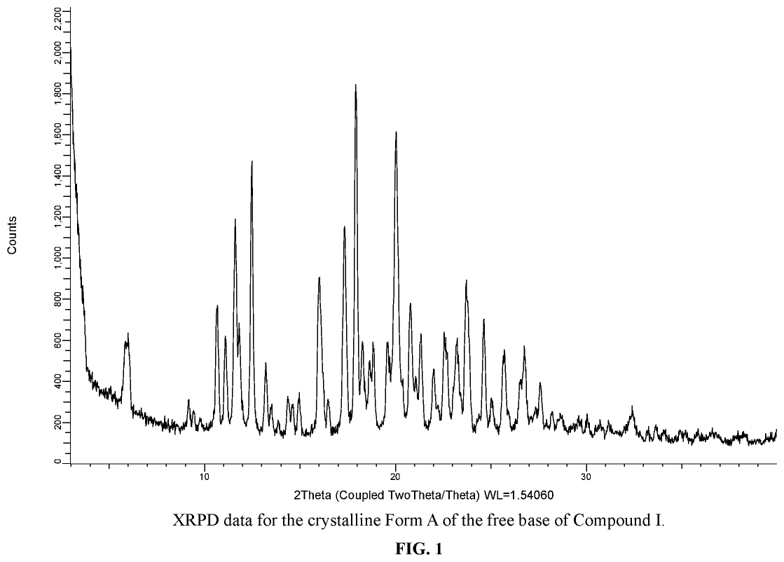

In some embodiments, Form A of Compound I has a XRPD pattern substantially as shown in FIG. 1.

In some embodiments, Form A of Compound I has a DSC thermogram comprising an endotherm with a desolvation onset at about 178.6° C. and a peak at about 179.6° C.

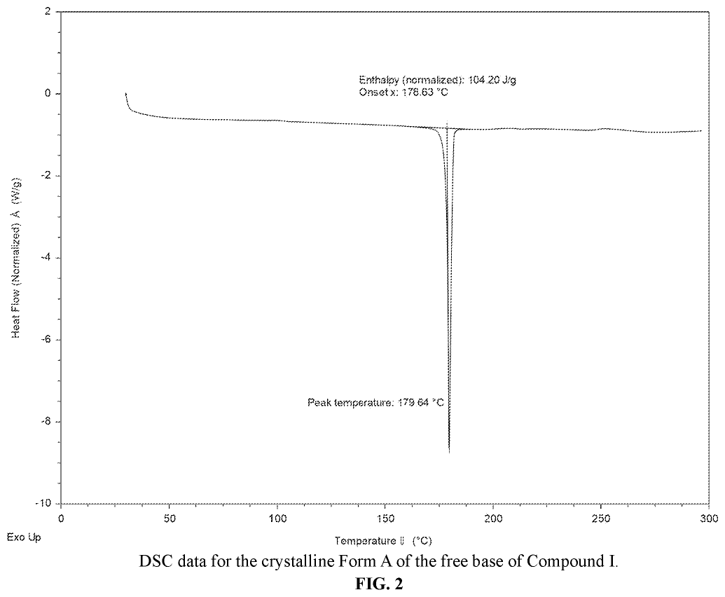

In some embodiments, Form A of Compound I has a DSC thermogram substantially similar to FIG. 2.

In some embodiments, Form A of Compound I has a TGA thermogram exhibiting a mass loss of about 0.23% upon heating from about 38° C. to about 160° C.

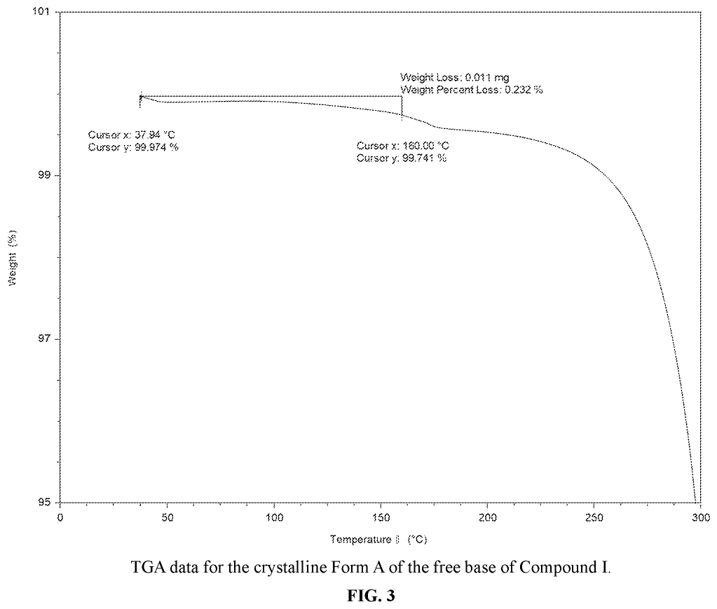

In some embodiments, Form A of Compound I has a TGA thermogram substantially similar to FIG. 3

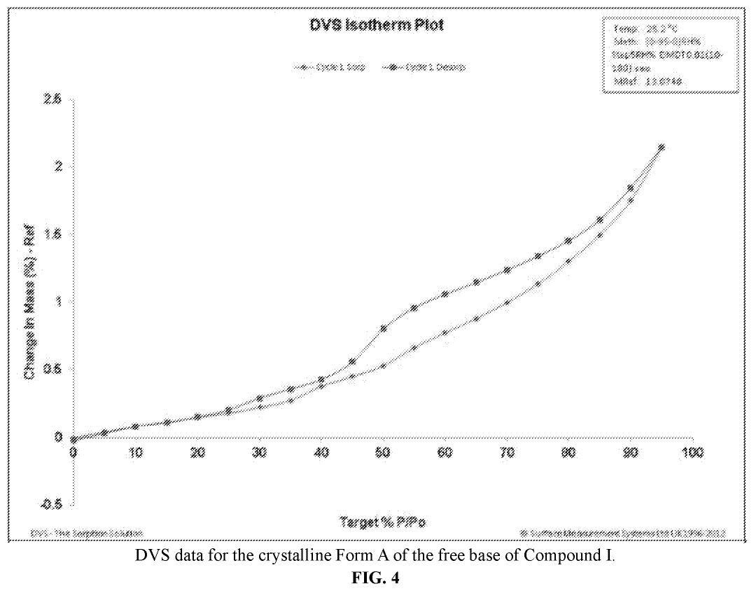

In some embodiments, Form A of Compound I has a DVS vapor sorption gram substantially similar to FIG. 4.

### 2. Freebase Form B

In some embodiments, disclosed is crystalline form of Compound I (free base), which is Form B of Compound I.

In some embodiments, Form B of Compound I has a XRPD pattern comprising peaks at 2θ of 9.39±0.20, 18.86±0.20, 19.50±0.20, and 20.06±0.20 degrees.

In some embodiments, Form B of Compound I has a XRPD pattern further comprising at least one, two, three or more peaks at 2θ selected from: 10.59±0.20, 18.16±0.20, 18.56±0.20, 26.30±0.20, 33.71±0.20, and 34.81±0.20 degrees.

In some embodiments, Form B of Compound I has a XRPD pattern comprising peaks at 2θ of 9.39±0.20, 10.59±0.20, 18.16±0.20, 18.56±0.20, 18.86±0.20, 19.50±0.20, 20.06±0.20, 26.30±0.20, 33.71±0.20, and 34.81±0.20 degrees.

In some embodiments, Form B of Compound I has a XRPD pattern further comprising at least one, two, three or more peaks at 2θ selected from: 22.07±0.20, 22.91±0.20, 23.68±0.20, and 24.00±0.20 degrees.

In some embodiments, Form B of Compound I has a XRPD pattern comprising peaks at 2θ of 9.39±0.20, 10.59±0.20, 18.16±0.20, 18.56±0.20, 18.86±0.20, 19.50±0.20, 20.06±0.20, 22.07±0.20, 22.91±0.20, 23.68±0.20, 24.00±0.20, 26.30±0.20, 33.71±0.20, and 34.81±0.20 degrees.

In some embodiments, Form B of Compound I has a XRPD pattern substantially as shown in Table 8.

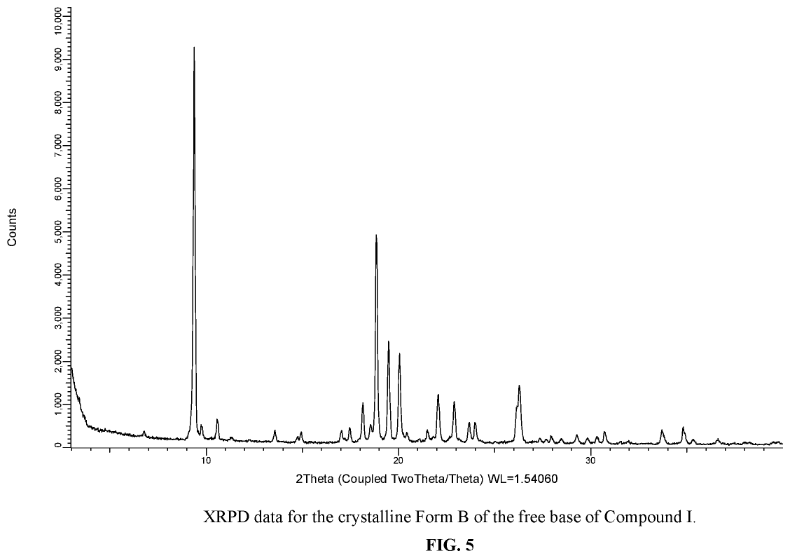

In some embodiments, Form B of Compound I has a XRPD pattern substantially as shown in FIG. 5.

In some embodiments, Form B of Compound I has a DSC thermogram comprising an endotherm with a desolvation onset at about 194.8° C. and a peak at about 196.7° C.

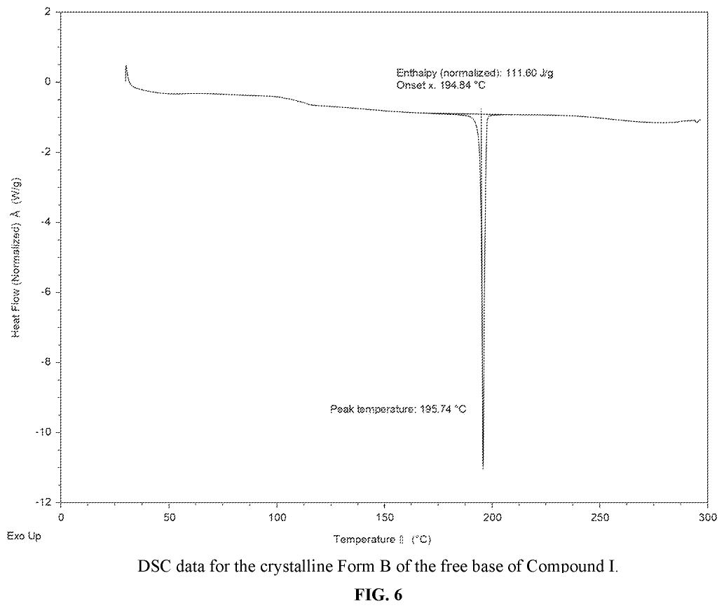

In some embodiments, Form B of Compound I has a DSC thermogram substantially similar to FIG. 6.

In some embodiments, Form B of Compound I has a TGA thermogram exhibiting a mass loss of less than 0.17% upon heating from about 38° C. to about 178° C.

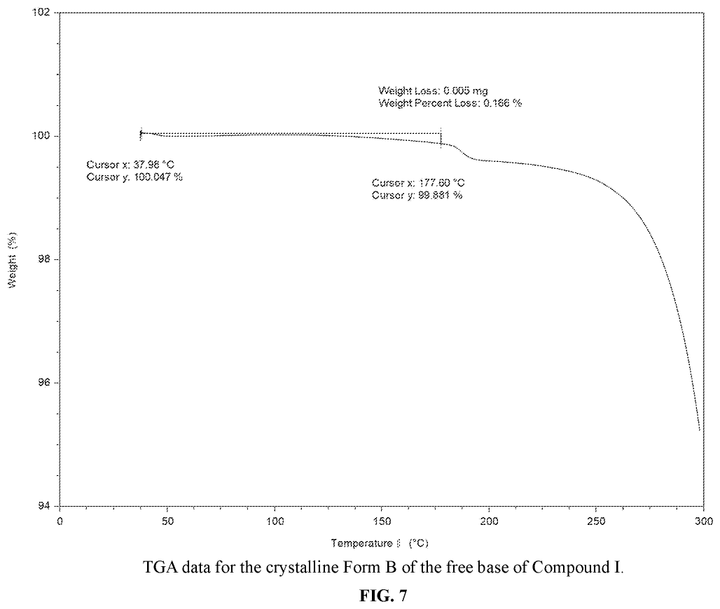

In some embodiments, Form B of Compound I has a TGA thermogram substantially similar to FIG. 7.

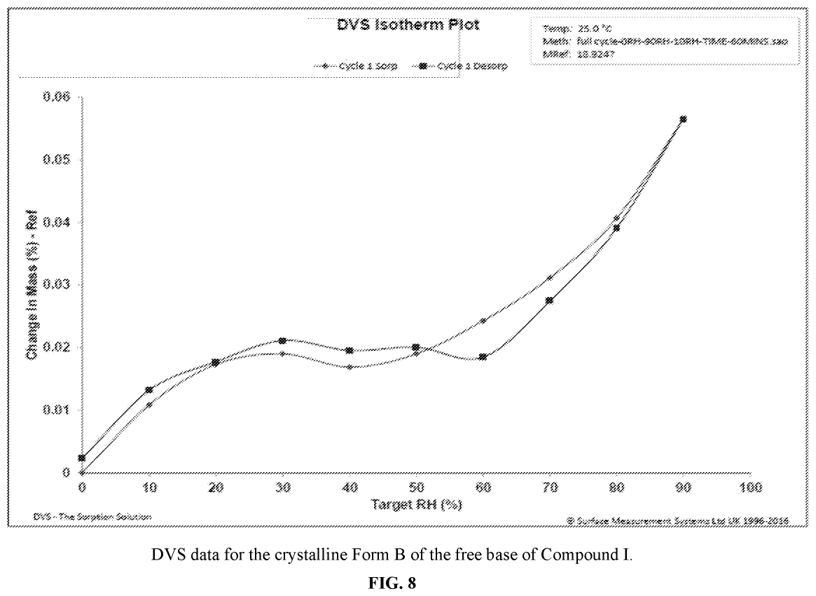

In some embodiments, Form B of Compound I has a DVS vapor sorption gram substantially similar to FIG. 8.

### 3. Crystalline Form of Compound I Hydrochloric Acid Salt

In some embodiments, disclosed is a crystalline form of a pharmaceutically acceptable salt of Compound I, which is a crystalline form of Compound I hydrochloric acid salt.

In some embodiments, the crystalline form of Compound I hydrochloric acid salt has a XRPD pattern comprising peaks at 2θ of 9.35±0.20, 17.21±0.20, 18.21±0.20, 19.79±0.20, and 21.17±0.20 degrees.

In some embodiments, the crystalline form of Compound I hydrochloric acid salt has a XRPD pattern further comprising at least one, two, three or more peaks at 20 selected from: 9.05±0.20, 19.54±0.20, 21.17±0.20, 21.51±0.20, 26.24±0.20, and 30.64±0.20 degrees.

In some embodiments, the crystalline form of Compound I hydrochloric acid salt has a XRPD pattern comprising peaks at 2θ of 9.05±0.20, 9.35±0.20, 17.21±0.20, 18.21±0.20, 19.54±0.20, 19.79±0.20, 21.17±0.20, 21.51±0.20, 26.24±0.20, and 30.64±0.20 degrees.

In some embodiments, the crystalline form of Compound I hydrochloric acid salt has a XRPD pattern further comprising at least one, two, three or more peaks at 2θ selected from: 7.30±0.20, 14.85±0.20, 20.91±0.20, 23.25±0.20, and 27.43±0.20 degrees.

In some embodiments, the crystalline form of Compound I hydrochloric acid salt has a XRPD pattern comprising peaks at 2θ of 7.30±0.20, 9.05±0.20, 9.35±0.20, 14.85±0.20, 17.21±0.20, 18.21±0.20, 19.54±0.20 19.79±0.20, 20.91±0.20, 21.17±0.20, 21.51±0.20, 23.25±0.20, 26.24±0.20, 27.43±0.20, and 30.64±0.20 degrees.

In some embodiments, the crystalline form of Compound I hydrochloric acid salt has a XRPD pattern substantially as shown in Table 16.

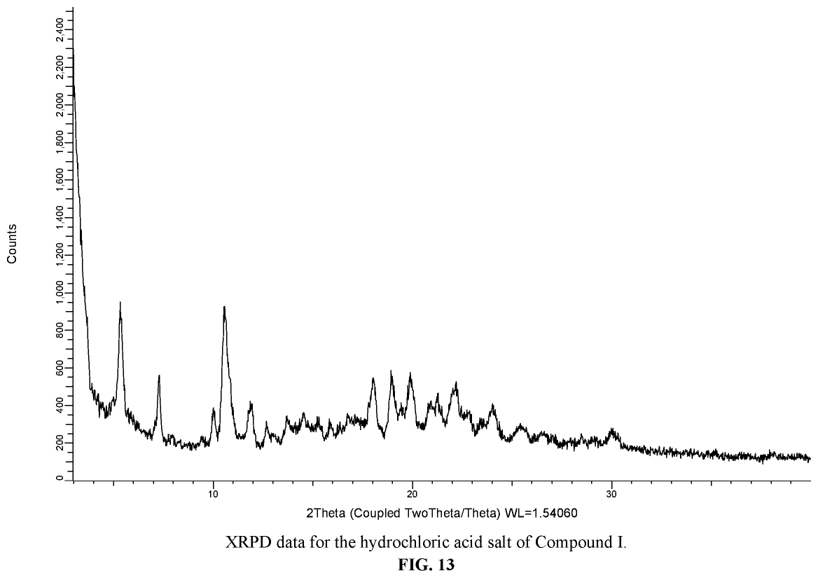

In some embodiments, the crystalline form of Compound I hydrochloric acid salt has a XRPD pattern substantially as shown in FIG. 13.

In some embodiments, the crystalline form of Compound I hydrochloric acid salt has a DSC thermogram comprising an endotherm with a desolvation onset at about 207.8° C. and a peak at about 212.1° C.

In some embodiments, the crystalline form of Compound I hydrochloric acid salt has a TGA thermogram exhibiting a mass loss of about 0.76% upon heating to about 175° C.

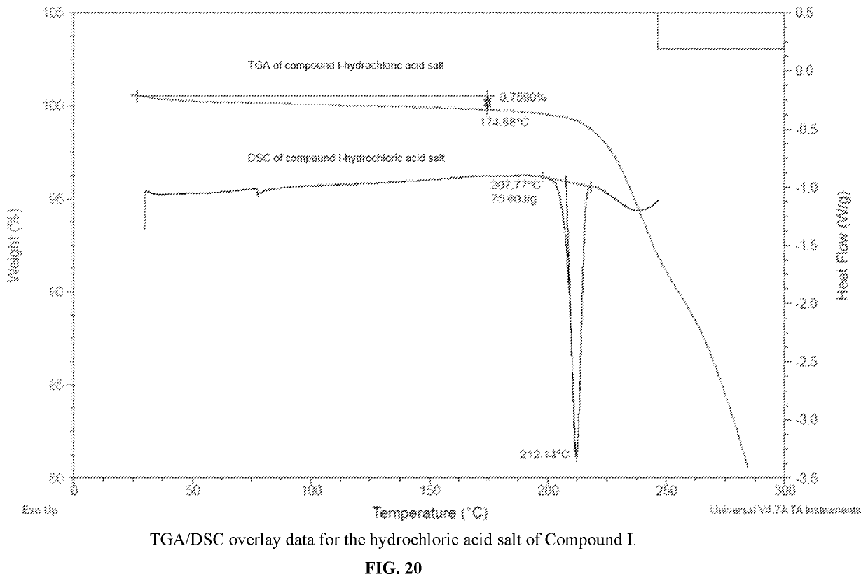

In some embodiments, the crystalline form of Compound I hydrochloric acid salt has a TGA/DSC thermogram substantially similar to FIG. 20.

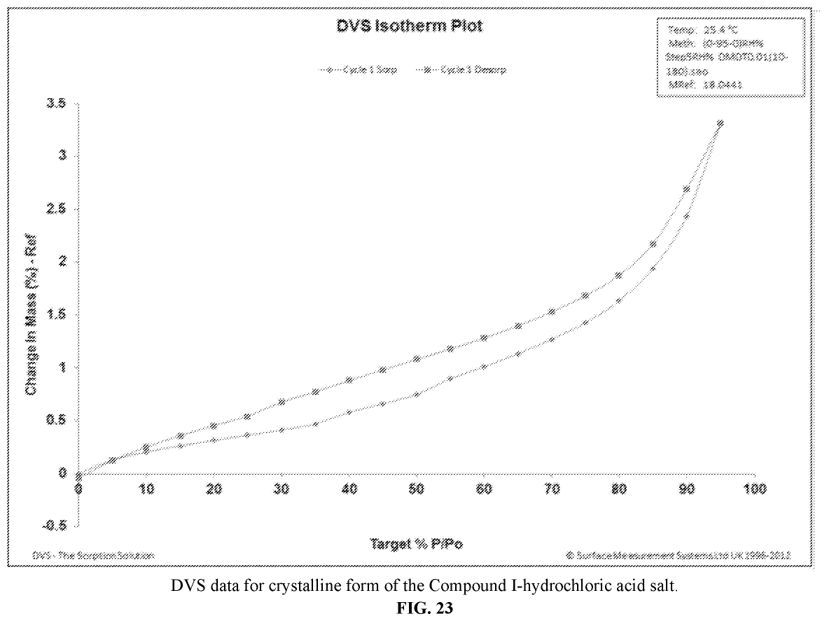

In some embodiments, the crystalline form of Compound I hydrochloric acid salt has a DVS vapor sorption gram substantially similar to FIG. 23.

4. Crystalline Form of Compound I L-(+)-tartaric acid salt Pattern I

In some embodiments, disclosed is a crystalline form of a pharmaceutically acceptable salt of Compound I, which is a crystalline form of Compound I L-(+)-tartaric acid salt Pattern I.

In some embodiments, the crystalline form of Compound I L-(+)-tartaric acid salt pattern I has a XRPD pattern comprising peaks at 2θ of 5.34±0.20, 5.38±0.20, 10.50±0.20, 10.92±0.20, and 16.37±0.20 degrees.

In some embodiments, the crystalline form of Compound I L-(+)-tartaric acid salt pattern I has a XRPD pattern further comprising at least one, two, three or more peaks at 2θ selected from: 11.84±0.20, 15.05±0.20, 17.86±0.20, 18.52±0.20, and 18.99±0.20 degrees.

In some embodiments, the crystalline form of Compound I L-(+)-tartaric acid salt pattern I has a XRPD pattern comprising peaks at 2θ of 5.34±0.20, 5.38±0.20, 10.50±0.20, 10.92±0.20, 11.84±0.20, 15.05±0.20, 16.37±0.20, 17.86±0.20, 18.52±0.20, and 18.99±0.20 degrees.

In some embodiments, the crystalline form of Compound I L-(+)-tartaric acid salt pattern I has a XRPD pattern further comprising at least one, two, three or more peaks at 2θ selected from: 7.29±0.20, 14.40±0.20, 22.02±0.20, and 23.96±0.20 degrees.

In some embodiments, the crystalline form of Compound I L-(+)-tartaric acid salt pattern I has a XRPD pattern comprising peaks at 2θ of 5.34±0.20, 5.38±0.20, 7.29±0.20, 10.50±0.20, 10.92±0.20, 11.84±0.20, 14.40±0.20, 15.05±0.20, 16.37±0.20, 17.86±0.20, 18.52±0.20, 18.99±0.20 22.02±0.20, and 23.96±0.20 degrees.

In some embodiments, the crystalline form of Compound I L-(+)-tartaric acid salt pattern I has a XRPD pattern substantially as shown in Table 17.

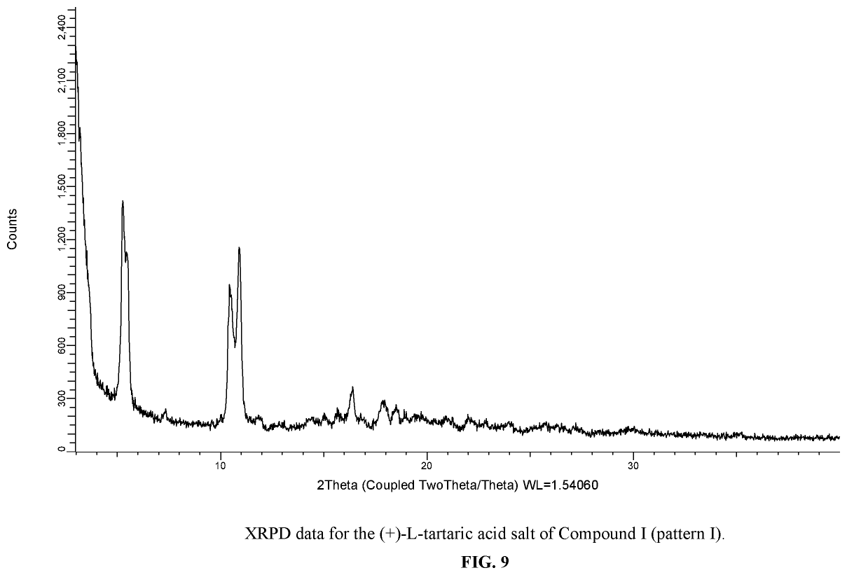

In some embodiments, the crystalline form of Compound I L-(+)-tartaric acid salt pattern I has a XRPD pattern substantially as shown in FIG. 9.

In some embodiments, the crystalline form of Compound I L-(+)-tartaric acid salt pattern I has a DSC thermogram comprising an endotherm with a desolvation onset at about 207.8° C. and a peak at about 212.1° C.

In some embodiments, the crystalline form of Compound I L-(+)-tartaric acid salt pattern I has a TGA thermogram exhibiting a mass loss of about 0.76% upon heating to about 175° C.

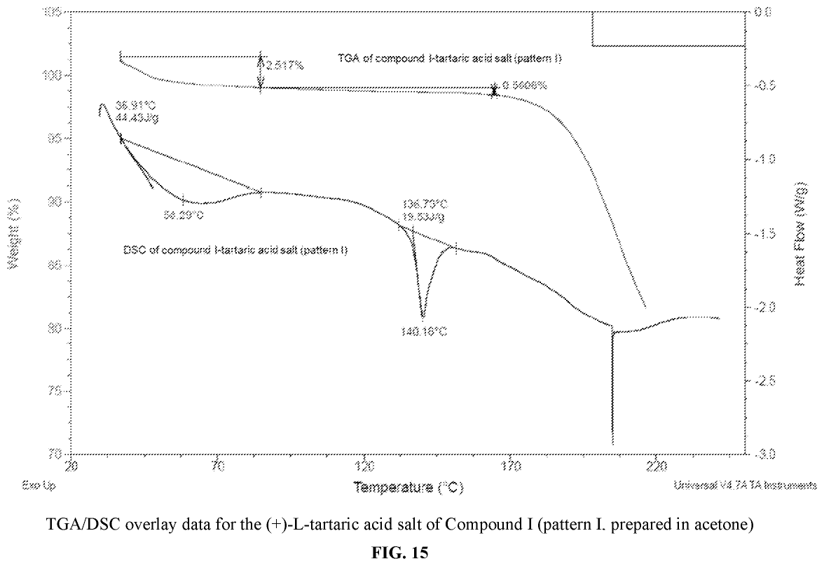

In some embodiments, the crystalline form of Compound I L-(+)-tartaric acid salt pattern I has a TGA thermogram substantially similar to FIG. 15.

5. Crystalline Form of Compound I L-(+)-tartaric acid salt Pattern II

In some embodiments, disclosed is a crystalline form of a pharmaceutically acceptable salt of Compound I, which is a crystalline form of Compound I L-(+)-tartaric acid salt Pattern II.

In some embodiments, the crystalline form of Compound I L-(+)-tartaric acid salt pattern II has a XRPD pattern comprising peaks at 2θ of 10.02±0.20, 18.03±0.20, 19.89±0.20, 21.15±0.20, and 21.26±0.20 degrees.

In some embodiments, the crystalline form of Compound I L-(+)-tartaric acid salt pattern II has a XRPD pattern further comprising at least one, two, three or more peaks at 2θ selected from: 12.70±0.20, 13.76±0.20, 16.80±0.20, 20.92±0.20, and 22.82±0.20 degrees.

In some embodiments, the crystalline form of Compound I L-(+)-tartaric acid salt pattern II has a XRPD pattern comprising peaks at 2θ of 10.02±0.20, 12.70±0.20, 13.76±0.20, 16.80±0.20, 18.03±0.20, 19.89±0.20, 20.92±0.20, 21.15±0.20, 21.26±0.20, and 22.82±0.20 degrees.

In some embodiments, the crystalline form of Compound I L-(+)-tartaric acid salt pattern II has a XRPD pattern further comprising at least one, two, three or more peaks at 2θ selected from: 7.95±0.20, 15.91±0.20, 23.44±0.20, 25.55±0.20, and 29.99±0.20 degrees.

In some embodiments, the crystalline form of Compound I L-(+)-tartaric acid salt pattern II has a XRPD pattern comprising peaks at 2θ of 7.95±0.20, 10.02±0.20, 12.70±0.20, 13.76±0.20, 15.91±0.20, 16.80±0.20, 18.03±0.20, 19.89±0.20, 20.92±0.20, 21.15±0.20, 21.26±0.20, 22.82±0.20, 23.44±0.20, 25.55±0.20, and 29.99±0.20 degrees.

In some embodiments, the crystalline form of Compound I L-(+)-tartaric acid salt pattern II has a XRPD pattern substantially as shown in Table 21.

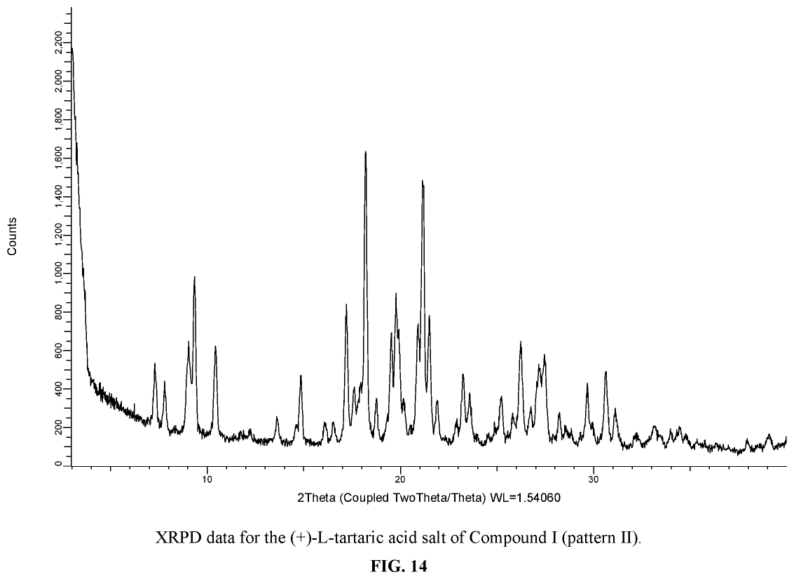

In some embodiments, the crystalline form of Compound I L-(+)-tartaric acid salt pattern II has a XRPD pattern substantially as shown in FIG. 14.

In some embodiments, the crystalline form of Compound I L-(+)-tartaric acid salt pattern II has a DSC thermogram comprising an endotherm with a desolvation onset at about 137.2° C. and a peak at about 140.4° C.

In some embodiments, the crystalline form of Compound I L-(+)-tartaric acid salt pattern II has a TGA thermogram exhibiting a mass loss of about 3.59% upon heating to about 100° C.

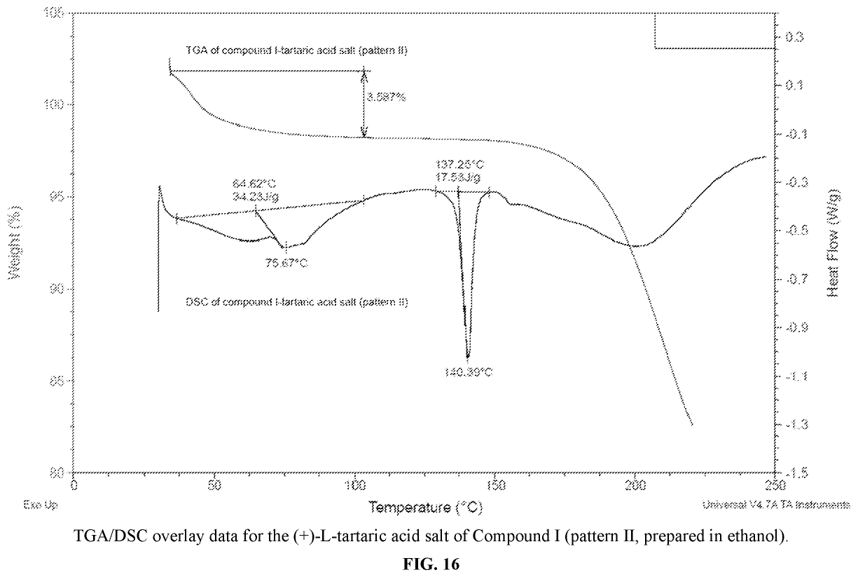

In some embodiments, the crystalline form of Compound I L-(+)-tartaric acid salt pattern II has a TGA/DSC thermogram substantially similar to FIG. 16.

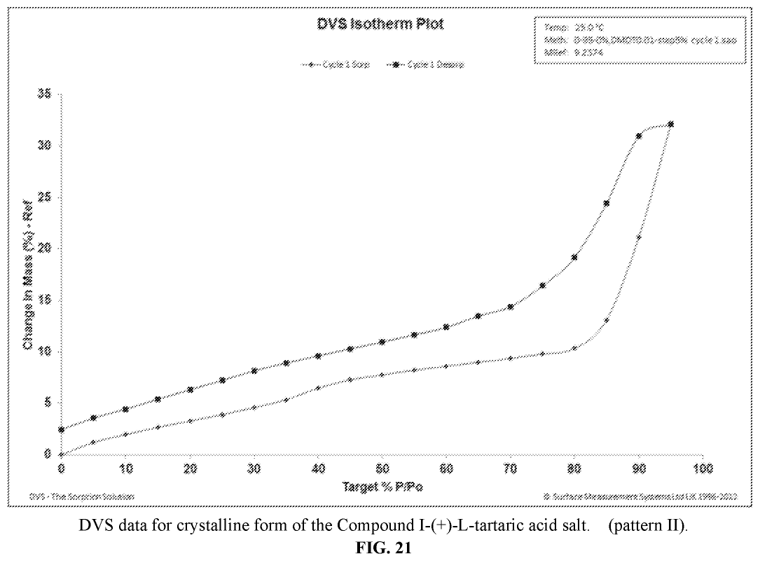

In some embodiments, the crystalline form of Compound I L-(+)-tartaric acid salt pattern II has a DVS vapor sorption gram substantially similar to FIG. 21.

### 6. Crystalline Form of Compound I Fumaric Acid Salt

In some embodiments, disclosed is a crystalline form of a pharmaceutically acceptable salt of Compound I, which is a crystalline form of Compound I fumaric acid salt.

In some embodiments, the crystalline form of Compound I fumaric acid salt has a XRPD pattern comprising peaks at 2θ of 11.92±0.20, 13.71±0.20, 19.54±0.20, 20.15±0.20, and 24.21±0.20 degrees.

In some embodiments, the crystalline form of Compound I fumaric acid salt has a XRPD pattern further comprising at least one, two, three or more peaks at 2θ selected from: 13.08±0.20, 15.79±0.20, 18.86±0.20, 20.63±0.20, and 22.14±0.20 degrees.

In some embodiments, the crystalline form of Compound I fumaric acid salt has a XRPD pattern comprising peaks at 2θ of 11.92±0.20, 13.08±0.20, 13.71±0.20, 15.79±0.20, 19.54±0.20, 20.15±0.20, 18.86±0.20, 20.63±0.20, 22.14±0.20, and 24.21±0.20 degrees.

In some embodiments, the crystalline form of Compound I fumaric acid salt has a XRPD pattern further comprising at least one, two, three or more peaks at 2θ selected from: 11.63±0.20, 12.33±0.20, 17.23±0.20, 18.52±0.20, and 23.79±0.20 degrees.

In some embodiments, the crystalline form of Compound I fumaric acid salt has a XRPD pattern comprising peaks at 2θ of 11.63±0.20, 11.92±0.20, 12.33±0.20, 13.08±0.20, 13.71±0.20, 15.79±0.20, 17.23±0.20, 18.52±0.20, 18.86±0.20, 19.54±0.20, 20.15±0.20, 20.63±0.20, 22.14±0.20, 23.79±0.20, and 24.21±0.20 degrees.

In some embodiments, the crystalline form of Compound I fumaric acid salt has a XRPD pattern substantially as shown in Table 18.

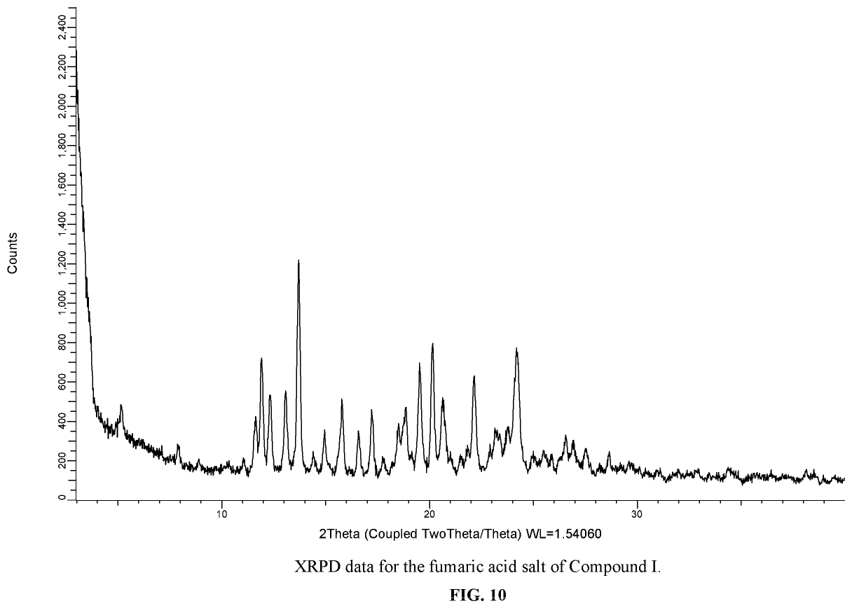

In some embodiments, the crystalline form of Compound I fumaric acid salt has a XRPD pattern substantially as shown in FIG. 10.

In some embodiments, the crystalline form of Compound I fumaric acid salt has a DSC thermogram comprising an endotherm with a desolvation onset at about 48.9° C. and a peak at about 68.3° C.

In some embodiments, the crystalline form of Compound I fumaric acid salt has a DSC thermogram further comprising an later endotherm with a desolvation onset at about 132.79° C. and a peak at about 141.78° C.

In some embodiments, the crystalline form of Compound I fumaric acid salt has a TGA thermogram exhibiting a mass loss of about 2.86% upon heating to about 55° C.

In some embodiments, the crystalline form of Compound I fumaric acid salt has a TGA thermogram exhibiting a mass loss of about 2.42% upon heating from about 55° C. to about 140° C.

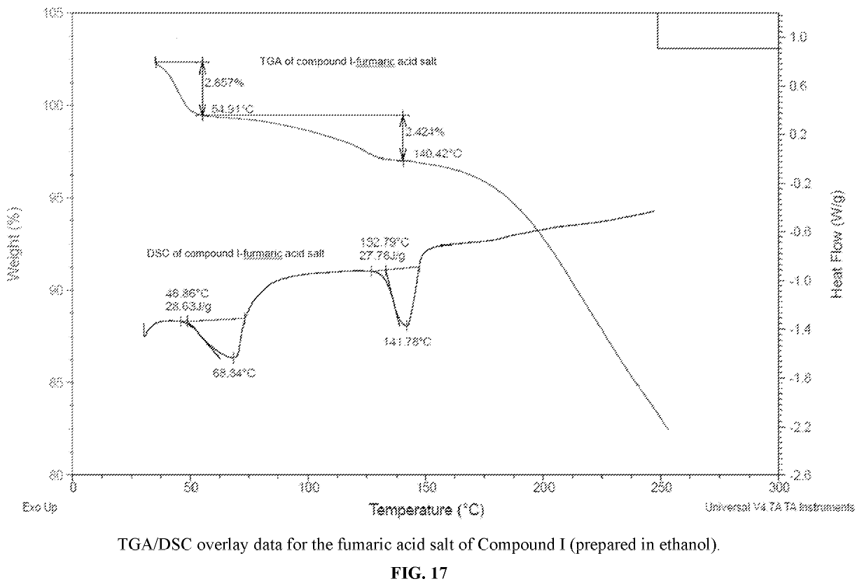

In some embodiments, the crystalline form of Compound I fumaric acid salt has a TGA/DSC thermogram substantially similar to FIG. 17.

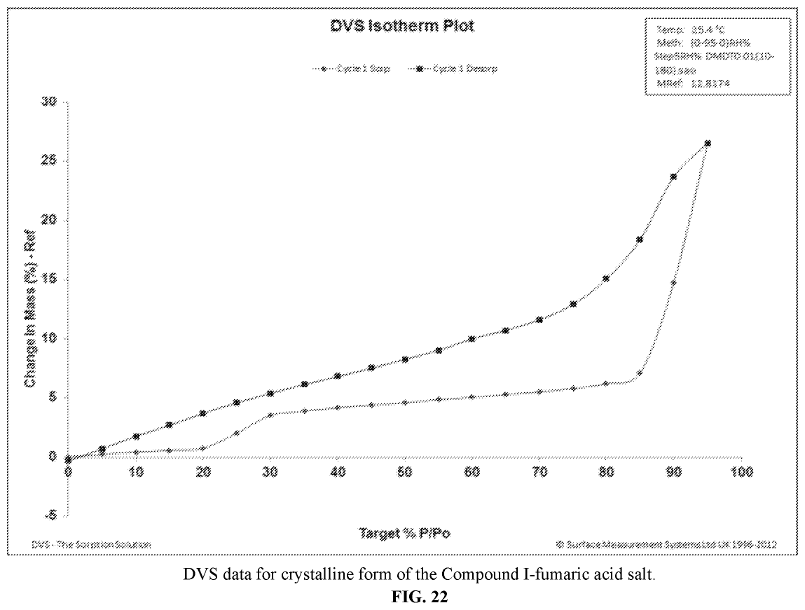

In some embodiments, the crystalline form of Compound I fumaric acid salt has a DVS vapor sorption gram substantially similar to FIG. 22.

### 7. Crystalline Form of Compound I Sulfuric Acid Salt

In some embodiments, disclosed is a crystalline form of a pharmaceutically acceptable salt of Compound I, which is a crystalline form of Compound I sulfuric acid salt.

In some embodiments, the crystalline form of Compound I sulfuric acid salt has a XRPD pattern comprising peaks at 2θ of 6.00±0.20, 12.16±0.20, 17.37±0.20, 18.19±0.20, and 20.51±0.20 degrees.

In some embodiments, the crystalline form of Compound I sulfuric acid salt has a XRPD pattern further comprising at least one, two, three or more peaks at 2θ selected from: 7.54±0.20, 17.16±0.20, 19.52±0.20, and 22.65±0.20 degrees.

In some embodiments, the crystalline form of Compound I sulfuric acid salt has a XRPD pattern comprising peaks at 2θ of 6.00±0.20, 7.54±0.20, 12.16±0.20, 17.16±0.20, 17.37±0.20, 18.19±0.20, 19.52±0.20, 20.51±0.20, and 22.65±0.20 degrees.

In some embodiments, the crystalline form of Compound I sulfuric acid salt has a XRPD pattern further comprising at least one, two, three or more peaks at 2θ selected from: 14.90±0.20, 22.02±0.20, 24.86±0.20, and 25.73±0.20 degrees.

In some embodiments, the crystalline form of Compound I sulfuric acid salt has a XRPD pattern comprising peaks at 2θ of 6.00±0.20, 7.54±0.20, 12.16±0.20, 14.90±0.20, 17.16±0.20, 17.37±0.20, 18.19±0.20, 19.52±0.20, 20.51±0.20, 22.02±0.20, 22.65±0.20, 24.86±0.20, and 25.73±0.20 degrees.

In some embodiments, the crystalline form of Compound I sulfuric acid salt has a XRPD pattern substantially as shown in Table 19.

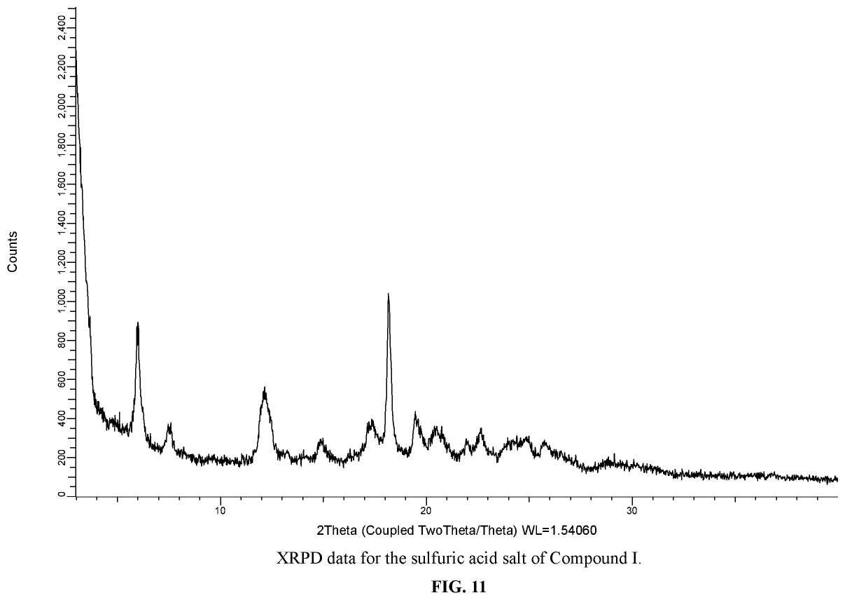

In some embodiments, the crystalline form of Compound I sulfuric acid salt has a XRPD pattern substantially as shown in FIG. 11.

In some embodiments, the crystalline form of Compound I sulfuric acid salt has a DSC thermogram comprising an endotherm with a desolvation onset at about 181.2° C. and a peak at about 195.9° C.

In some embodiments, the crystalline form of Compound I sulfuric acid salt has a DSC thermogram further comprising an endotherm with a later desolvation onset at about 210.6° C. and a peak at about 226.0° C.

In some embodiments, the crystalline form of Compound I sulfuric acid salt has a TGA thermogram exhibiting a mass loss of about 4.85% upon heating to about 120° C.

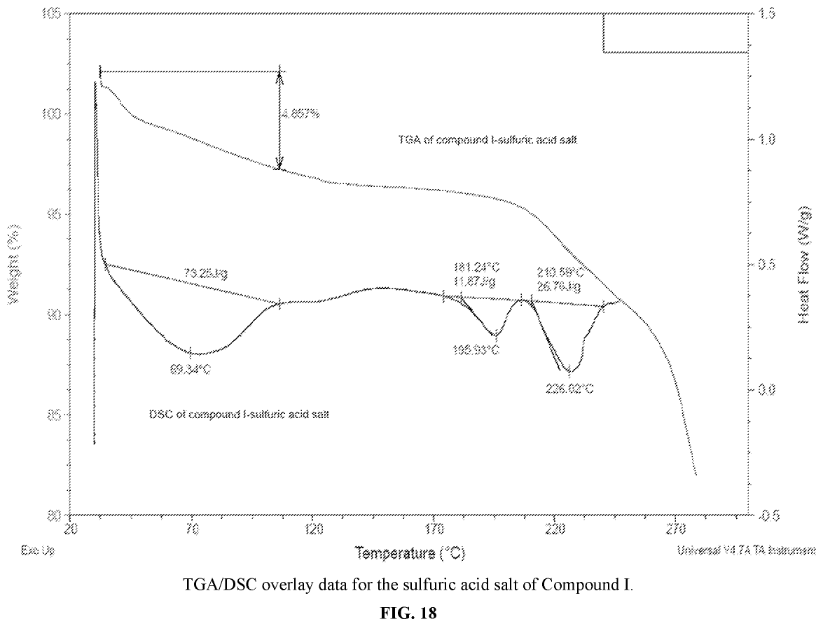

In some embodiments, the crystalline form of Compound I sulfuric acid salt has a TGA thermogram substantially similar to FIG. 18.

### 8. Crystalline Form of Compound I Maleic Acid Salt

In some embodiments, disclosed is a crystalline form of a pharmaceutically acceptable salt of Compound I, which is a crystalline form of Compound I maleic acid salt.

In some embodiments, the crystalline form of Compound I maleic acid salt has a XRPD pattern comprising peaks at 2θ of 11.94±0.20, 15.64±0.20, 16.10±0.20, 20.98±0.20, and 22.65±0.20 degrees.

In some embodiments, the crystalline form of Compound I maleic acid salt has a XRPD pattern further comprising at least one, two, three or more peaks at 2θ selected from: 4.90±0.20, 7.45±0.20, 24.27±0.20, and 25.67±0.20 degrees.

In some embodiments, the crystalline form of Compound I maleic acid salt has a XRPD pattern comprising peaks at 2θ of 4.90±0.20, 7.45±0.20, 11.94±0.20, 15.64±0.20, 16.10±0.20, 20.98±0.20, 22.65±0.20, 24.27±0.20, and 25.67±0.20 degrees.

In some embodiments, the crystalline form of Compound I maleic acid salt has a XRPD pattern further comprising at least one, two, three or more peaks at 2θ selected from: 9.57±0.20, 12.74±0.20, 13.19±0.20, and 18.46±0.20 degrees.

In some embodiments, the crystalline form of Compound I maleic acid salt has a XRPD pattern comprising peaks at 2θ of 4.90±0.20, 7.45±0.20, 9.57±0.20, 11.94±0.20, 12.74±0.20, 13.19±0.20, 15.64±0.20, 16.10±0.20, 18.46±0.20, 20.98±0.20, 22.65±0.20, 24.27±0.20, and 25.67±0.20 degrees.

In some embodiments, the crystalline form of Compound I maleic acid salt has a XRPD pattern substantially as shown in Table 20.

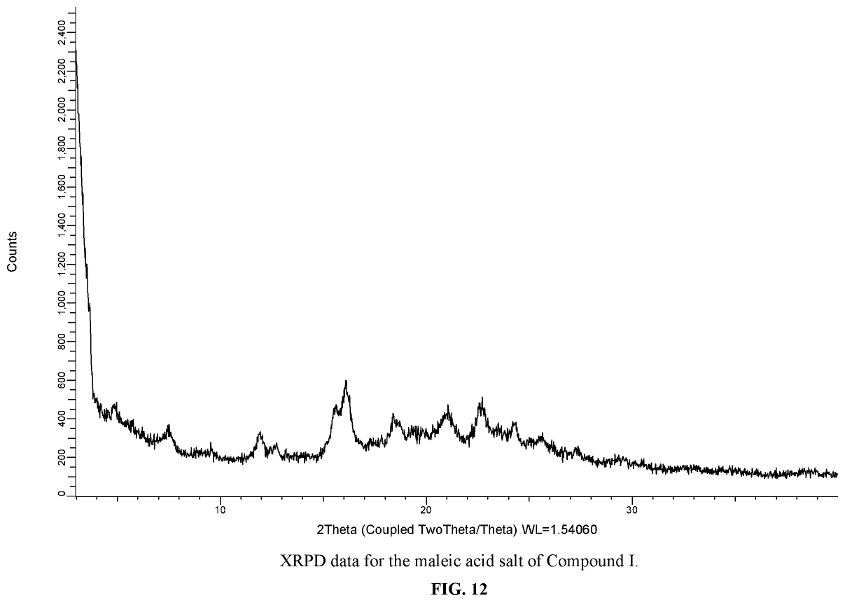

In some embodiments, the crystalline form of Compound I maleic acid salt has a XRPD pattern substantially as shown in FIG. 12.

In some embodiments, the crystalline form of Compound I maleic acid salt has a DSC thermogram comprising an endotherm with a desolvation onset at about 64.6° C. and a peak at about 75.7° C.

In some embodiments, the crystalline form of Compound I maleic acid salt has a DSC thermogram further comprising an endotherm with a later desolvation onset at about 137.3° C. and a peak at about 140.4° C.

In some embodiments, the crystalline form of Compound I maleic acid salt has a TGA thermogram exhibiting a mass loss of about 3.59% upon heating to about 100° C.

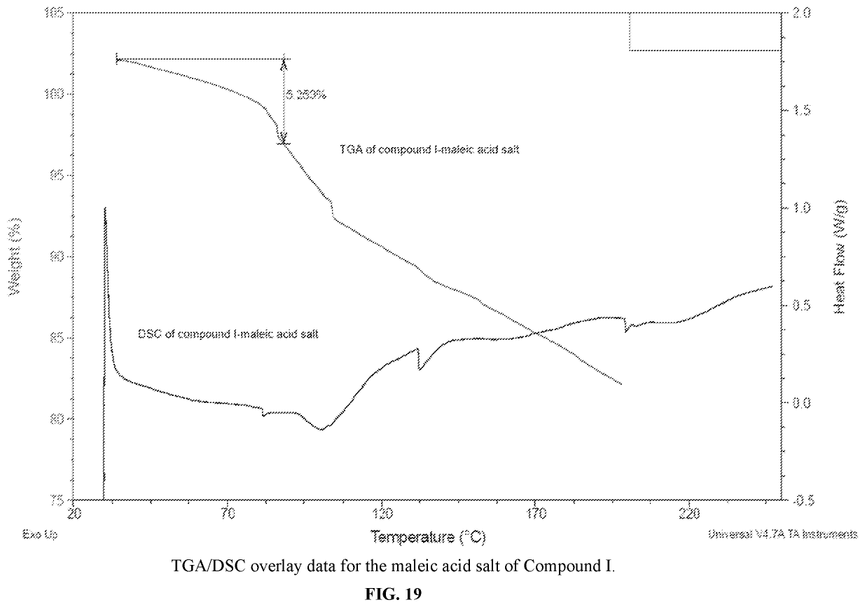

In some embodiments, the crystalline form of Compound I maleic acid salt has a TGA thermogram substantially similar to FIG. 19.

When a crystalline form is referred herein, the degree of crystallinity is conveniently greater than about 60%, more conveniently greater than about 80%, conveniently greater than about 90% and more conveniently greater than about 95%. Most conveniently the degree of crystallinity is greater than about 98%.

In some embodiments, the polymorphic forms of the present disclosure are preferably substantially pure, meaning each polymorph form includes no more than 10%, preferably no more than 5%, and preferably no more than 1% by weight of any one apparent impurity, including other polymorphic forms of the compound. In certain embodiments, a “substantially pure” polymorphic form of the present disclosure has a purity of over 90%, over 95%, over 98% or even over 99%.

In some embodiments, the polymorphic forms of the present disclosure may also exist together in a mixture. Mixtures of polymorphic forms of the present disclosure will have XRPD peaks characteristic of each of the polymorphic forms present in the mixture. For example, a mixture of two polymorphs will have a XRPD pattern that is a convolution of the X-ray powder diffraction patterns corresponding to the substantially pure polymorphs.

### Processes for Preparation

Further provided herein are the processes for the preparation of pharmaceutically acceptable salts and polymorphic forms of Compound I and the pharmaceutically acceptable salts thereof.

The pharmaceutical salts and polymorphic forms of the present disclosure may be prepared by the methods known in the art. In some embodiments, the crystals of pharmaceutically acceptable salt of Compound I are prepared by dissolving Compound I in acetone or ethanol solution, adding corresponding acid in acetone or ethanol solution, and leaving the solution to crystallize and isolating the crystals of the pharmaceutically acceptable salt of Compound I, wherein the pharmaceutically acceptable salt is selected from hydrochloric acid salt, L-(+)-tartaric acid salt, fumaric acid salt, sulfuric acid salt, and maleic acid salt. However, these are by no means limiting the preparation methods of the pharmaceutical salts and polymorphic forms of the present disclosure.

Further provided herein is a process for preparing Compound I on tens of kilogram scale with high product yield.

The process for preparing Compound I on tens of kilogram scale is summarized in the scheme below:

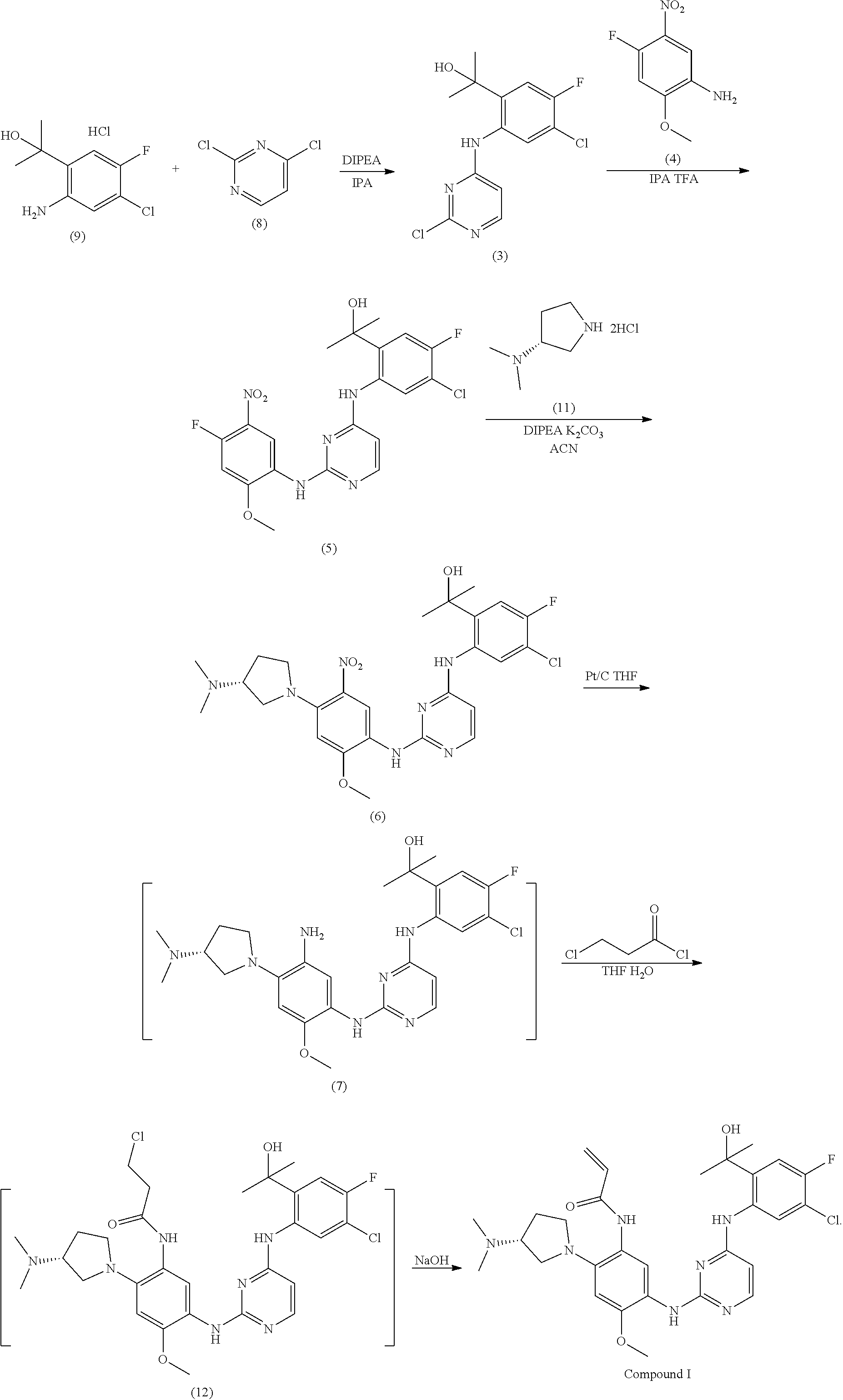

The improved process summarized in the scheme above was shown to be suitable for manufacture of Compound I on tens of kilogram scale with high yield. In particular:
(i) It is not necessary to isolate the compound of Formula (7) in the process;
(ii) The compound of Formula (9) is selected and used for producing the compound of Formula (3), which significantly increases the yield of the compound of Formula (3); In some embodiments, the yield of the compound of Formula (3) is increased by 29% compared to the method disclosed in WO2019149164A1; and
(iii) The process adopts the particular synthetic route from the compound having the structure of Formula (6) to Compound I, which significantly increases product yield of Compound I. In some embodiments, the yield of Compound I is increased by 74% compared to the method disclosed in WO2019149164A1.

In some embodiments, the process for preparing Compound I comprises a step of (i) contacting a compound of Formula (7):

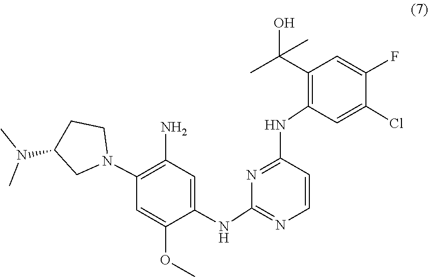 [SMILES: COc1cc(N2CC[C@@H](N(C)C)C2)c(N)cc1Nc1nccc(Nc2cc(Cl)c(F)cc2C(C)(C)O)n1]

with an acrylamide reagent, and (ii) adding a base reagent into the mixture obtained in the step (i) to form Compound I. In some embodiments, the acrylamide reagent is selected from the group consisting of: acryloyl chloride, acrylic acid, 3-chloropropionic acid and the 3-chloropropionyl chloride. In some embodiments, the acrylamide reagent is 3-chloropropionyl chloride. In some embodiments, the base reagent is selected from the group consisting of N,N-diisopropylethylamine, Triethylamine, pyridine, DBU, K₂CO₃, KOH, KHCO₃, LiOH, NaOH, Na₂CO₃, NaHCO₃. In some embodiments, the base reagent is NaOH.

In some embodiments, the process for preparing Compound I comprises further step of (iii) preparing the compound of Formula (7) by contacting a compound of Formula (6):

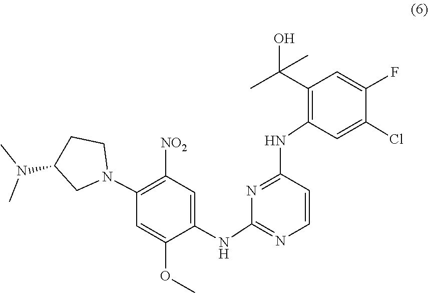 [SMILES: COc1cc(N2CC[C@@H](N(C)C)C2)c([N+](=O)[O-])cc1Nc1nccc(Nc2cc(Cl)c(F)cc2C(C)(C)O)n1]

with an organic solvent in the presence of a palladium catalyst. In some embodiments, the organic solvent is Tetrahydrofuran. In some embodiments, the compound of Formula (7) obtained in step (iii) is not isolated and is directly used in the step of (i).

In some embodiments, the process for preparing Compound I comprises further step of (iv) preparing the compound of Formula (6) by contacting a compound of Formula (5):

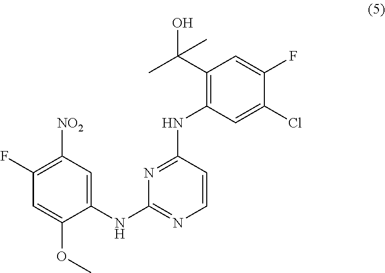 [SMILES: COc1cc(F)c([N+](=O)[O-])cc1Nc1nccc(Nc2cc(Cl)c(F)cc2C(C)(C)O)n1]

with a compound of Formula (10) or Formula (11):

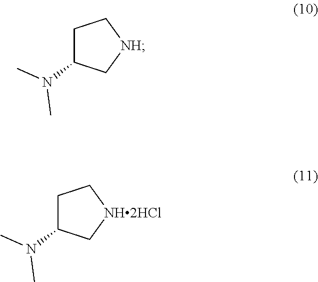

in the presence of a base and an organic solvent. In some embodiments, the base is K₂CO₃ and/or N,N-Diisopropylethylamine and the organic solvent is acetonitrile.

In some embodiments, the process for preparing Compound I comprises further step of (v) preparing the compound of Formula (5) by contacting a compound of Formula (3):

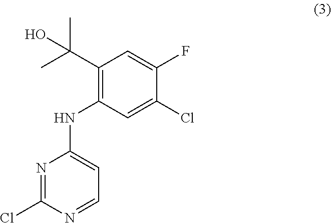 [SMILES: CC(C)(O)c1cc(F)c(Cl)cc1Nc1ccnc(Cl)n1]

with a compound of Formula (4):

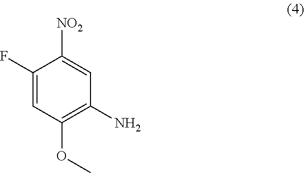

in the presence of an organic solvent and an organic acid. In some embodiments, the organic solvent is isopropanol and the organic acid is trifluoroacetic acid.

In some embodiments, the process for preparing Compound I comprises further step of (vi) preparing the compound of Formula (3) by contacting a compound of Formula (1) or a salt of the compound of Formula (1):

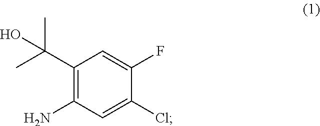

with a compound of Formula (8):

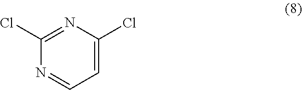

in the presence of an organic solvent and an organic base; and
(vii) crystallizing the mixture obtained in the step (vi) by addition of NH₄Cl aq. solution. In some embodiments, the salt of the compound of Formula (1) is selected from the group consisting of hydrochloric acid salt, methanesulfonic acid salt, sulfuric acid salt, phosphoric acid salt, maleic acid salt, fumaric acid salt, citric acid salt, succinic acid salt, L-malic acid salt, L-(+)-tartaric acid salt of the compound of Formula (1). In some embodiments, the organic solvent is isopropanol and the organic base is N,N-diisopropylethylamine.

Further provided herein is a process for preparing a compound of Formula (3), comprising a step of (i) contacting a compound of Formula (1) or a salt of the compound of Formula (1):

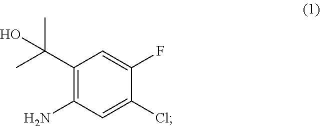

with a compound of Formula (8):

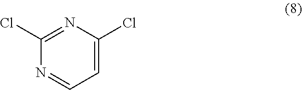

in the presence of an organic solvent and an organic base; and
(ii) crystallizing the mixture obtained in the step (i) by addition of NH₄Cl aq. solution. In some embodiments, the salt of the compound of Formula (1) is selected from the group consisting of: hydrochloric acid salt, methanesulfonic acid salt, sulfuric acid salt, phosphoric acid salt, maleic acid salt, fumaric acid salt, citric acid salt, succinic acid salt, L-malic acid salt, L-(+)-tartaric acid salt of the compound of Formula (1). In some embodiments, the organic solvent is isopropanol and the organic base is N,N-diisopropylethylamine.

Further provided herein is a re-crystallization process for preparing a Form B of Compound I, which comprises a step of dissolving Compound I in the Acetone/H₂O solution, adding a Form B crystal seed into the solution, leaving the solution to crystallize and isolating the Form B of Compound I.

### Pharmaceutical Compositions

In one aspect, the present disclosure also provides pharmaceutical compositions comprising one or more also such crystalline polymorphic forms as discussed above, and a pharmaceutically acceptable carrier.

The pharmaceutically acceptable carriers are conventional medicinal carriers in the art which can be prepared in a manner well known in the pharmaceutical art. In some embodiments, the compounds of the present disclosure may be admixed with pharmaceutically acceptable carrier for the preparation of pharmaceutical composition.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) alcohol, such as ethyl alcohol and propane alcohol; (20) phosphate buffer solutions; and (21) other non-toxic compatible substances employed in pharmaceutical formulations such as acetone.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The form of pharmaceutical compositions depends on a number of criteria, including, but not limited to, route of administration, extent of disease, or dose to be administered.

The pharmaceutical compositions can be formulated for oral, nasal, rectal, percutaneous, intravenous, or intramuscular administration. In accordance to the desired route of administration, the pharmaceutical compositions can be formulated in the form of tablets, capsule, pill, dragee, powder, granule, sachets, cachets, lozenges, suspensions, emulsions, solutions, syrups, aerosols (as a solid or in a liquid medium), spray, ointment, paste, cream, lotion, gel, patches, inhalant, or suppository.

The pharmaceutical compositions can be formulated to provide quick, sustained or delayed release of the active ingredient after administration to the patient by employing procedures known in the art. In some embodiments, the pharmaceutical composition is formulated in a sustained released form. In some embodiments, the prolonged period of time can be about 1 hour to 24 hours, 2 hours to 12 hours, 3 hours to 8 hours, 4 hours to 6 hours, 1 to 2 days or more. In certain embodiments, the prolonged period of time is at least about 4 hours, at least about 8 hours, at least about 12 hours, or at least about 24 hours. The pharmaceutical composition can be formulated in the form of tablet. For example, release rate of the active agent can not only be controlled by dissolution of the active agent in gastrointestinal fluid and subsequent diffusion out of the tablet or pills independent of pH, but can also be influenced by physical processes of disintegration and erosion of the tablet. In some embodiments, polymeric materials as disclosed in “Medical Applications of Controlled Release,” Langer and Wise (eds.), CRC Pres., Boca Raton, Florida (1974); “Controlled Drug Bioavailability,” Drug Product Design and Performance, Smolen and Ball (eds.), Wiley, New York (1984); Ranger and Peppas, 1983, J Macromol. Sci. Rev. Macromol Chem. 23:61; see also Levy et al., 1985, Science 228:190; During et al., 1989, Ann. Neurol. 25:351; Howard et al., 1989, J. Neurosurg. 71:105 can be used for sustained release. The above references are incorporated herein by reference in their entirety.

In certain embodiments, the pharmaceutical compositions comprise about 0.0001 mg to about 5000 mg of the compounds of the present disclosure (e.g. about 0.0001 mg to about 10 mg, about 0.001 mg to about 10 mg, about 0.01 mg to about 10 mg, about 0.1 mg to about 10 mg, about 1 mg to about 10 mg, about 5 mg to about 10 mg, about 5 mg to about 20 mg, about 5 mg to about 30 mg, about 5 mg to about 40 mg, about 5 mg to about 50 mg, about 10 mg to about 100 mg, about 20 mg to about 100 mg, about 30 mg to about 100 mg, about 40 mg to about 100 mg, about 50 mg to about 100 mg, about 50 mg to about 200 mg, about 50 mg to about 300 mg, about 50 mg to about 400 mg, about 50 mg to about 500 mg, about 100 mg to about 200 mg, about 100 mg to about 300 mg, about 100 mg to about 400 mg, about 100 mg to about 500 mg, about 200 mg to about 500 mg, about 300 mg to about 500 mg, about 400 mg to about 500 mg, about 500 mg to about 1000 mg, about 600 mg to about 1000 mg, about 700 mg to about 1000 mg, about 800 mg to about 1000 mg, about 900 mg to about 1000 mg, about 1000 mg to about 2000 mg, about 2000 mg to about 3000 mg, about 3000 mg to about 4000 mg, or about 4000 mg to about 5000 mg). Suitable dosages per subject per day can be from about 5 mg to about 500 mg, preferably about 5 mg to about 50 mg, about 50 mg to about 100 mg, or about 50 mg to about 500 mg.

In certain embodiments, the pharmaceutical compositions can be formulated in a unit dosage form, each dosage containing from about 0.0001 mg to about 10 mg, about 0.001 mg to about 10 mg, about 0.01 mg to about 10 mg, about 0.1 mg to about 10 mg, about 1 mg to about 10 mg, about 5 mg to about 10 mg, about 5 mg to about 20 mg, about 5 mg to about 30 mg, about 5 mg to about 40 mg, about 5 mg to about 50 mg, about 10 mg to about 100 mg, about 20 mg to about 100 mg, about 30 mg to about 100 mg, about 40 mg to about 100 mg, about 50 mg to about 100 mg, about 50 mg to about 200 mg, about 50 mg to about 300 mg, about 50 mg to about 400 mg, about 50 mg to about 500 mg, about 100 mg to about 200 mg, about 100 mg to about 300 mg, about 100 mg to about 400 mg, about 100 mg to about 500 mg, about 200 mg to about 500 mg, about 300 mg to about 500 mg, about 400 mg to about 500 mg, about 500 mg to about 1000 mg, about 600 mg to about 1000 mg, about 700 mg to about 1000 mg, about 800 mg to about 1000 mg, about 900 mg to about 1000 mg, about 1000 mg to about 2000 mg, about 2000 mg to about 3000 mg, about 3000 mg to about 4000 mg, or about 4000 mg to about 5000 mg of the compounds of the present disclosure. The term “unit dosage forms” refers to physically discrete units suitable as unitary dosages for human subjects and other mammals, each unit containing a predetermined quantity of active material calculated to produce the desired therapeutic effect, in association with a suitable pharmaceutical carrier.

In some embodiments, the pharmaceutical compositions comprise one or more pharmaceutical salts and/or polymorphs of the present disclosure as a first active ingredient, and further comprise a second active ingredient. The second active ingredient can be any anti-cancer agent known in the art, for examples, cell signal transduction inhibitors, cell signal transduction inhibitors, alkylating agents, topoisomerase inhibitors, immunotherapeutic agents, mitosis inhibitors, antihormonal agents, chemotherapy drugs, EGFR inhibitors, CTLA-4 inhibitors, MEK inhibitors, PD-L1 inhibitors; OX40 agonists, and the like. Representative examples of the anti-cancer agents for treating cancers or tumors may include, but are not limited to, sorafenib, sunitinib, dasatinib, vorinostat, temsirolimus, everolimus, pazopanib, trastuzumab, ado-trastuzumab emtansine, pertuzumab, bevacizumab, cetuximab, ranibizumab, pegaptanib, panitumumab, tremelimumab, pembrolizumab, nivolumab, ipilimumab, atezolizumab, avelumab, durvalumab, crizotinib, ruxolitinib, paclitaxel, vincristine, vinblastine, cisplatin, carboplatin, gemcitabine, tamoxifen, raloxifene, cyclophosphamide, chromabucil, carmustine, methotrexate, fluorouracil, actinomycin, doxorubicin, epirubicin, anthracycline, bleomycin, mitomycin-C, irinotecan, topotecan, teniposide interleukin, interferon, and the like. In some embodiments, the second active agent is one or more of bevacizumab, pembrolizumab, nivolumab, ipilimumab, atezolizumab, avelumab, durvalumab, crizotinib.

### Uses and Method for Treatment

In one aspect, the crystalline form, pharmaceutical salts, or pharmaceutical composition provided herein are for use as a medicament for inhibiting ErbB (e.g., EGFR, Her2, Her3 or Her4) or BTK. In another aspect, the present disclosure provides use of the crystalline form, pharmaceutical salt, or pharmaceutical composition of the present disclosure in the manufacture of medicaments for treating diseases associated with ErbB or BTK.

In one aspect, the present disclosure provides a method of inhibiting ErbB or BTK by using one or more crystalline form, pharmaceutical salt, or pharmaceutical composition provided herein.

In another aspect, the present disclosure also provides a method of inhibiting ErbB or BTK by using one or more crystalline form, pharmaceutical salts, or pharmaceutical composition provided herein.

In yet another aspect, the present disclosure provides a method of treating an ErbB (including, for example, EGFR or Her2, especially ErbB mutant), associated diseases or BTK associated diseases in a subject, comprising administering to the subject an effective amount of one or more crystalline form, pharmaceutical salts, or pharmaceutical composition provided herein.

In some embodiments, the subject is a warm blooded-animal such as man.

In some embodiments, an ErbB associated diseases or BTK associated diseases is cancer, autoimmune diseases, or inflammation. In some embodiments, the ErbB associated diseases is cancer. In certain embodiments, the ErbB associated diseases are diseases associated with the mutant ErbB. In some embodiments, the mutant ErbB is mutant EGFR. In some embodiments, the mutant ErbB is mutant Her2. In certain embodiments, the diseases associated with ErbB are diseases associated with mutant ErbB, including cancers. In some embodiments, the BTK associated disease is cancer or an autoimmune disease.

In some embodiments, the cancers include but are not limited to, leukemia, glioblastoma, melanoma, chondrosarcoma, cholangiocarcinoma, osteosarcoma, lymphoma, lung cancer, adenoma, myeloma, hepatocellular carcinoma, adrenocortical carcinoma, pancreatic cancer, breast cancer, bladder cancer, prostate cancer, liver cancer, gastric cancer, colon cancer, colorectal cancer, ovarian cancer, cervical cancer, brain cancer, esophageal cancer, bone cancer, testicular cancer, skin cancer, kidney cancers, mesothelioma, neuroblastoma, thyroid cancer, head and neck cancers, esophageal cancers, eye cancers, prostate cancer, nasopharyngeal cancer, or oral cancer. In some embodiments, the cancers are lung cancer, breast cancer, ovarian cancer, bladder cancer, or glioblastoma. In some embodiments, the cancer is lung cancer (e.g., non-small cell lung cancer, small cell lung cancer, adenocarcinoma, squamous cell lung cancer and large cell lung cancer). In some embodiments, the cancer is lymphoma or leukemia. In some embodiments, the cancer is metastatic lung cancer. In some embodiment, the cancer is cancer with one or more ErbB mutations (e.g., point mutations, deletion mutations, insertion mutations, activating mutations, or drug resistant mutations of EGFR or Her2). In some embodiments, the autoimmune disease is rheumatoid arthritis, systemic lupus erythematosus or Sjogren's syndrome.

In some embodiments, the ErbB is EGFR or Her2, preferably is mutant EGFR or mutant Her2. In some embodiments, the mutant EGFR selected from EGFR D761_E762insEAFQ, EGFR A763_Y764insHH, EGFR M766_A767instAI, EGFR A767_V769dupASV, EGFR A767_S768insTLA, EGFR S768_D770 dupSVD, EGFR S768_V769insVAS, EGFR S768_V769insAWT, EGFR V769_D770insASV, EGFR V769_D770insGV, EGFR V769_D770insCV, EGFR V769_D770insDNV, EGFR V769 D770insGSV, EGFR V769 D770insGVV, EGFR V769 D770insMASVD, EGFR D770_N771insSVD, EGFR D770_N771insNPG, EGFR D770_N771insAPW, EGFR D770_N771insD, EGFR D770_N771insDG, EGFR D770_N771insG, EGFR D770_N771insGL, EGFR D770_N771insN, EGFR D770_N771insNPH, EGFR D770_N771insSVP, EGFR D770_N771insSVQ, EGFR D770_N771insMATP, EGFR delD770insGY, EGFR N771_P772insH, EGFR N771_P772insN, EGFR N771_H773dupNPH, EGFR delN771insGY, EGFR delN771insGF, EGFR P772_H773insPR, EGFR P772_H773insYNP, EGFR P772_H773insX, EGFR P772_H773insDPH, EGFR P772_H773insDNP, EGFR P772_H773insQV, EGFR P772_H773insTPH, EGFR P772_H773insN, EGFR P772_H773insV, EGFR H773 V774insNPH, EGFR H773 V774insH, EGFR H773 V774insPH, EGFR H773 V774insGNPH, EGFR H773 V774dupHV, EGFR H773_V774insG, EGFR H773 V774insGH, EGFR V774 C775insHV, EGFR exon19 deletion, EGFR L858R, EGFR T790M, EGFR L858R/T790M, EGFR exon 19 deletion/T790M, EGFR S768I, EGFR G719S, EGFR G719A, EGFR G719C, EGFR E709A/G719S, EGFR E709A/G719A, EGFR E709A/G719C, and EGFR L861Q. In some embodiments, the mutant Her2 is selected from the group consisting of Her2 A775_G776insYVMA, Her2 delG776insVC, Her2 V777 G778insCG and Her2 P780 Y781insGSP.

The crystalline form, pharmaceutical salt, or pharmaceutical composition in the present disclosure can be used in the prevention or treatment of the onset or development of any of the diseases or conditions associated with ErbB/BTK (expression or activities) in mammals especially in human. In some embodiments, the crystalline form, pharmaceutical salt, or pharmaceutical composition in the present disclosure can be used in the prevention or treatment of the onset or development of any of the diseases or conditions associated with mutant ErbB in mammals especially in human. In such situation, the present disclosure also provides a method of screening patient suitable for treating with the compounds or pharmaceutical composition of the present disclosure alone or combined with other ingredients (e.g. a second active ingredient, e.g. anti-cancer agent). The method includes sequencing the tumor samples from patients and detecting the accumulation of ErbB (e.g., EGFR or Her2) or BTK in the patient or detecting the mutations status of ErbB (e.g., EGFR or Her2) or BTK in the patient.

In some embodiments, the one or more crystalline form, pharmaceutical salts, or pharmaceutical composition provided herein is administered via a parenteral route or a non-parenteral route. In some embodiments, the one or more crystalline form, pharmaceutical salts, or pharmaceutical composition provided herein is administered orally, enterally, buccally, nasally, intranasally, transmucosally, epidermally, transdermally, dermally, ophthalmically, pulmonary, sublingually, rectally, vaginally, topically, subcutaneously, intravenously, intramuscularly, intraarterially, intrathecally, intracapsularly, intraorbitally, intracardiacally, intradermally, intraperitoneally, transtracheally, subcuticularly, intra-articularly, subcapsularly, subarachnoidly, intraspinally, or intrasternally.

The crystalline forms or pharmaceutical salts provided herein can be administrated in pure form, or in the form of pharmaceutically compositions of the present disclosure. In some embodiments, the one or more crystalline form, pharmaceutical salts, or pharmaceutical composition provided herein is used in combination with a second active ingredient, preferably an anti-cancer agent. In some embodiments, the crystalline form, pharmaceutical salts, or pharmaceutical composition provided herein can be administered to a subject in need concurrently or sequentially in a combination with a second active ingredient (e.g. one or more anti-cancer agent(s) known in the art). In some embodiments, the administration is conducted once a day, twice a day, three times a day, or once every two days, once every three days, once every four days, once every five days, once every six days, once a week.

In some embodiments, the one or more crystalline form, pharmaceutical salts, or pharmaceutical composition provided herein is administered orally. For oral administration, any dose is appropriate that achieves the desired goals. In some embodiments, suitable daily dosages are between about 0.001-5000 mg, preferably between 0.1 mg and 5 g, more preferably between 5 mg and 1 g, more preferably between 10 mg and 500 mg, and the administration is conducted once a day, twice a day, three times a day, every day, or 3-5 days a week. In some embodiments, the dose of the one or more compounds, pharmaceutically acceptable salts, esters, hydrates, solvates or stereoisomers thereof or the pharmaceutical composition provided herein ranges between about 0.0001 mg, preferably, 0.001 mg, 0.01 mg, 0.1 mg, 1 mg, 10 mg, 50 mg, 100 mg, 200 mg, 250 mg, 500 mg, 750 mg, 1000 mg, 2000 mg, 3000 mg, 4000 mg or up to about 5000 mg per day.

### Examples

The following abbreviations have the definitions set forth below:

| | |
|---|---|
| ¹³C NMR | Carbon-13 Nuclear Magnetic Resonance |
| | Spectroscopy |
| ¹H NMR | Proton Nuclear Magnetic Resonance |
| | Spectroscopy |
| a.q. | aqueous |
| Acetone | Propanone |
| CDCl₃ | deuterated chloroform |
| CH₂Cl₂ | Dichloromethane |
| CH₃CN or MeCN or | Acetonitrile |
| ACN | |
| d₆-DMSO | Deuterated dimethyl sulfoxide |
| DIEA or DIPEA | N,N,-diisopropylethylamine |
| d-MeOH or CD₃OH | Deuterated methanol |
| DMF | dimethyl formamide |
| DMSO | dimethyl sulfoxide |
| DSC | Differential Scanning Calorimetry |
| DVS | Dynamic Vapor Sorption |
| EtOAc or EA | ethyl acetate |
| EtOH | Ethanol |
| HCl | hydrochloric acid |
| HPLC | High-performance liquid chromatography |
| IPA | isopropanol |
| K₂CO₃ | potassium carbonate |
| Kg | Kilogram(s) |
| MeOH | Methanol |
| Mol. Eq. | molar equivalent |
| MTBE | methyl tert-butyl ether |
| Na₂SO₄ | sodium sulfate |
| NaCl | Sodium chloride |
| n-BuOH | 1-butanol |
| NH₄Cl | ammonium chloride |
| ORTEP | Oak Ridge Thermal-Ellipsoid Plot |
| Pd/C | palladium on carbon |
| Pt/C | platinum on carbon |
| rel. vol. | Relative volume |
| Silica gel | Silica gel |
| TEA or Et₃N | Triethylamine |
| TFA | trifluoroacetic acid |
| TGA | Thermal Gravimetric Analysis |
| THF | Tetrahydrofuran |
| v/v | Volume/volume |
| XRPD | X-Ray Powder Diffraction |

For clarity, below table summarized the compound identifier, chemical name, and structure used interchangeably throughout this application with respect to each compound discussed.

| COMPOUND | | |
|---|---|---|
| IDENTIFIER | NAME | STRUCTURE |
| (2) | 2-(2-amino-4-chloro-5- fluorophenyl)propan-2-ol | 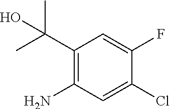 |
| (3) | 2-(4-chloro-2-((2- chloropyrimidin-4-yl)amino)-5- fluorophenyl)propan-2-ol | 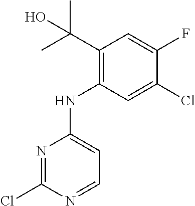 [SMILES: CC(C)(O)c1cc(F)c(Cl)cc1Nc1ccnc(Cl)n1] |
| (4) | 4-fluoro-2-methoxy-5-nitroaniline | 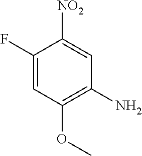 |
| (5) | 2-(4-chloro-5-fluoro-2-((2-((4- fluoro-2-methoxy-5- nitrophenyl)amino)pyrimidin-4- yl)amino)phenyl)propan-2-ol | 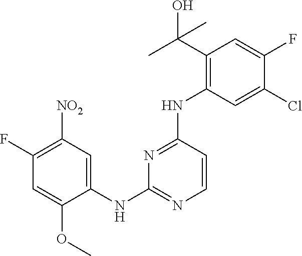 [SMILES: COc1cc(F)c([N+](=O)[O-])cc1Nc1nccc(Nc2cc(Cl)c(F)cc2C(C)(C)O)n1] |
| (6) | (R)-2-(4-chloro-2-((2-((4-(3- (dimethylamino)pyrrolidin-1-yl)- 2-methoxy-5- nitrophenyl)amino)pyrimidin-4- yl)amino)-5-fluorophenyl)propan- 2-ol | 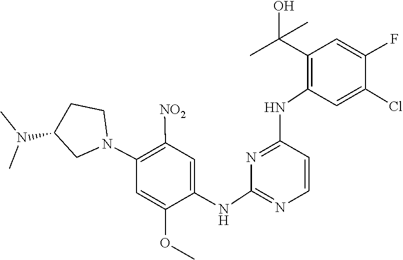 [SMILES: COc1cc(N2CC[C@@H](N(C)C)C2)c([N+](=O)[O-])cc1Nc1nccc(Nc2cc(Cl)c(F)cc2C(C)(C)O)n1] |
| (7) | (R)-2-(2-((2-((5-amino-4-(3- (dimethylamino)pyrrolidin-1-yl)- 2- methoxyphenyl)amino)pyrimidin- 4-yl)amino)-4-chloro-5- fluorophenyl)propan-2-ol | 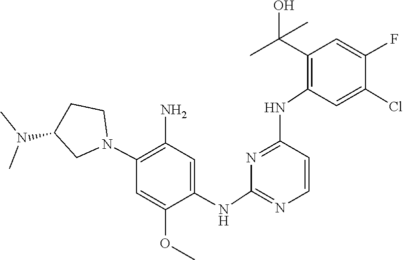 [SMILES: COc1cc(N2CC[C@@H](N(C)C)C2)c(N)cc1Nc1nccc(Nc2cc(Cl)c(F)cc2C(C)(C)O)n1] |
| (8) | 2,4-dichloropyrimidine | 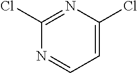 |
| (9) | 2-(2-amino-4-chloro-5- fluorophenyl)propan-2-ol hydrogen chloride | 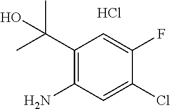 |
| (10) | (R)-N,N-dimethylpyrrolidin-3- amine | 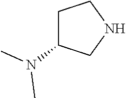 |
| (11) | (R)-N,N-dimethylpyrrolidin-3- amine hydrogen chloride | 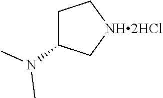 |
| (12) | (R)-3-chloro-N-(5-((4-((5-chloro- 4-fluoro-2-(2-hydroxypropan-2- yl)phenyl)amino)pyrimidin-2- yl)amino)-2-(3- (dimethylamino)pyrrolidin-1-yl)- 4-methoxyphenyl)propanamide | 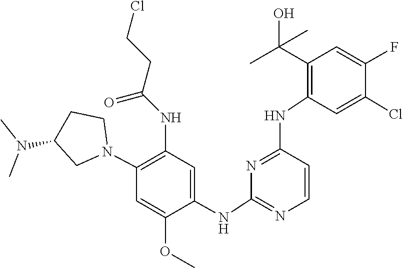 [SMILES: COc1cc(N2CC[C@@H](N(C)C)C2)c(NC(=O)CCCl)cc1Nc1nccc(Nc2cc(Cl)c(F)cc2C(C)(C)O)n1] |
| Compound I | (R)-N-(5-((4-((5-chloro-4-fluoro- 2-(2-hydroxypropan-2- yl)phenyl)amino)pyrimidin-2- yl)amino)-2-(3- (dimethylamino)pyrrolidin-1-yl)- 4-methoxyphenyl)acrylamide | 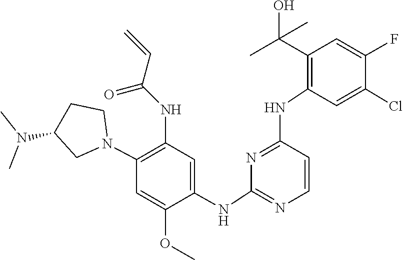 [SMILES: C=CC(=O)Nc1cc(Nc2nccc(Nc3cc(Cl)c(F)cc3C(C)(C)O)n2)c(OC)cc1N1CC[C@@H](N(C)C)C1] |

### Example 1. Analysis Method

### ¹H NMR Analysis

¹H NMR was performed using Bruker AVANCE III, Bruker Ultrashield 400 or Bruker Advance 300 equipped with automated sampler (B-ACS 120).

### Powder X-Ray Diffraction (XRPD)

Solid samples were examined using D8 advance or D2 X-ray diffractometer (Bruker). The system was equipped with LynxEye detector. Samples were scanned from 3 to 40° 20, at a step of 0.02° 20. The tube voltage and current were 40 KV and 40 mA (D8 ADVANCE), 30 KV and 10 mA, respectively.

### Polarizing Microscope Analysis (PLM)

PLM analysis was conducted with a Polarizing Microscope ECLIPSE LV100POL (Nikon, JPN). Put the sample on a piece of glass slide, dispersed with cedar oil and observed with suitable magnification.

### Thermogravimetric Analysis (TGA)

TGA was carried out on TGA Q5000IR, Q500, Discovery TGA 55 (TA Instruments, US) or Mettler Toledo TGA 2. The sample was placed in an open tarred aluminum pan, automatically weighed, and inserted into the TGA furnace. The sample was heated at 10° C./min to the final temperature.

### Differential Scanning Calorimeter (DSC)

DSC analysis was conducted with DSC Q2000, Q200, Discovery DSC 250 (TA Instruments, US) or Mettler Toledo DSC 3+. A weighed sample was placed into a DSC pinhole pan, and the weight was accurately recorded. The sample was heated at 10° C./min to the final temperature.

### Dynamic Moisture Sorption Analysis (DVS)

DVS was determined using DVS Advantage-1 or Intrinsic (SMS, UK). The sample was tested at a targeted RH of 10 to 90% full cycle in step mode. The analysis was performed in 10% RH increments. Equilibrium:60 min RH (%) measurement points: First cycle: 0, 10, 20, 30, 40, 50, 60, 70, 80, 90. Second cycle: 90, 80, 70, 60, 50, 40, 30, 20, 10, 0.

### Example 2. Procedures for the Preparation of (R)—N-(5-((4-((5-chloro-4-fluoro-2-(2-hydroxypropan-2-yl)phenyl)amino)pyrimidin-2-yl)amino)-2-(3-(dimethylamino)pyrrolidin-1-yl)-4-methoxyphenyl)acrylamide (Compound I Free Base)

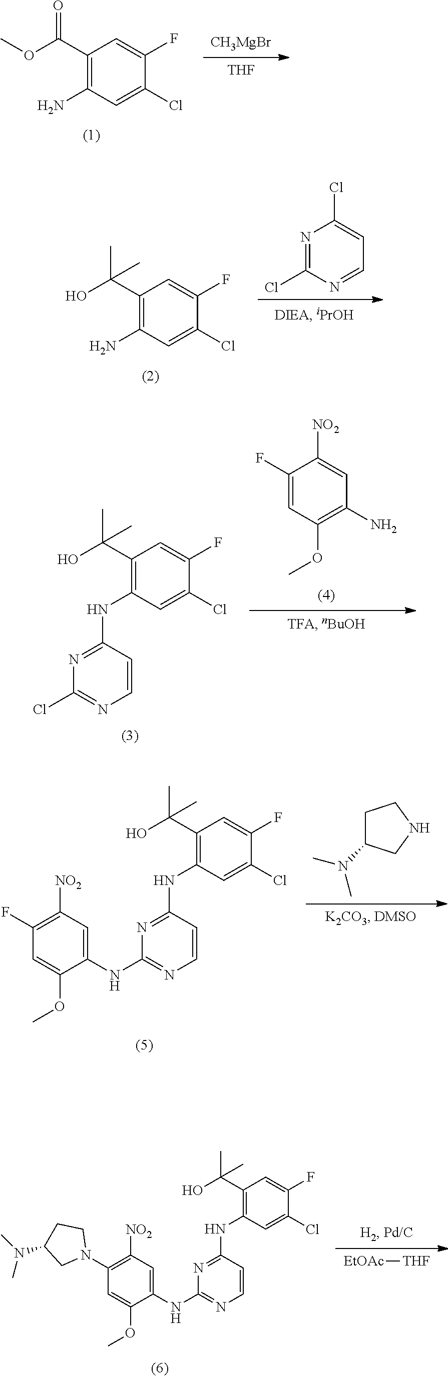

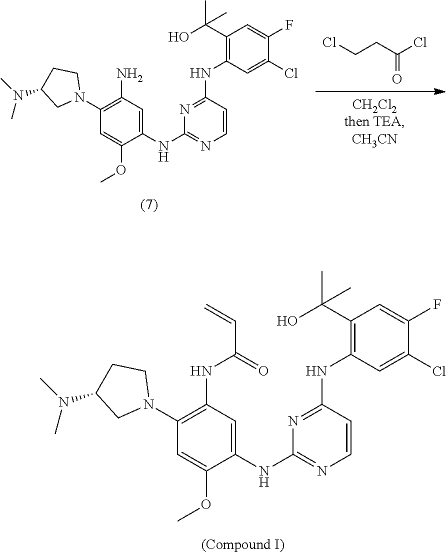 [SMILES: COc1cc(N2CC[C@@H](N(C)C)C2)c(N)cc1Nc1nccc(Nc2cc(Cl)c(F)cc2C(C)(C)O)n1 ; O=C(Cl)CCCl ; "CH2Cl2then TEA,CH3CN" ; C=CC(=O)Nc1cc(Nc2nccc(Nc3cc(Cl)c(F)cc3C(C)(C)O)n2)c(OC)cc1N1CC[C@@H](N(C)C)C1]

### Procedure for the Preparation of Compound (2)

To a solution of methyl 2-amino-4-chloro-5-fluorobenzoate (1) (12.0 g, 58.9 mmol) in THF (200 mL) was added CH₃MgBr (99 mL, 3M in ether, 294.7 mmol) at 0˜5° C. The mixture was stirred at 12-17° C. for 1.5 h. The reaction mixture was quenched by the addition of aq. NH₄Cl (100 mL), then extracted with EtOAc (3×100 mL). The organic layers were washed with brine (3×100 mL), and concentrated under reduced pressure to afford compound (2) (11.5 g, 96%) as light yellow oil.

LCMS: Rt=3.283 min in 10-80CD_7MIN 220&254 chromatography (XBrige Shield RP18 2.1*50 mm), MS (ESI) m/z 186.1 [M−OH]⁺.

¹H NMR (CDCl₃, 400 MHz): δ (ppm) 6.90 (d, J=10.8 Hz, 1H), 6.62 (d, J=6.8 Hz, 1H), 1.63 (s, 6H).

¹³C NMR (d₆-DMSO, 101 MHz) δ (ppm) 149.7, 147.4, 144.3, 131.3, 131.2, 116.9, 116.7, 115.7, 113.6, 113.4, 71.6, 28.7.

### Procedure for the Preparation of Compound (3)

To a solution of compound (2) (11.5 g, 56.5 mmol) and DIEA (14.6 g, 112.9 mmol) in isopropanol (200 mL) was added 2,4-dichloropyrimidine (10.1 g, 67.8 mmol). The resulting yellow mixture was heated at 90° C. for 60 h. The reaction mixture was concentrated in vacuum to give the crude product, which was purified by column chromatography on silica gel (30-43% EtOAc in petroleum ether) to give compound (3) (12.0 g, 67%) as a white solid.

LCMS: t_{R}=0.850 min in 5-95AB_220&254.1 cm chromatography (Xtimate C18 2.1*30 mm), MS (ESI) m/z=315.9 [M+H]⁺.

¹H NMR (CDCl₃, 400 MHz): δ (ppm) 9.17 (br s, 1H), 8.15 (d, J=5.6 Hz, 1H), 7.95 (d, J=6.8 Hz, 1H), 7.12 (d, J=10.0 Hz, 1H), 6.58 (d, J=6.0 Hz, 1H), 2.35 (s, 1H), 1.65 (s, 6H).

¹³C NMR (d₆-DMSO, 101 MHz) δ (ppm) 161.9, 159.4, 157.8, 155.2, 152.8, 142.7, 132.8, 132.7, 126.6, 117.4, 117.2, 114.6, 114.4, 105.3, 71.7, 29.7.

### Procedure for the Preparation of Compound (5)

To a solution of compound (3) (12.0 g, 38.0 mmol) and 4-fluoro-2-methoxy-5-nitroaniline (4) (7.44 g, 40.0 mmol) in ⁿBuOH (160 mL) was added TFA (16 mL). The resulting orange mixture was heated at 50° C. for 15 h. The reaction mixture changed from orange to pale yellow and solid precipitated out, additional 300 mg of 4-fluoro-2-methoxy-5-nitroaniline was added and the reaction mixture was heated at 50° C. for another 4 h. The reaction mixture was filtered, the filter cake was washed with EtOAc/petroleum ether=1/1 (25 mL×3) and EtOAc (25 mL×3), then dried in vacuum to give compound (5) (15.2 g, 86%) as a grey solid.

LCMS: t_{R}=0.776 min in 5-95AB_220&254.1 cm chromatography (Xtimate C18 2.1*30 mm), MS (ESI) m/z=466.0 [M+H]⁺.

¹H NMR (CDCl₃, 400 MHz) δ (ppm) 8.52 (d, J=8.0 Hz, 1H), 7.96 (d, J=6.8 Hz, 1H), 7.84 (d, J=7.2 Hz, 1H), 7.32 (d, J=10.8 Hz, 1H), 7.20 (d, J=12.8 Hz, 1H), 6.47 (d, J=6.8 Hz, 1H), 4.00 (s, 3H), 1.59 (s, 6H).

¹³C NMR (d₆-DMSO, 101 MHz) δ (ppm) 161.6, 156.2, 153.7, 152.6, 143.9, 143.5, 131.0, 128.6, 128.1, 121.9, 117.3, 117.1, 114.6, 114.4, 102.1, 101.9, 100.0, 71.5, 57.5, 29.9.

### Procedure for the Preparation of Compound (6)

To a solution of compound (5) (5.0 g, 10.7 mmol) and K₂CO₃ (5.9 g, 42.9 mmol) in DMSO (50 mL) was added (R)—N,N-dimethylpyrrolidin-3-amine (2.6 g, HCl salt, 14.0 mmol). The resulting mixture was stirred at 50° C. for 12 h while the color was changed from pale yellow to deep yellow. The reaction mixture was poured into ice water (500 mL) with stirring and yellow solid was precipitated. The precipitated solid was collected by filtration and then dissolved into CH₂Cl₂ (500 mL), dried over anhydrous Na₂SO₄ and concentrated under reduced pressure to give compound (6) (5.6 g, 93%) as yellow solid.

LCMS: Rt=0.676 min in 5-95AB_220&254.1 cm chromatography (MK RP-18e 25-2 mm), MS (ESI) m/z=560.1 [M+H]⁺.

¹H NMR (CDCl₃, 400 MHz) δ (ppm) 9.00 (s, 1H), 8.91 (s, 1H), 8.09 (d, J=5.8 Hz, 1H), 7.93 (d, J=7.0 Hz, 1H), 7.19 (s, 1H), 7.11 (d, J=10.5 Hz, 1H), 6.31 (s, 1H), 6.18 (d, J=5.8 Hz, 1H), 5.31 (s, 1H), 3.94 (s, 3H), 3.55 (td, J=10.1, 6.4 Hz, 1H), 3.31-3.39 (m, 1H), 3.10-3.22 (m, 2H), 2.81 (br s, 1H), 2.30 (s, 6H), 2.15-2.25 (m, 1H), 1.83-1.98 (m, 1H), 1.67 (s, 6H).

¹³C NMR (d₆-DMSO, 101 MHz) δ (ppm) 159.0, 158.9, 155.7, 154.9, 152.5, 150.1, 140.4, 137.7, 133.7, 127.4, 122.8, 119.8, 117.6, 116.0, 115.8, 112.9, 112.7, 97.0, 96.5, 71.0, 63.7, 55.0, 48.4, 42.8, 28.5.

### Procedure for the Preparation of Compound (7)

To a solution of compound (6) (5.6 g, 10.0 mmol) in EtOAc (100 mL) and THF (50 mL) was added Pd/C (1.2 g). The resulting mixture was purged and degassed with H₂ for 3 times, then stirred at 11-18° C. under H₂ (hydrogen balloon, 15 Psi) for 16 h. The reaction mixture was filtered and concentrated under reduced pressure to give compound (7) (5.0 g, 94%) as light yellow solid.

LCMS: Rt=0.660 min in 5-95AB_1.5 min_220&254 chromatography (MK RP18e 25-2 mm), MS (ESI) m/z=530.1 [M+H]⁺.

¹H NMR (CDCl₃, 400 MHz) δ (ppm) 8.80 (s, 1H), 8.15 (d, J=7.3 Hz, 1H), 8.04 (d, J=5.5 Hz, 1H), 7.87 (s, 1H), 7.43 (s, 1H), 7.09 (d, J=10.5 Hz, 1H), 6.67 (s, 1H), 6.06 (d, J=5.5 Hz, 1H), 3.82 (s, 3H), 3.24-3.13 (m, 2H), 3.07-2.96 (m, 2H), 2.91-2.83 (m, 1H), 2.28 (s, 6H), 2.18-2.08 (m, 1H), 1.90-1.85 (m, 1H), 1.66 (s, 6H).

¹³C NMR (d₆-DMSO, 101 MHz) δ (ppm) 160.1, 156.8, 153.7, 151.3, 142.3, 139.1, 135.4, 134.9, 131.4, 124.2, 123.9, 117.2, 117.0, 114.1, 113.9, 110.0, 103.5, 97.5, 72.1, 64.9, 56.3, 54.5, 49.8, 29.6, 28.6.

### Procedure for the Preparation of Compound I

Step 1: To a solution of compound (7) (5.0 g, 9.43 mmol) in CH₂C2 (150 mL) was added 3-chloropropanoyl chloride (1.3 g, 10.37 mmol) in ice water bath. The resulting mixture was stirred at 0-5° C. for 30 min (little un-dissolved oil was precipitated out). The reaction mixture was poured into saturated NaHCO₃ (50 mL) and stirred at 12-17° C. for 2 h, and extracted with CH₂Cl₂ (150 mL×2). The combined organic layers were dried over Na₂SO₄ and concentrated under reduced pressure to give the crude residue, which was purified by column chromatography on silica gel (3% MeOH in CH₂Cl₂) to give a light yellow solid (3.4 g, 58% yield).

LCMS: Rt=1.547 min in 10-80AB_4 min_220&254 chromatography (Xtimate C18 2.1*30 mm), MS (ESI) m/z=620.0 [M+H]⁺.

¹H NMR (CDCl₃, 400 MHz) δ (ppm) 9.58 (s, 1H), 9.31 (s, 1H), 8.56 (br s, 1H), 8.10 (d, J=5.8 Hz, 1H), 7.62-7.45 (m, 2H), 7.15 (d, J=10.5 Hz, 1H), 6.76 (s, 1H), 6.34 (d, J=5.8 Hz, 1H), 3.90 (t, J=6.3 Hz, 2H), 3.86 (s, 3H), 3.16-3.03 (m, 4H), 2.90 (br s, 3H), 2.32 (br s, 6H), 2.19 (br dd, J=6.3, 12.3 Hz, 1H), 1.98 (br s, 1H), 1.75-1.68 (m, 6H).

Step 2: To a solution of the yellow solid from step 1 (3.4 g, 5.48 mmol) in CH₃CN (70 mL) was added TEA (2.2 g, 21.92 mmol). The resulting mixture was stirred at 80° C. for 12 h. The reaction mixture was concentrated under reduced pressure to remove about 35 mL CH₃CN, and then poured onto 500 mL H₂O and stirred for additional 30 min. The mixture was filtered, the filter cake was collected and then lyophilized to give the title product Compound I (2.64 g, 82%) as a white solid.

LCMS: Rt=1.471 min in 10-80AB 4 min 220&254 chromatography (Xtimate C18 2.1*30 mm), MS (ESI) m/z=584.0 [M+H]⁺.

¹H NMR (CDCl₃, 400 MHz) δ (ppm) 9.67 (s, 1H), 9.44 (s, 1H), 8.55 (br s, 1H), 8.10 (d, J=6.0 Hz, 1H), 7.52 (br d, J=7.0 Hz, 1H), 7.48 (s, 1H), 7.15 (d, J=10.8 Hz, 1H), 6.76 (s, 1H), 6.42-6.28 (m, 3H), 5.82-5.75 (m, 1H), 5.66 (br s, 1H), 3.86 (s, 3H), 3.14-3.02 (m, 4H), 2.96-2.86 (m, 1H), 2.30 (s, 6H), 2.23-2.12 (m, 1H), 2.00-1.90 (m, 1H), 1.73 (s, 6H).

¹³C NMR (d₆-DMSO, 101 MHz) δ (ppm) 163.5, 160.5, 159.9, 156.8, 153.5, 151.1, 149.9, 141.5, 138.5, 135.0, 132.0, 125.7, 123.7, 123.4, 119.9, 117.9, 117.2, 117.0, 114.0, 113.8, 99.5, 97.5, 72.2, 65.2, 55.6, 49.3, 43.9, 29.6.

### Example 3. Scale-Up Manufacturing Processes of (R)—N-(5-((4-((5-chloro-4-fluoro-2-(2-hydroxypropan-2-yl)phenyl)amino)pyrimidin-2-yl)amino)-2-(3-(dimethylamino)pyrrolidin-1-yl)-4-methoxyphenyl)acrylamide (Compound I Free Base)

Procedure for the preparation of Compound (3)

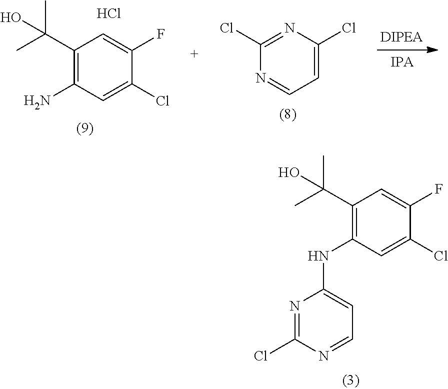 [SMILES: CC(C)(O)c1cc(F)c(Cl)cc1N ; Cl ; Clc1ccnc(Cl)n1 ; CC(C)(O)c1cc(F)c(Cl)cc1Nc1ccnc(Cl)n1]

Isopropanol (249.5 kg), DIPEA (118.6 kg) and compound (8) (78.1 kg) were charged into a reactor. Compound (9) (62.8 kg) was charged in the end under N2 protection. The mixture was adjusted to 78° C. (75-82° C.) and stirred for 18h until reaction was deemed complete. The reaction mixture was adjusted to 25° C., and 15 wt % NH₄Cl aqueous solution (1093 kg) was charged dropwise. The resulting mixture was stirred for 3 h at 25° C. and filtered. The cake was washed with purified water (95.0 kg*2), then the wet cake was slurried in IPA (252.2 kg) at 60° C. for 4 h. The slurry mixture was adjusted to 15° C. and stirred for 3 h. Then the slurry mixture was filtered and the wet cake was washed with IPA (100 kg). The wet cake was dried at 45° C. for 20 h, 67.06 kg of Compound (3) was obtained with 99.4% HPLC purity, 79.2% isolated yield by assay. ¹H NMR (DMSO-d₆, 400 MHz), 1.47 (6H, s), 6.03 (1H, s), 6.74˜6.76 (1H, d), 7.45˜7.48 (1H, d), 7.90-7.92 (1H, d), 8.16˜8.18 (1H, d), 9.87 (1H, s).

### Procedure for the Preparation of Compound (5)

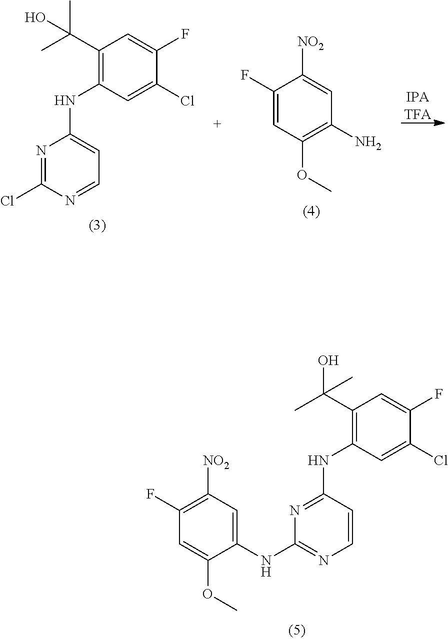 [SMILES: CC(C)(O)c1cc(F)c(Cl)cc1Nc1ccnc(Cl)n1 ; COc1cc(F)c([N+](=O)[O-])cc1N ; COc1cc(F)c([N+](=O)[O-])cc1Nc1nccc(Nc2cc(Cl)c(F)cc2C(C)(C)O)n1 ; "IPATFA"]

THF (189 kg), compound (3) (62.9 kg) and compound (4) (39.1 kg) were charged into a reactor, stirred at 25° C. and TFA (10.8 kg) was charged in 2h dropwise. The reaction system was adjusted to 50-60° C. and stirred for 24˜28h until reaction was deemed complete. The reaction system was adjusted to 20-30° C. and stirred for 2˜4 h, then filtered. The wet cake was washed with IPA (154 kg) and dried at 45° C. for 27h. 94.22 kg of Compound (5) was obtained with 99.3% HPLC purity, 93.4% isolated yield by assay. ¹H NMR (DMSO-d₆, 400 MHz), 1.47 (6H, s), 3.95 (3H, s), 6.66˜6.68 (1H, d), 7.38˜7.41 (1H, d), 7.46-7.49 (1H, d), 7.67˜7.69 (1H, d), 8.12˜8.13 (1H, d), 8.37˜8.39 (1H, d), 10.16 (1H, s), 10.80 (1H, s).

### Procedure for the Preparation of Compound (6)

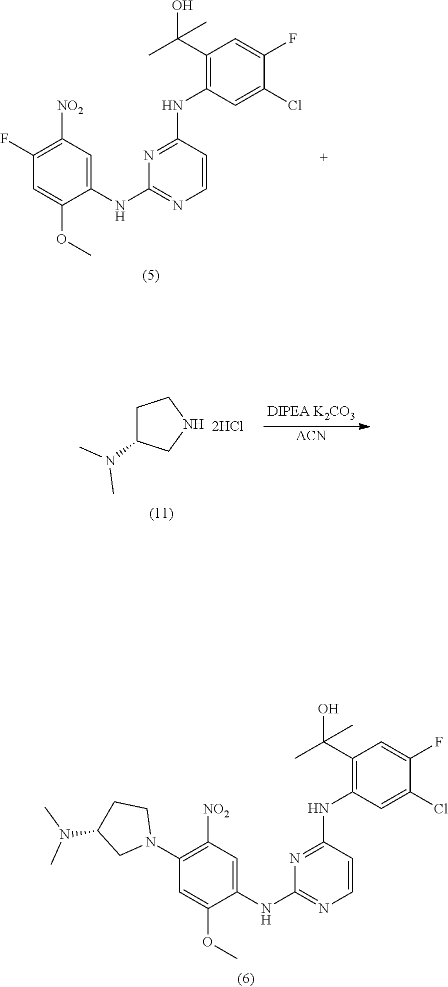

Acetonitrile (328 kg), K₂CO₃ (50.4 kg), compound (11) (44.8 kg) and DIPEA (140 kg) were charged into a reactor at 15-25° C., compound (5) (assay corrected 82.0 kg) was charged into the reactor. The reaction system was adjusted to 75-82° C. and stirred for 20˜30h until reaction was deemed complete. The reaction mixture was adjusted to 35-45° C. The purified water (492 kg) was added dropwise into the reaction mixture which was stirred for 4-6h. The reaction mixture was adjusted to 15-25° C., stirred for 3-5h and filtered. The wet cake was washed with ACN/H₂O (148 kg) and with H₂O (164 kg). H₂O (576 kg) was charged into the reactor followed by the wet cake. The mixture was stirred at 15-25° C. for 4 h and filtered. The wet cake was washed with H₂O (164 kg) and with ACN (148 kg). The wet cake was dried at 45° C. for 20h. 88.91 kg of Compound (6) was obtained with 99.8% HPLC purity, 89.2% isolated yield by assay. ¹H NMR (DMSO-d₆, 400 MHz), 1.65 (6H, s), 1.72˜1.82 (1H, m), 2.07˜2.19 (1H, m), 2.32˜2.50 (6H, m), 2.66˜2.75 (11H, m), 3.06˜3.15 (2H, m), 3.19˜3.23 (11H, m), 3.32˜3.46 (1H, m), 3.88 (1H, s), 6.12˜6.13 (1H, d), 6.17 (1H, s), 6.50 (1H, s), 7.29-7.32 (1, d), 7.93 (1H, s), 7.99˜8.00 (1H, s), 8.08˜8.10 (1H, d), 8.18 (1H, s), 9.62 (1H, s).

### Procedure for the Preparation of Compound I

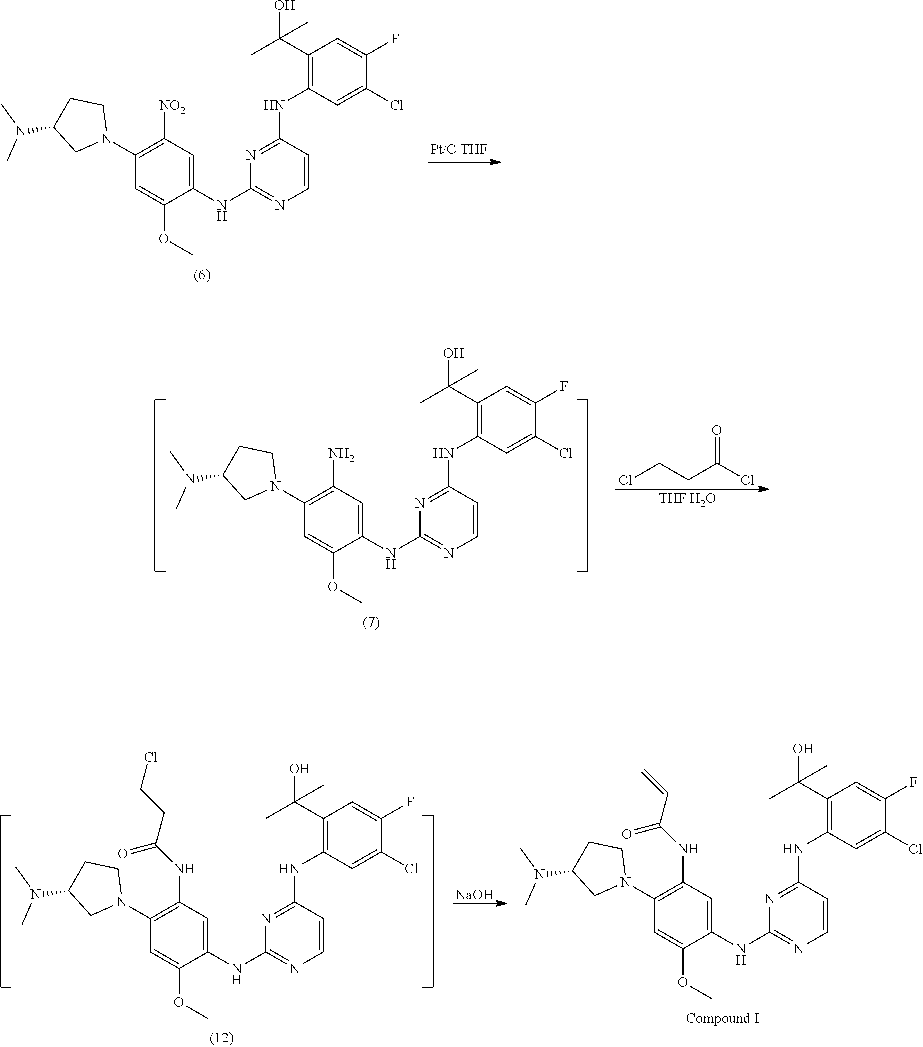

Compound (6) (86.0 kg) and THF (855.4 kg) with 5% Pt/C (3.3 kg, dry) were charged into a reactor. The hydrogen pressure of the reaction system was adjusted to 0.550˜0.688 MPa (0.5˜0.7 MPa), the temperature of the reaction system was adjusted to 50° C. (45˜55° C.). And the reaction system was stirred for 24h (20˜30h) until reaction was deemed complete. The temperature was adjusted to −10° C. (−15˜0° C.). The solution of compound (7) was filtered to another reactor. The cake was rinsed with THF (386 kg).

Purified water (176 kg) was charged into reactor, 3-chloropropionyl chloride (20.2 kg) was charged dropwise in THF (416 kg) at −15˜0° C. not less than 2 h. The mixture was stirred for 3h (2˜4h) at −15˜0° C. until reaction was deemed complete. The temperature was adjusted to 20° C. (15˜25° C.), a solution of 3.5% wt sodium hydroxide (709 kg) was charged dropwise at 20° C. (15˜25° C.) and stirred for 4h (3˜6h) until reaction was deemed complete. The resulting mixture was held for 1 h and the aqueous layer was separated. The organic phase was washed with 20% NaCl aq. solution (592 kg) twice. The organic solution was filtered by silica gel (235 kg), and the silica gel pad was washed with THF (2695 kg). The solution was concentrated to 340˜350 L and swapped with acetone (1008 kg) twice in total. Acetone (448 kg) was charged into the system and IPC sample was taken to control residual THF≤5.0%.

Purified water (95 kg) was charged into reactor, the temperature was adjusted to 56° C. (52˜59° C.) and the reaction system was stirred for 2h to a clear solution. Purified water (103 kg) was charged in 3 h and the solution was cooled to 40˜44° C. Seed (68 g) was charged into the solution and stirred for 14˜18h. Purified water (1118 kg) was charged dropwise at 40˜44° C. in a constant flow rate. The mixture was stirred for 2˜6 h at 40˜44° C., adjusted to 15˜25° C. in 4 h and stirred for 4h (2˜6h) then filtered and dried to obtain 76.71 kg solid Compound I in 84.5% yield with 99.63% HPLC purity by 98.9% assay. ¹H NMR (DMSO-d₆, 400 MHz), 1.49 (6H, s), 1.70˜1.71 (1H, m), 2.08 (1H, m), 2.15 (6H, s), 2.64˜2.68 (1H, m), 3.14˜3.19 (3H, m), 3.32˜3.34 (1H, m), 3.78 (3H, s), 5.65˜5.68 (1H, m), 6.04˜6.06 (1H, d), 6.13˜6.18 (2H, m), 6.45˜6.52 (2H, m), 7.27˜7.30 (1H, d), 7.59 (1H, s), 7.81 (1H, s), 7.94˜7.96 (1H, d), 8.13˜8.15 (1H, d), 9.24 (1H, s), 9.57 (1H, s).

### Re-Crystallization of Compound I

Crude Compound I (56.3 kg), and acetone/purified water ((743.2 kg, 9/1(v/v)) were added into a reactor. The reactor was heated to 48° C.˜55° C. to get a clear solution, and purified water (190 kg) was added into the reactor in 1-3h. The temperature was adjusted to 38° C.˜42° C. Seed (0.3 kg) was charged at 38° C.˜42° C. and stirred for 16 h. Purified water (997 kg) was charged dropwise into reactor at 38° C.˜42° C. in 6-8h and stirred for 4 h. The reaction system was adjusted to 20-25° C. in 3 h and stirred for 4 h. Then the reaction system was filtered and washed by acetone/purified water (103 kg, 2v/3v) twice. The wet cake was dried at 45° C. for 20h. 54.58 kg of Compound I was obtained with 99.83% HPLC purity, 96.7% isolated yield by 99.8% assay. ¹H NMR (DMSO-d₆, 400 MHz), 1.50 (6H, s), 1.71 (1H, m), 2.07 (1H, m), 2.15 (6H, s), 2.66 (1H, m), 3.14 (1H), 3.19 (2H), 3.38 (1H, m), 3.79 (3H, s), 5.67 (1H, dd, J=10.0, 2.0 Hz), 6.06 (1H, d, J=5.6 Hz), 6.15 (1H), 6.20 (1H), 6.50 (1H), 6.52 (1H), 7.29 (1H, d, J=10.8 Hz), 7.61 (1H, s), 7.85 (1H, s), 7.96 (1H, d, J=5.6 Hz), 8.16 (1H, d, J=7.6 Hz), 9.27 (1H, s), 9.60 (1H, s).

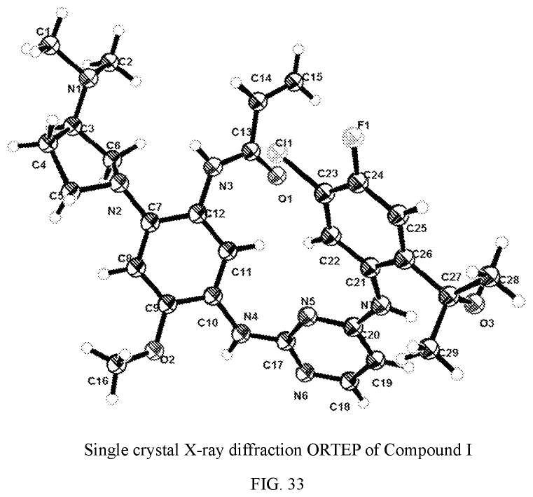

The single crystal X-ray diffraction ORTEP for compound I was shown in FIG. 33.

### Example 4: Preparation of Single Crystal of Compound I

Compound 21 (6 mg) was added to 1.5 mL of MOH in a 3 mL glass vial to give an unsaturated solution. Single crystal that was suitable for X-ray diffraction was successfully obtained by slowly evaporating the unsaturated solution at room temperature for three days.

### Crystal Data

| | |
|---|---|
| C₂₉H₃₅ClFN₇O₃ | F(000) = 2464 |
| Mᵣ = 584.09 | Dₓ = 1.299 Mg m⁻³ |
| Monoclinic, C2 | Cu Kα radiation, λ = 1.54178 Å |
| a = 33.0329 (10) Å | Cell parameters from 8990 reflections |
| b = 10.2716 (3) Å | θ = 2.5-66.3° |
| c = 19.7051 (5) Å | μ = 1.54 mm⁻¹ |
| β = 116.697 (1)° | T = 173 K |
| V = 5973.2 (3) Å³ | Block, colourless |
| Z = 8 | 0.09 × 0.05 × 0.03 mm |

### Data Collection

| | |
|---|---|
| Bruker APEX-II CCD diffractometer | 5918 reflections |
| | with I > 2σ(I) |
| ϕ and ω scans | Rᵢₙₜ = 0.119 |
| Absorption correction: multi-scan | θₘₐₓ = 66.6°, |
| SADABS2016/2 (Bruker, 2016/2) was used for | θₘᵢₙ = 2.5° |
| absorption correction. wR2(int) was 0.1427 before | |
| and 0.0851 after correction. The Ratio of | |
| minimum to maximum transmission is 0.6640. | |
| The λ/2 correction factor is Not present. | |
| Tₘᵢₙ = 0.211, Tₘₐₓ = 0.318 | h = −39→39 |
| 45085 measured reflections | k = −11→11 |
| 9712 independent reflections | l = −23→23 |

### Refinement

| | |
|---|---|
| Refinement on F² | Hydrogen site location: inferred from neighbouring sites |
| Least-squares matrix: full | H-atom parameters constrained |
| R[F² > 2σ(F²)] = 0.056 | w = 1/[σ2(Fₒ²) + (0.0685P)²] where P = (Fₒ² + 2F_{c}²)/3 |
| wR(F²) = 0.151 | (Δ/σ)ₘₐₓ < 0.001 |
| S = 1.02 | Δ [symbol] ₘₐₓ = 0.34 e Å⁻³ |
| 9712 reflections | Δ [symbol] ₘᵢₙ = −0.35 e Å⁻³ |
| 769 parameters | Absolute structure: Flack x determined using 1733 |
| | quotients [(I+) − (I−)]/[(I+) + (I−)] (Parsons, Flack and |
| | Wagner, Acta Cryst. B69 (2013) 249-259). |
| 664 restraints | Flack parameter: 0.022 (15) |

### Form A and Form B Inter-Conversion Study

Competitive slurry experiments were conducted to evaluate the relative stability of Form A and Form B. Inter-conversion study was performed at room temperature and 50° C. using single and binary solvents listed in the Table 1 below. About 80 mg (100 mg for 50° C.) of Form A and Form B (1:1, w/w) was weighed into sample vials (8 mL) and then 3 mL (2 mL for 50° C.) of solvents were added to each vial. The obtained suspensions were kept stirring for 7 days at room temperature and 50° C., then filtered at specified time. The wet and dried solids were analyzed by XRPD (dried in vacuum oven at 45° C.).

TABLE 1
Slurry in single or binary solvents.

| No | 1 | 2 | 3 | 4 | 5 | 6 | 7 |
|---|---|---|---|---|---|---|---|
| Solvent | Water | EtOH/ | EtOH/ | 1% water/ | EtOH/IPA | 30% Water/ | Acetone |
| (v/v) | | water(1/1) | IPA(1/1) | acetone | (7/2) | acetone | (no water) |

Results were summarized in Table 2 and Table 3. Form B was dominant in tested solvent systems with water activity below 0.15 at room temperature (24˜27° C.). Form B was more stable than Form A at 50° C. in all the tested solvent systems except pure water. Pure Form A and Form B were stable during drying process (50° C., vacuum) for at least 2 days. For a mixture of Form A and Form B, Form A was converted into Form B during the drying process.

TABLE 2
XRPD results of slurry in single or binary solvent at 24~27° C.

| | | Slurry at 24~27° C. | | | | | |
|---|---|---|---|---|---|---|---|
| | | Before dry | | | After dry | | |
| No | Solvent | 1 d | 3 d | 7 d | 1 d | 3 d | 7 d |
| 1 | Water | A + B | A + B | A + B | A + B | A + B | / |
| 2 | EtOH/water (1/1) | A + B | / | A + B | A + B | / | / |
| 3 | 30% water/acetone | A + B | / | A + B | A + B | / | / |
| 4 | 1% water/acetone | A + B | A + B | A + B(increased) | A + B | A + B | / |
| 5 | Acetone(no water) | A + B | A + B | A + B(increased) | A + B | A + B | / |
| 6 | EtOH/IPA (1/1) | A + B | / | A + B(increased) | A + B | / | / |
| 7 | EtOH/IPA (7/2) | A + B | / | A + B(increased) | A + B | / | |

TABLE 3
XRPD results of slurry in single or binary solvent at 50° C.

| | | Slurry at 50° C. | | | | | |
|---|---|---|---|---|---|---|---|
| | | Before dry | | | After dry | | |
| No | Solvent | 1 d | 3 d | 7 d | 1 d | 3 d | 7 d |
| 1 | Water | A + B | A + B | A + B | A + B | A + B | / |
| 2 | EtOH/water (1/1) | B | / | / | / | / | / |
| 3 | 30% water/acetone | A + B | B | / | A + B | / | / |
| 4 | 1% water/acetone | B | / | / | / | / | / |
| 5 | Acetone (no water) | A (minor) + B | B | / | B | B | / |
| 6 | EtOH/IPA (1/1) | B | / | / | / | / | / |
| 7 | EtOH/IPA (7/2) | B | / | / | / | / | / |

### Water Activity Study Form a and Form B

To study the effects of water on the stability of Form A and Form B, slurry experiments in the systems with different water activity were carried out. The results were listed in Table 5 and Table 6. Form A or Form B was suspended separately in different water activity systems (Table 4). About 20 mg Form A or Form B was suspended in 3 mL mixture of acetone and water at room temperature for 7 days. Residual solids were filtered and then dried in the vacuum oven at 45° C. for 8-24 hours. Dry solids were characterized by XRPD.

TABLE 4
Slurry in different water activity system.

| No | 1 | 2 | 3 | 4 | 5 | 6 |
|---|---|---|---|---|---|---|
| Condition(v/v) | 0% water/ | 1% water/ | 3% water/ | 11% water/ | 30% water/ | water |
| | acetone | acetone | acetone | acetone | acetone | |
| Water activity % | 0 | 0.154 | 0.312 | 0.548 | 0.747 | / |

TABLE 5
XRPD results of Form A slurry in different water activity solvent.

| | | Water | | | |
|---|---|---|---|---|---|
| | | activity % | | | |
| | | Water | 24~27° C. (before dry) | | |
| No | Condition | activity % | 1 d | 3 d | 7 d |
| 1 | 0% water/acetone | 0 (no water) | Form A | Form A | Form A + B |
| 2 | 1% water/acetone | 0.154 | / | / | / |
| 3 | 3% water/acetone | 0.312 | / | Form A | Form A + B |
| 4 | 11% water/acetone | 0.548 | / | / | / |
| 5 | 30% water/acetone | 0.747 | Form A | Form A | Form A + B |
| 6 | water | / | / | / | Form A |

TABLE 6
XRPD results of Form B slurry in different water activity solvent.

| | | Water | | | |
|---|---|---|---|---|---|
| | | activity % | | | |
| | | Water | 24~27° C. (before dry) | | |
| No | Condition | activity % | 1 d | 3 d | 7 d |
| 1 | 0% water/acetone | 0 (no water) | Form B | Form B | Form B |
| 2 | 1% water/acetone | 0.154 | / | / | / |
| 3 | 3% water/acetone | 0.312 | / | Form B | Form B |
| 4 | 11% water/acetone | 0.548 | / | / | / |
| 5 | 30% water/acetone | 0.747 | Form B | Form B | Form B |
| 6 | water | / | / | / | Form B |

### Stability in Storage for Form B

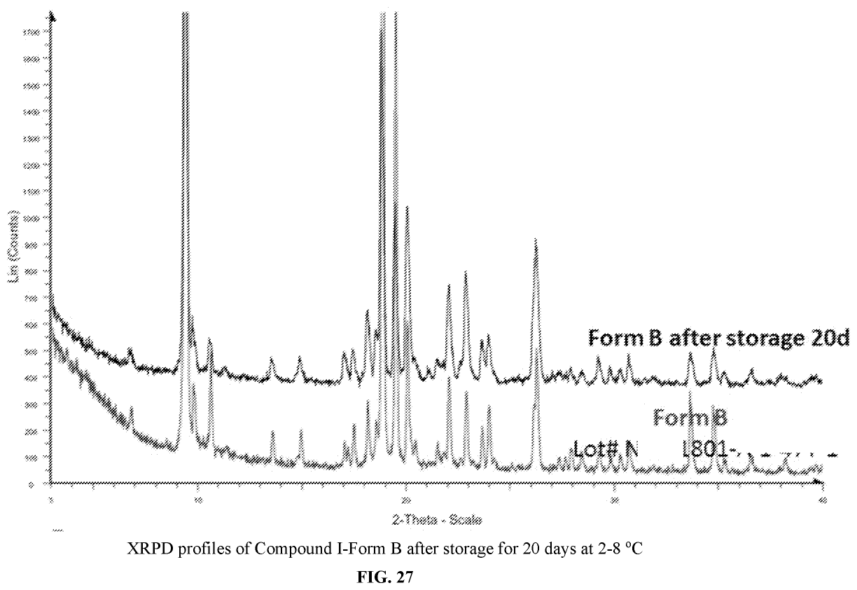

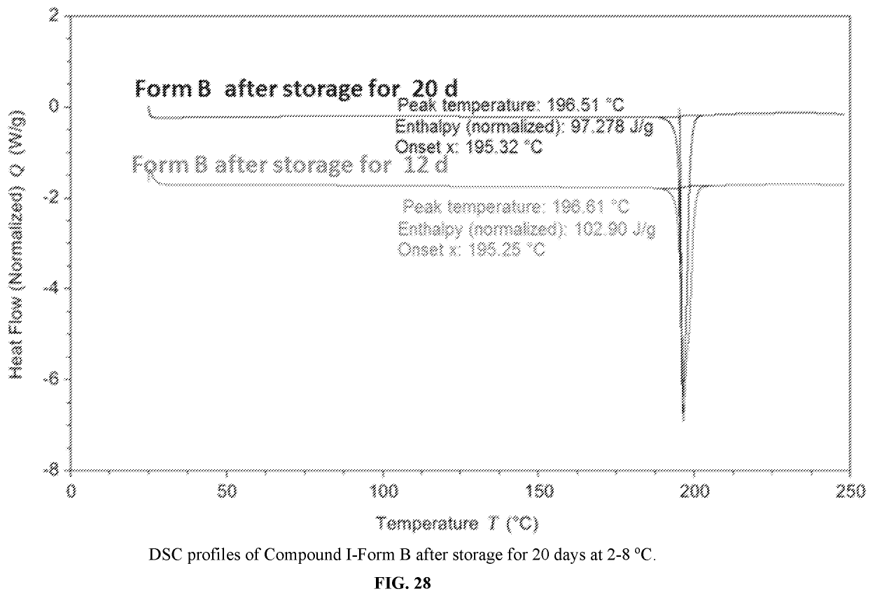

XRPD (FIG. 27) and DSC profile (FIG. 28) showed there were no form change observed for Form B after stored in at 2-8° C. for at least 20 d.

### Milling Study of Form B

Grinding and jet milling were carried out. About 10 mg Form B was added into mortar and grinded by pestle for 1 min and 2 min. Jet milling of Form B in 500 mg scale was performed. Samples before and after grinding and milling were analyzed by XRPD.
Instrument: Jet mill (Equipment number: PPD-OAJ-1)
Feeding speed: Manual
Feeding pressure: 0.3˜0.6 MPa
Milling One Pressure: 0.4˜0.8 MPa
Milling Two Pressure: 0.4˜0.8 MPa

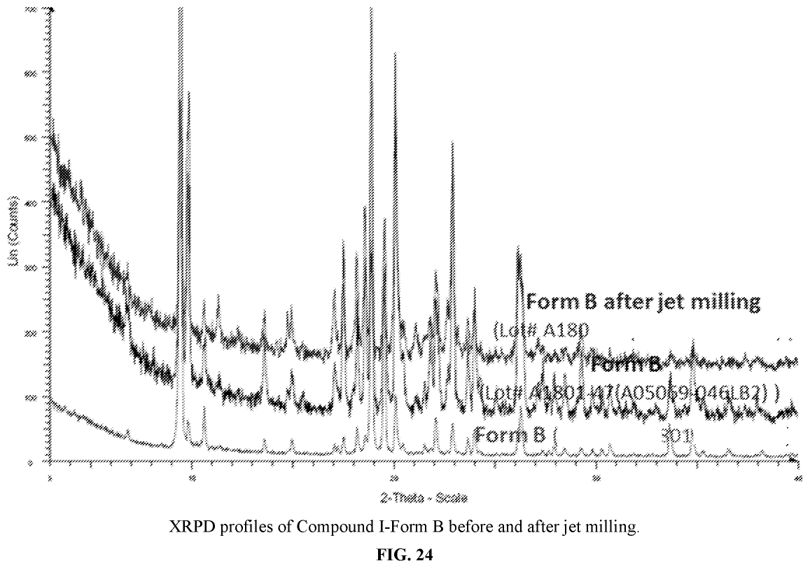

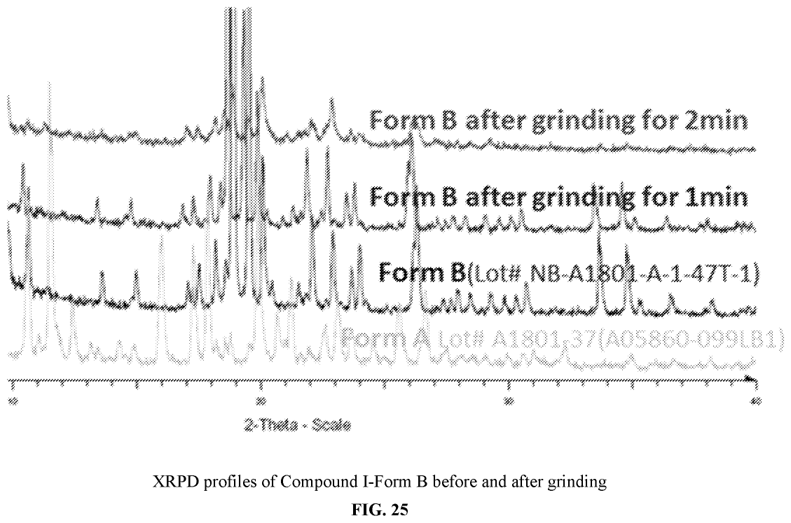

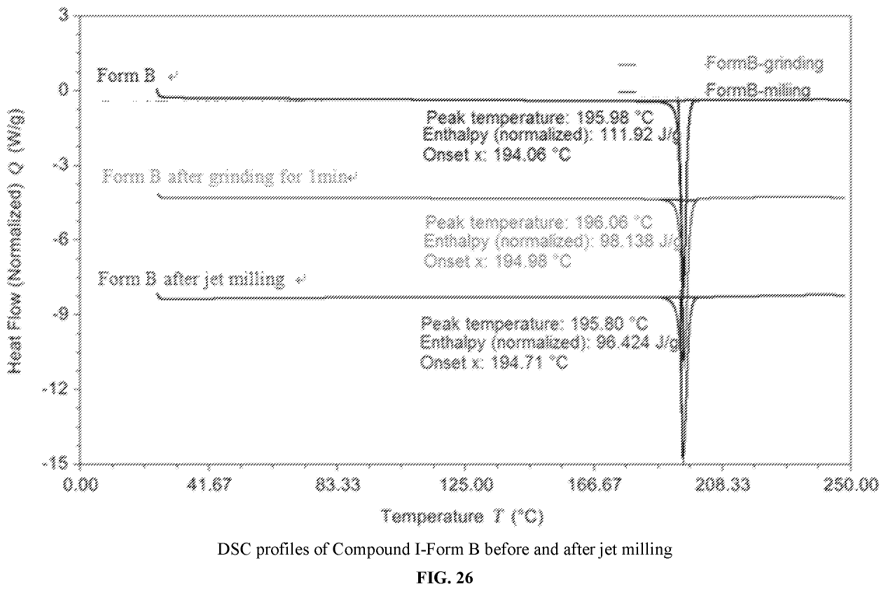

Grinding and jet milling were carried out to test physical stability of Form B in the milling process. As shown in XRPD results of obtained solids after grinding (FIG. 25), jet milling (FIG. 24), and DSC profile after jet milling (FIG. 26), the crystallinity decreased after grinding, but the crystal form of Form B was unchanged after milling and grinding.

### Preparation of Polymorphic Forms

### Procedures for the Preparation of Crystalline Form-A of the Free Base Compound I

Compound I free base crude (30 g) was dissolved into ethanol (210 mL), isopropanol (60 mL) and water (13 mL) at 70-75° C. to get a clear solution. The temperature was adjusted to 60-65° C., then Compound I-Form A crystal seed (0.06 g, 0.2% w/w) was added and stirred for at least 1 h at 60-65° C. The mixture was cooled to 50-55° C. and stirred for 2-3h, then cooled to 10-20° C. and filtered. The wet cake was washed with the mixture of ethanol/isopropanol. The wet cake was dried at 40-50° C. for at least 24h to give crystalline Compound I-Form A (13.6 g, 4500 yield).

The XRPD data for crystalline Form-A of the free base Compound I is shown in FIG. 1 and in Table 7.

TABLE 7
XRPD data for crystalline Compound I- Form A

| Peak No. | Angle (°2θ) | Rel. Intensity (%) |
|---|---|---|
| 1. | 5.949 | 18.7 |
| 2. | 9.178 | 8.0 |
| 3. | 9.450 | 5.1 |
| 4. | 9.819 | 1.5 |
| 5. | 10.676 | 35.6 |
| 6. | 11.108 | 26.0 |
| 7. | 11.617 | 61.6 |
| 8. | 12.484 | 78.7 |
| 9. | 13.215 | 20.1 |
| 10. | 13.495 | 6.8 |
| 11. | 14.435 | 8.6 |
| 12. | 14.614 | 7.6 |
| 13. | 14.963 | 10.5 |
| 14. | 16.019 | 44.6 |
| 15. | 16.483 | 9.1 |
| 16. | 17.344 | 60.0 |
| 17. | 17.939 | 100.0 |
| 18. | 18.286 | 25.3 |
| 19. | 18.852 | 23.3 |
| 20. | 19.593 | 23.6 |
| 21. | 20.042 | 87.0 |
| 22. | 20.794 | 36.0 |
| 23. | 21.337 | 26.4 |
| 24. | 22.012 | 16.7 |
| 25. | 22.629 | 22.9 |
| 26. | 23.224 | 24.7 |
| 27. | 23.711 | 41.9 |
| 28. | 24.638 | 32.5 |
| 29. | 25.031 | 9.2 |
| 30. | 25.714 | 21.6 |
| 31. | 26.769 | 23.7 |
| 32. | 27.602 | 13.1 |
| 33. | 28.207 | 4.9 |
| 34. | 30.045 | 5.8 |
| 35. | 32.424 | 7.1 |
| 36. | 33.643 | 4.0 |

The DSC data for crystalline Form-A of the free base Compound I is shown in FIG. 2. The DSC profile for crystalline Form-A of the free base Compound I shows an endothermic transition with an onset temperature of about 178.63° C. with a peak temperature of about 179.64° C., an associated enthalpy of 104.20 J/g.

The TGA data for crystalline Form-A of the free base Compound I is shown in FIG. 3. The TGA profile of Compound I-maleic acid salt shows a weight loss of about 0.232% before temperature reaching 160.00° C.

The DVS data for crystalline Form-A of the free base Compound I is shown in FIG. 4.

### Procedures for the Preparation of Crystalline Form-B of the Free Base Compound I

### Method 1.

Compound I-Form A (4 g) and ethanol (40 mL) were charged to reactor and keep stirring. The mixture was heated to 70° C. and stirred until the solid was totally dissolved. The solution was cooled to 60° C. at the rate of 0.1° C./min. Compound I-Form B seeds (0.02 g, 0.5% w/w) were added into solution. The solution was kept at 60° C. for 70˜80 min for seeds breeding. The suspension was cooled to 5° C. at 0.1° C./min and kept at 5° C. overnight. The suspension was filtered, and filter cake was dried in the oven for 5 h (50° C., vacuum) to give crystalline Compound I-Form B (˜80% yield)

### Method 2.

API crude (15 kg) was dissolved in acetone/purified water (258 L, 9/1, v/v) at 48-55° C. to a clear solution. Water (51 L) was charged at 48-55° C. The mixture was adjusted to 38-42° C. within 1 h. Compound I-Form B crystal seed (0.08 kg, 0.005, w/w) was charged at 38-42° C., and stirred for at least 14h. Water (268 L) was charged dropwise at 38-42° C. and stirred for at least 2 h. The mixture was cooled to 20-25° C. and stirred for at least 2 h. The mixture was filtered, and the cake was washed with the mixture of acetone/purified water. The wet cake was dried at 45-50° C. for at least 16h to give to give crystalline Compound I-Form B (14.1 kg, 94% yield)

The XRPD data for crystalline Form-B of the free base Compound I is shown in FIG. 5 and in Table 8.

TABLE 8
XRPD data for crystalline Compound I-Form B

| Peak No. | Angle (°2θ) | Rel. Intensity (%) |
|---|---|---|
| 1. | 6.785 | 1.4 |
| 2. | 9.388 | 100.0 |
| 3. | 9.761 | 3.2 |
| 4. | 10.594 | 4.9 |
| 5. | 11.326 | 0.9 |
| 6. | 13.581 | 3.0 |
| 7. | 14.775 | 1.1 |
| 8. | 14.951 | 2.9 |
| 9. | 17.045 | 3.3 |
| 10. | 17.478 | 3.9 |
| 11. | 18.158 | 9.7 |
| 12. | 18.564 | 4.5 |
| 13. | 18.861 | 56.3 |
| 14. | 19.500 | 27.0 |
| 15. | 20.061 | 24.5 |
| 16. | 20.448 | 2.2 |
| 17. | 21.091 | 0.7 |
| 18. | 21.503 | 3.2 |
| 19. | 22.070 | 12.6 |
| 20. | 22.911 | 10.9 |
| 21. | 23.683 | 5.4 |
| 22. | 24.002 | 5.0 |
| 23. | 26.296 | 16.4 |
| 24. | 27.363 | 1.3 |
| 25. | 27.691 | 1.0 |
| 26. | 27.962 | 1.8 |
| 27. | 28.482 | 1.2 |
| 28. | 29.285 | 2.5 |
| 29. | 29.830 | 1.4 |
| 30. | 30.331 | 1.9 |
| 31. | 30.728 | 3.5 |
| 32. | 31.935 | 0.7 |
| 33. | 33.710 | 4.5 |
| 34. | 340814 | 5.8 |
| 35. | 35.330 | 1.5 |
| 36. | 36.611 | 1.2 |
| 37. | 38.250 | 0.9 |

The DSC data for crystalline Form-B of the free base Compound I is shown in FIG. 6. The DSC profile for crystalline Form-A of the free base Compound I shows an endothermic transition with an onset temperature of about 194.84° C. with a peak temperature of about 195.74° C., an associated enthalpy of 111.60 J/g.

The TGA data for crystalline Form-B of the free base Compound I is shown in FIG. 7. The TGA profile of Compound I-maleic acid salt shows a weight loss of about 0.166% before temperature reaching 177.60° C.

The DVS data for crystalline Form-B of the free base Compound I is shown in FIG. 8.

### Physical Properties of Form A and Form B

TABLE 9
Physical Characterization Results

| Polymorph | Form A | Form B |
|---|---|---|
| Crystallinity | High | High |
| Particle shape & Size | Needle, ~10 μm | Needle, ~15 μm |
| Melting Point (DSC, ° C.) | Onset: 177.81° C.; | Onset: 194.78° C.; |
| | Peak: 179.74° C. | Peak: 195.82° C. |
| Enthalpy (DSC, J/g) | 112.0 J/g | 109.6 J/g |
| Weight loss (TGA, %) | 0.95% (<210° C.) | 0.47% (<210° C.) |
| DVS (ΔW %, 80% RH); | 0.63% | 0.04% |

TABLE 10
Solubility (S) in Organic Solvents and Water

| | Form A | Form B |
|---|---|---|
| Solvent | Solubility (mg/ml, r.t.) | Solubility (mg/ml, r.t.) |
| MeOH | 24.75 < S < 49.5 | 24.00 < S |
| EtOH | 13.37 < S < 17.83 | 10.40 < S < 13.87 |
| Isopropanol | 4.45 < S < 4.94 | S ~ 4.677 |
| Acetonitrile | S < 2.14 | S < 0.97 |
| Acetone | 5.12 < S < 5.69 | S < 5.88 |
| MTBE | S < 1.82 | S < 1.57 |
| Ethyl acetate | S < 2.16 | S < 1.06 |
| THF | 17.16 < S < 25.75 | 12.70 < S |
| water | S < 2.04 | S < 2.73 |

TABLE 11
Solubility Results in Bio-relevant Media

| | | Solubility (mg/mL) | | | pH value |
|---|---|---|---|---|---|
| Sample | Media | 0.5 h | 2 h | 24 h | at 24 h |
| Form A | SGF | 23.12 | 24.35 | 24.41 | — |
| | FaSSIF | 0.17 | 0.19 | 0.26 | — |
| | FeSSIF | 4.60 | 6.88 | 11.64 | — |
| | SIF | 0.029 | 0.04 | 0.04 | — |
| Form B | SGF | 23.51 | 24.20 | 25.78 | 5.15 |
| | FaSSIF | 0.15 | 0.16 | 0.24 | 6.43 |
| | FeSSIF | 3.18 | 2.29 | 11.24 | 5.50 |
| | SIF | 0.018 | 0.025 | 0.52 | 6.67 |

### In Vitro Cell Based Assay of Form A and Form B

TABLE 12
Form A and B in vitro cell based assay

| | | | | | Anti- | |
|---|---|---|---|---|---|---|
| | | | PD | | proliferation | |
| | | | IC₅₀ (nM) | | GI₅₀ (nM) | |
| | | | Form | Form | Form | Form |
| Gene | Mutation | Cell line | A | B | A | B |
| EGFR | 19Del | PC-9 | 2.13 | 2.29 | 4.76 | 4.23 |
| | L858R/T790M | NCI-H1975 | 2.10 | 2.07 | 2.22 | 1.45 |
| | H773_V774insNPH | Ba/F3_NPH | 32.44 | 35.16 | 58.18 | 66.27 |
| | Wildtype | A431 | 43.11 | 54.12 | 42.21 | 45.91 |
| HER2 | A775_G776insYVMA | 3T3_YVMA | 3.84 | 3.26 | 44.28 | 44.54 |

### Rat and Dog PK Study of Form A and Form B

TABLE 13
PK study of Form A and Form B in Rat

| | Form A | Form B | Form A | Form B |
|---|---|---|---|---|
| | (25 mg/kg) | (25 mg/kg | (75 mg/kg) | (75 mg/kg) |
| Mean t_{1/2} (h) | 4.4 | 4.1 | 6.0 | 5.8 |
| Median Tₘₐₓ (h) | 4.0 | 8.0 | 4.0 | 4.0 |
| Mean Cₘₐₓ (ng/ml) | 199 | 299 | 506 | 672 |
| Mean AUC_{0~48} (ng · h/mL) | 2131 | 2678 | 6872 | 8433 |

TABLE 14
PK study of Form A and Form B in Dog

| | Form A | Form B |
|---|---|---|
| | AUC_{0-24 h} | AUC_{0-24 h} |
| Dog PK | (hr*μM/ | (hr*μM/ |
| (10 mg/kg) | (mg/kg) | (mg/kg) |
| Male | 3.47 | 2.88 |
| Male | 5.47 | 5.29 |
| Female | 2.30 | 3.14 |
| Female | 1.80 | 3.75 |

### Example 5: Preparations of Pharmaceutical Salts and Salts Screening of Compound I

General Procedures for the Preparation of Compound I with Different Acids

50 mg Compound I were weighed into 2 mL vial, and then 900 uL acetone were added into the vial. Counter ion acid (1.1 e.q.) diluted (X10 times) in acetone was added to the vial. The vial was placed on the thermo-mixer and heated to 50° C. for 18 hrs, the vial was then cooled to 25° C. After keeping at 25° C. for 1 hr, the solid in suspension was isolated by centrifugation and dried in the vacuum oven at 30° C. for 3 hrs. The dried solid was characterized by XRPD. The dried solid obtained from above was subjected to re-slurry in isopropanol at 25° C. for 72 hrs. The solid in suspension was isolated by centrifugation and dried in the vacuum oven at 30° C. overnight. The dried solid was again characterized by XRPD, TGA and DSC.

TABLE 15
Salts screening of Compound I in different
solvents with different acids.

| Solvent | Acid | XRPD results |
|---|---|---|
| methanol | hydrochloric acid | amorphous |
| methanol | Methanesulfonic acid | amorphous |
| methanol | Sulfuric acid | amorphous |
| methanol | Phosphoric acid | amorphous |
| methanol | Maleic acid | amorphous |
| methanol | Fumaric acid | amorphous |
| methanol | Citric acid | amorphous |
| methanol | succinic acid | amorphous |
| methanol | L-malic acid | amorphous |
| methanol | L-(+)-tartaric acid | amorphous |
| acetone | hydrochloric acid | crystalline (scale-up) |
| acetone | Methanesulfonic acid | not available |
| acetone | Sulfuric acid | not available |
| acetone | Phosphoric acid | amorphous |
| acetone | Maleic acid | amorphous |
| acetone | Fumaric acid | amorphous |
| acetone | Citric acid | amorphous |
| acetone | succinic acid | amorphous |
| acetone | L-malic acid | amorphous |
| acetone | L-(+)-tartaric acid | crystalline |
| acetonitrile | hydrochloric acid | amorphous |
| acetonitrile | Methanesulfonic acid | amorphous |
| acetonitrile | Sulfuric acid | amorphous |
| acetonitrile | Phosphoric acid | amorphous |
| acetonitrile | Maleic acid | amorphous |
| acetonitrile | Fumaric acid | crystalline |
| acetonitrile | Citric acid | amorphous |
| acetonitrile | succinic acid | amorphous |
| acetonitrile | L-malic acid | amorphous |
| acetonitrile | L-(+)-tartaric acid | amorphous |
| ethyl acetate | hydrochloric acid | amorphous |
| ethyl acetate | Methanesulfonic acid | amorphous |
| ethyl acetate | Sulfuric acid | amorphous; crystalline |
| | | (reslurry in POH) |
| ethyl acetate | Phosphoric acid | amorphous & crystalline |
| ethyl acetate | Maleic acid | amorphous; crystalline |
| | | (reslurry in ⁱPOH) |
| ethyl acetate | Fumaric acid | amorphous |
| ethyl acetate | Citric acid | amorphous |
| ethyl acetate | succinic acid | amorphous |
| ethyl acetate | L-malic acid | amorphous |
| ethyl acetate | L-(+)-tartaric acid | crystalline |

### 5.1: Preparation of (R)—N-(5-((4-((5-chloro-4-fluoro-2-(2-hydroxypropan-2-yl)phenyl)amino)pyrimidin-2-yl)amino)-2-(3-(dimethylamino) pyrrolidin-1-yl)-4-methoxyphenyl)acrylamide Hydrochloride (Compound I-hydrochloric acid salt)

### Hydrochloric Acid Salt (Preparation in Acetone Solution)

1800 mg Compound I were suspended into 20.0 mL acetone at 60° C. Hold at 60° C. for 1 hr. 1.1 e.q. Hydrochloric acid of acetone solution (6.76 mL, 0.5 mol/L) was added into the suspension dropwise. The suspension was held at 60° C. for 3 hrs. Then the suspension was cooled to 25° C. and held at 25° C. for 20 hrs. The suspension was filtered by funnel and the wet cake was washed by 0.5 mL acetone. Wet solids were dried in the vacuum oven at 30° C. for 72 hrs. Dried off-white solids (1623.3 mg, 83.4% yield) were obtained. The dried solid was characterized by XRPD, TGA, DSC, and DVS. Salt ratio of hydrochloride to Compound I was determined by IC-test. The measured chloride content was 5.78%, compared to 5.72%-theoretical content of chloride in a 1:1 salt ratio.

The XRPD data for crystalline form of the Compound I-hydrochloric acid salt is shown in FIG. 13 and in Table 16.

TABLE 16
XRPD data for crystalline form of the
Compound I-hydrochloric acid salt

| Peak No. | Angle (°2θ) | Rel. Intensity (%) |
|---|---|---|
| 1. | 7.304 | 20.0 |
| 2. | 7.808 | 17.0 |
| 3. | 9.052 | 31.6 |
| 4. | 9.350 | 54.5 |
| 5. | 10.443 | 30.7 |
| 6. | 13.624 | 8.1 |
| 7. | 14.854 | 23.8 |
| 8. | 16.111 | 4.6 |
| 9. | 16.532 | 5.6 |
| 10. | 17.209 | 43.2 |
| 11. | 17.610 | 17.4 |
| 12. | 18.212 | 100.0 |
| 13. | 18.771 | 14.8 |
| 14. | 19.537 | 36.3 |
| 15. | 19.789 | 49.5 |
| 16. | 20.137 | 11.9 |
| 17. | 20.912 | 40.2 |
| 18. | 21.167 | 90.9 |
| 19. | 21.510 | 42.3 |
| 20. | 21.907 | 14.0 |
| 21. | 22.874 | 901 |
| 22. | 23.253 | 24.5 |
| 23. | 23.596 | 18.4 |
| 24. | 25.239 | 14.3 |
| 25. | 25.792 | 10.4 |
| 26. | 26.242 | 32.4 |
| 27. | 26.760 | 10.6 |
| 28. | 27.221 | 21.8 |
| 29. | 27.426 | 25.6 |
| 30. | 28.223 | 9.8 |
| 31. | 29.682 | 21.7 |
| 32. | 30.637 | 26.2 |
| 33. | 31.120 | 13.5 |
| 34. | 32.212 | 4.8 |
| 35. | 33.192 | 6.5 |
| 36. | 34.006 | 5.2 |
| 37. | 34.486 | 7.2 |
| 38. | 37.955 | 5.8 |
| 39. | 39.088 | 5.3 |

The TGA data for crystalline form of the Compound I-hydrochloric acid salt is shown in FIG. 20. The TGA profile of Compound I-hydrochloric acid salt shows a weight loss of about 0.759% before temperature reaching 175° C.

The DSC data for crystalline form of the Compound I-hydrochloric acid salt is shown in FIG. 20. The DSC profile for crystalline form of the Compound I-hydrochloric acid salt shows an endothermic transition with an onset temperature of about 207.77° C. with a peak temperature of about 212.14° C., an associated enthalpy of 75.60 J/g.

The DVS data for crystalline form of the Compound I-hydrochloric acid salt is shown in FIG. 23.

### 5.2: Preparation of (R)—N-(5-((4-((5-chloro-4-fluoro-2-(2-hydroxypropan-2-yl)phenyl)amino)pyrimidin-2-yl)amino)-2-(3-(dimethylamino) pyrrolidin-1-yl)-4-methoxyphenyl)acrylamide L-(+)-tartrate (Compound I-L-(+)-tartaric acid salt, pattern I)

50 mg Compound I were weighed into 2 mL vial, and then 900 uL acetone were added into the vial. L-(+)-tartaric acid (1.1 e.q.) diluted (X10 times) in acetone was added to the vial. The vial was placed on the thermo-mixer and heated to 50° C. for 18 hrs, the vial was then cooled to 25° C. After keeping at 25° C. for 1 hr, the solid in suspension was isolated by centrifugation and dried in the vacuum oven at 30° C. for 3 hrs. The dried solid was characterized by XRPD. The dried solid obtained from above was subjected to re-slurry in isopropanol at 25° C. for 72 hrs. The solid in suspension was isolated by centrifugation and dried in the vacuum oven at 30° C. overnight. The dried solid was again characterized by XRPD, TGA and DSC.

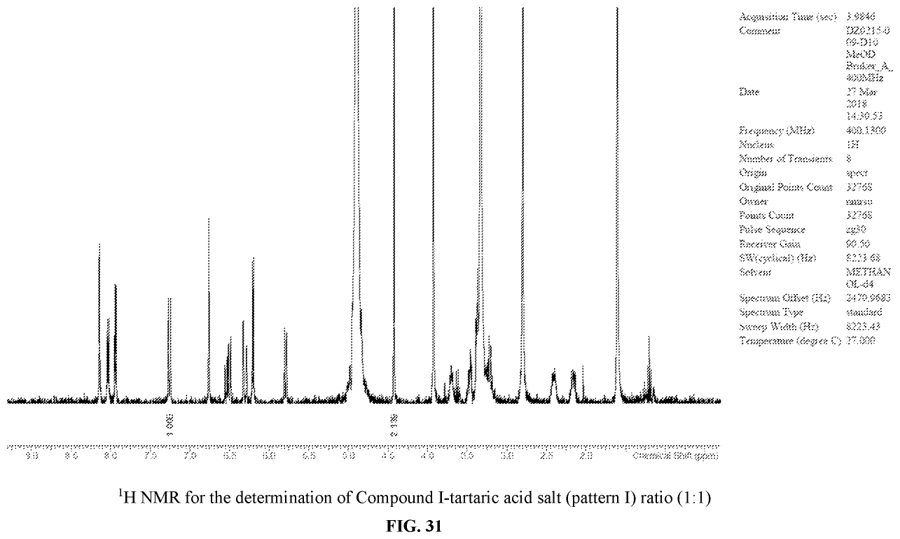

The ¹H-NMR data for crystalline form of the Compound I-L-(+)-tartaric acid salt (pattern I) is shown in FIG. 31.

The XRPD data for crystalline form of the Compound I-L-(+)-tartaric acid salt (pattern I) is shown in FIG. 9 and in Table 17.

TABLE 17
XRPD data for crystalline form of the Compound
I-L-(+)-tartaric acid salt (pattern I)

| Peak No. | Angle (°2θ) | Rel. Intensity (%) |
|---|---|---|
| 1. | 5.341 | 85.8 |
| 2. | 5.382 | 73.7 |
| 3. | 7.291 | 4.2 |
| 4. | 10.501 | 71.0 |
| 5. | 10.922 | 100.0 |
| 6. | 11.835 | 7.9 |
| 7. | 14.397 | 3.5 |
| 8. | 15.047 | 7.5 |
| 9. | 16.373 | 19.7 |
| 10. | 17.858 | 13.7 |
| 11. | 18.520 | 12.3 |
| 12. | 18.989 | 7.6 |
| 13. | 22.021 | 5.1 |
| 14. | 23.962 | 4.3 |

The TGA data for crystalline form of the Compound I-L-(+)-tartaric acid salt (pattern I) is shown in FIG. 15. The TGA profile of Compound I-L-(+)-tartaric acid salt (pattern I) shows a weight loss of about 2.52% before temperature reaching 100° C.

The DSC data for crystalline form of the Compound I-L-(+)-tartaric acid salt (pattern I) is shown in FIG. 15.

The DSC profile for crystalline form of the (+)-L-tartaric acid salt of Compound I (pattern I, prepared in acetone) shows the first endothermic transition with an onset temperature of about 36.91° C. with a peak temperature of about 56.29° C., an associated enthalpy of 44.43 J/g and The second endothermic transition with an onset temperature of about 136.73° C. with a peak temperature of about 140.18° C., an associated enthalpy of 19.53 J/g.

### 5.3: Preparation of (R)—N-(5-((4-((5-chloro-4-fluoro-2-(2-hydroxypropan-2-yl)phenyl)amino)pyrimidin-2-yl)amino)-2-(3-(dimethylamino) pyrrolidin-1-yl)-4-methoxyphenyl)acrylamide fumarate (Compound I-fumaric acid salt

### Fumaric Acid Salt (Preparation in Acetone Solution)

1800 mg Compound I were suspended into 25.0 mL acetone at 60° C. Hold at 60° C. for 1 hr. Fumaric acid solids (392.3 mg, 1.1 e.q.) was added into the suspension. The suspension was held at 60° C. for 3 hrs. Then the suspension was cooled to 25° C. and held at 25° C. for 20 hrs. About 5 mg seed was added into the solution. The solution was held at 25° C. for 42 hrs. The suspension was filtered by funnel and the wet cake was washed by 0.5 mL acetone. Wet solids were dried in the vacuum oven at 30° C. for 72 hrs. Dried light-yellow solids (1588.8 mg, 71.7% yield) were obtained. The dried solid was characterized by XRPD, TGA, DSC, and DVS.

### Fumaric Acid Salt (Preparation in Ethanol Solution)

300 mg Compound I were suspended into 5.0 mL acetone at 60° C. The suspension was held at 60° C. for 1 hr. 1.1 e.q. fumaric acid of ethanol solution (1.7 mL, 0.33 mol/L) was added into the suspension dropwise. The suspension was held at 60° C. for 3 hrs. Then the suspension was cooled to 25° C. and held at 25° C. for 20 hrs. The suspension was filtered by funnel and the wet cake was washed by 0.5 mL ethanol. Wet solids were dried in the vacuum oven at 30° C. for 72 hrs. Dried off-white solids (284.7 mg, 76.2% yield) were obtained as solids. Salt ratio determined by ¹H-NMR=1.0:1.0 (Compound I: fumaric acid). The dried solid was characterized by XRPD, TGA, DSC, and DVS.

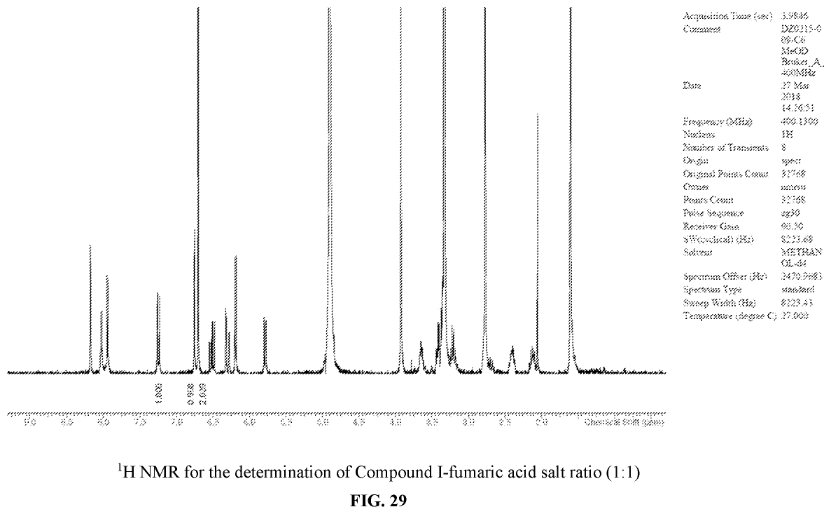

The ¹H-NMR data for crystalline form of the Compound I-fumaric acid salt is shown in FIG. 29.

The XRPD data for crystalline of the Compound I-fumaric acid salt is shown in FIG. 10 and in Table 18.

TABLE 18
XRPD data for crystalline form of
the Compound I-fumaric acid salt

| Peak No. | Angle (°2θ) | Rel. Intensity (%) |
|---|---|---|
| 1. | 5.162 | 10.4 |
| 2. | 7.902 | 7.6 |
| 3. | 8.900 | 4.2 |
| 4. | 10.355 | 5.5 |
| 5. | 11.053 | 6.3 |
| 6. | 11.632 | 24.4 |
| 7. | 11.919 | 51.2 |
| 8. | 12.326 | 32.8 |
| 9. | 13.080 | 36.4 |
| 10. | 13.706 | 100.0 |
| 11. | 14.413 | 8.2 |
| 12. | 14.951 | 21.3 |
| 13. | 15.793 | 35.1 |
| 14. | 16.583 | 22.0 |
| 15. | 17.228 | 32.0 |
| 16. | 17.788 | 4.2 |
| 17. | 18.518 | 23.5 |
| 18. | 18.856 | 32.9 |
| 19. | 19.540 | 50.3 |
| 20. | 20.152 | 60.7 |
| 21. | 20.626 | 34.9 |
| 22. | 21.014 | 9.9 |
| 23. | 21.529 | 6.1 |
| 24. | 21.871 | 10.3 |
| 25. | 22.138 | 46.5 |
| 26. | 23.255 | 16.2 |
| 27. | 23.792 | 20.6 |
| 28. | 24.210 | 57.4 |
| 29. | 25.006 | 8.3 |
| 30. | 26.572 | 16.0 |
| 31. | 26.910 | 15.1 |
| 32. | 27.536 | 10.0 |
| 33. | 28.651 | 10.4 |

The TGA data for crystalline form of the Compound I-fumaric acid salt is shown in FIG. 17. The TGA profile of Compound I-fumaric acid salt shows a weight loss of about 2.857% before temperature reaching 55° C. and a further weight loss of about 2.424% between temperature range 55-140′° C.

The DSC data for crystalline form of the Compound I-fumaric acid salt is shown in FIG. 17. The DSC profile for crystalline form of the Compound I-fumaric acid salt shows an endothermic transition with an onset temperature of about 48.86° C. with a peak temperature of about 68.34° C., an associated enthalpy of 28.63 J/g and a later endothermic transition with an onset temperature of about 132.79° C. with a peak temperature of about 141.78° C., an associated enthalpy of 27.78 J/g.

The DVS data for crystalline form of the Compound I-fumaric acid salt is shown in FIG. 22.

### 5.4: Preparation of (R)—N-(5-((4-((5-chloro-4-fluoro-2-(2-hydroxypropan-2-yl)phenyl)amino)pyrimidin-2-yl)amino)-2-(3-(dimethylamino) pyrrolidin-1-yl)-4-methoxyphenyl)acrylamide sulfate (Compound I-sulfuric acid salt)

50 mg Compound I were weighed into 2 mL vial, and then 900 uL acetone were added into the vial. Sulfuric acid (1.1 e.q.) diluted (X10 times) in acetone was added to the vial. The vial was placed on the thermo-mixer and heated to 50° C. for 18 hrs, the vial was then cooled to 25° C. After keeping at 25° C. for 1 hr, the solid in suspension was isolated by centrifugation and dried in the vacuum oven at 30° C. for 3 hrs. The dried solid was characterized by XRPD. The dried solid obtained from above was subjected to re-slurry in isopropanol at 25° C. for 72 hrs. The solid in suspension was isolated by centrifugation and dried in the vacuum oven at 30° C. overnight. The dried solid was again characterized by XRPD, TGA and DSC.

The XRPD data for crystalline form of the Compound I-sulfuric acid salt is shown in FIG. 11 and Table 19.

TABLE 19
XRPD data for crystalline form of
the Compound I-sulfuric acid salt

| Peak No. | Angle (°2θ) | Rel. Intensity (%) |
|---|---|---|
| 1. | 5.997 | 73.5 |
| 2. | 7.535 | 15.5 |
| 3. | 12.156 | 48.1 |
| 4. | 14.895 | 12.3 |
| 5. | 17.456 | 17.2 |
| 6. | 17.369 | 25.0 |
| 7. | 18.190 | 100.0 |
| 8. | 19.522 | 17.2 |
| 9. | 20.513 | 20.2 |
| 10. | 22.024 | 10.1 |
| 11. | 22.650 | 16.8 |
| 12. | 24.862 | 11.9 |
| 13. | 25.732 | 9.7 |

The TGA data for crystalline form of the Compound I-sulfuric acid salt is shown in FIG. 18. The TGA profile of Compound I-sulfuric acid salt shows a weight loss of about 4.85% before temperature reaching 120° C.

The DSC data for crystalline form of the Compound I-sulfuric acid salt is shown in FIG. 18. The DSC profile for crystalline form of the Compound I-sulfuric acid salt shows an endothermic transition with an onset temperature of about 181.24° C. with a peak temperature of about 195.93° C., an associated enthalpy of 11.87 J/g and a later endothermic transition with an onset temperature of about 210.59° C. with a peak temperature of about 226.02° C., an associated enthalpy of 26.76 J/g.

### 5.5: Preparation of (R)—N-(5-((4-((5-chloro-4-fluoro-2-(2-hydroxypropan-2-yl)phenyl)amino)pyrimidin-2-yl)amino)-2-(3-(dimethylamino)pyrrolidin-1-yl)-4-methoxyphenyl)acrylamide maleate (Compound I-maleic acid salt)

50 mg Compound I were weighed into 2 mL vial, and then 900 uL acetone were added into the vial. Maleic acid (1.1 e.q.) diluted (X10 times) in acetone was added to the vial. The vial was placed on the thermo-mixer and heated to 50° C. for 18 hrs, the vial was then cooled to 25° C. After keeping at 25° C. for 1 hr, the solid in suspension was isolated by centrifugation and dried in the vacuum oven at 30° C. for 3 hrs. The dried solid was characterized by XRPD. The dried solid obtained from above was subjected to re-slurry in isopropanol at 25° C. for 72 hrs. The solid in suspension was isolated by centrifugation and dried in the vacuum oven at 30° C. overnight. The dried solid was again characterized by XRPD, TGA and DSC.

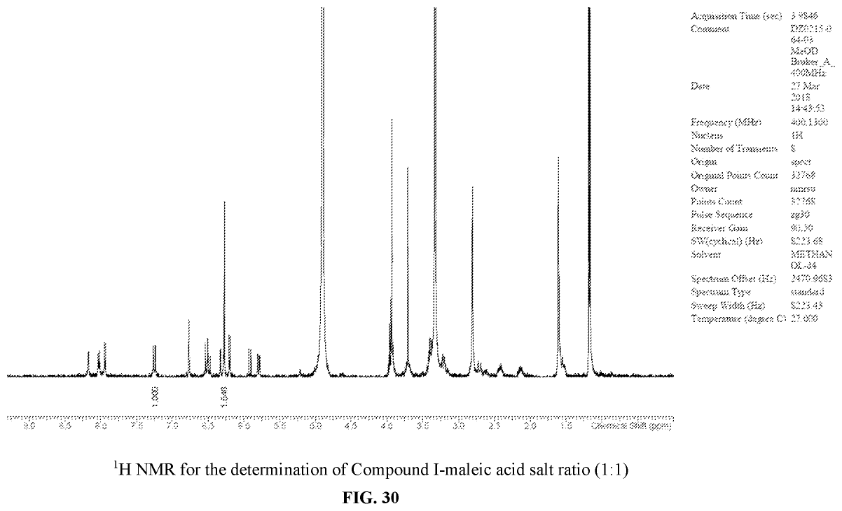

The ¹H-NMR data for crystalline form of the Compound I-maleic acid salt is shown in FIG. 30.

The XRPD data for crystalline form of the Compound I-maleic acid salt is shown in FIG. 12 and in Table 20.

TABLE 20
XRPD data for crystalline form of the Compound I-maleic acid salt

| Peak No. | Angle (°2θ) | Rel. Intensity (%) |
|---|---|---|
| 1. | 4.904 | 32.2 |
| 2. | 7.452 | 29.8 |
| 3. | 9.573 | 20.1 |
| 4. | 11.939 | 38.1 |
| 5. | 12.740 | 21.0 |
| 6. | 13.194 | 11.3 |
| 7. | 15.638 | 60.5 |
| 8. | 16.104 | 100.0 |
| 9. | 18.457 | 21.8 |
| 10. | 20.979 | 37.6 |
| 11. | 22.651 | 38.9 |
| 12. | 24.274 | 28.5 |
| 13. | 25.669 | 25.7 |

The TGA data for crystalline form of the Compound I-maleic acid salt is shown in FIG. 19. The TGA profile of Compound I-maleic acid salt shows a weight loss of about 5.25% before temperature reaching 100° C.

The DSC data for crystalline form of the Compound I-maleic acid salt is shown in FIG. 19.

### 5.6: Preparation of (R)—N-(5-((4-((5-chloro-4-fluoro-2-(2-hydroxypropan-2-yl)phenyl)amino)pyrimidin-2-yl)amino)-2-(3-(dimethylamino) pyrrolidin-1-yl)-4-methoxyphenyl)acrylamide tartrate (Compound I-(+)-L-tartaric acid salt, pattern II)

### L-(+)-Tartaric Acid Salt (Pattern II, Preparation in Ethanol Solution)

300 mg Compound I were suspended into 5.0 mL acetone at 60° C. The suspension was held at 60° C. for 1 hr. 1.1 e.q. L-(+)-Tartaric acid of ethanol solution (1.10 mL, 0.5 mol/L) was added into the suspension dropwise. The suspension was held at 60° C. for 3 hrs. Then the suspension was cooled to 25° C. and held at 25° C. for 20 hrs. The suspension was filtered by funnel and the wet cake was washed by 0.5 mL ethanol. Wet solids were dried in the vacuum oven at 30° C. for 72 hrs. Dried off-white solids (303.0 mg, 78.0% yield) were obtained. Salt ratio determined by ¹H-NMR=1.0:1.0 (Compound I: L-(+)-Tartaric acid salt). The dried solid was characterized by XRPD, TGA, DSC, and DVS.

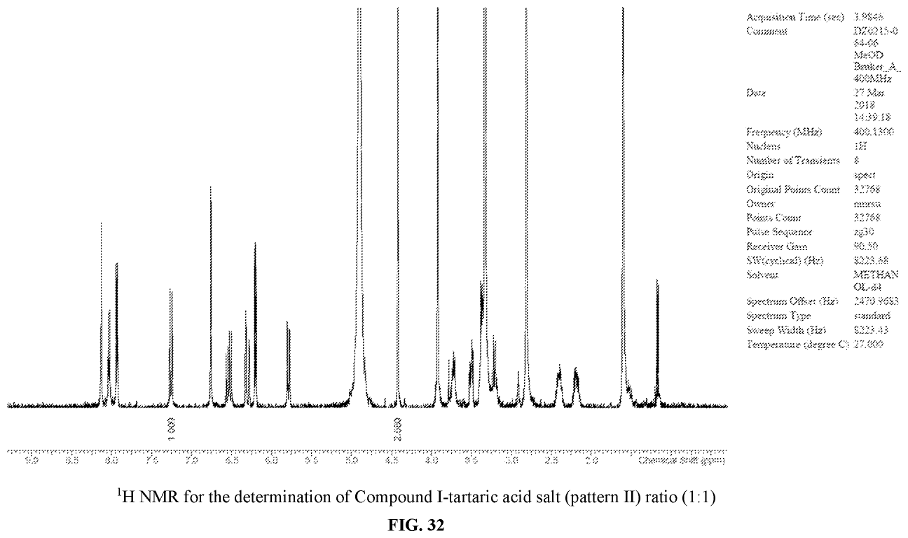

The ¹H-NMR data for crystalline form of the Compound I-L-(+)-tartaric acid salt (pattern II) is shown in FIG. 32.

The XRPD data for crystalline form of the Compound I-L-(+)-tartaric acid salt (pattern II) is shown in FIG. 14 and Table 21.

TABLE 21
XRPD data for crystalline form of the Compound I-L-(+)-tartaric
acid salt (pattern II, preparation in ethanol solution)

| Peak No. | Angle (°2θ) | Rel. Intensity (%) |
|---|---|---|
| 1. | 5.349 | 81.0 |
| 2. | 7.293 | 44.4 |
| 3. | 7.954 | 7.3 |
| 4. | 10.024 | 26.8 |
| 5. | 10.600 | 100.0 |
| 6. | 11.886 | 28.0 |
| 7. | 12.696 | 16.1 |
| 8. | 13.762 | 12.1 |
| 9. | 14.558 | 20.3 |
| 10. | 15.242 | 14.6 |
| 11. | 15.906 | 8.8 |
| 12. | 16.804 | 14.0 |
| 13. | 18.033 | 38.5 |
| 14. | 18.979 | 37.8 |
| 15. | 19.890 | 37.4 |
| 16. | 20.928 | 18.9 |
| 17. | 21.263 | 25.6 |
| 18. | 22.146 | 29.7 |
| 19. | 22.824 | 15.0 |
| 20. | 23.438 | 10.2 |
| 21. | 24.024 | 24.5 |
| 22. | 25.545 | 7.3 |
| 23. | 27.043 | 6.5 |
| 24. | 28.498 | 6.8 |
| 25. | 29.987 | 12.9 |

The TGA data for crystalline form of the Compound I-L-(+)-tartaric acid salt (pattern II) is shown in FIG. 16. The TGA profile of Compound I-L-(+)-tartaric acid salt (pattern II) shows a weight loss of about 3.587% before temperature reaching 100° C.

The DSC data for crystalline form of the Compound I-L-(+)-tartaric acid salt (pattern II) is shown in FIG. 16. The DSC profile for crystalline form of the Compound I-L-(+)-tartaric acid salt (pattern II) shows an endothermic transition with an onset temperature of about 64.62° C. with a peak temperature of about 75.67° C., an associated enthalpy of 34.23 J/g and a later endothermic transition with an onset temperature of about 137.25° C. with a peak temperature of about 140.39° C., an associated enthalpy of 17.53 J/g.

### Solubility of Different Compound I Salts in Bio-Relevant Media and pH Values

TABLE 22
Solubility of different Compound I salts
in bio-relevant media and pH values

| | Hydrochloric acid salt | | Fumaric acid salt | | Tartaric acid salt | |
|---|---|---|---|---|---|---|
| | Solubility | | Solubility | | Solubility | |
| | (mg/mL, | pH | (mg/mL, | pH | (mg/mL, | pH |
| Media | 24 hrs) | value | 24 hrs) | value | 24 hrs) | value |
| SGF | 11.85 | 5.65 | >12.15 | 3.98 | >9.0 | 3.42 |
| FaSSIF | 1.95 | 6.62 | >10.05 | 5.22 | >9.1 | 4.57 |
| FeSSIF | >11.05 | 5.44 | >9.75 | 4.94 | >9.25 | 4.76 |

### Example 6: Dissolution of Compound I, Freebase, Form B, Tablets (200 mg)

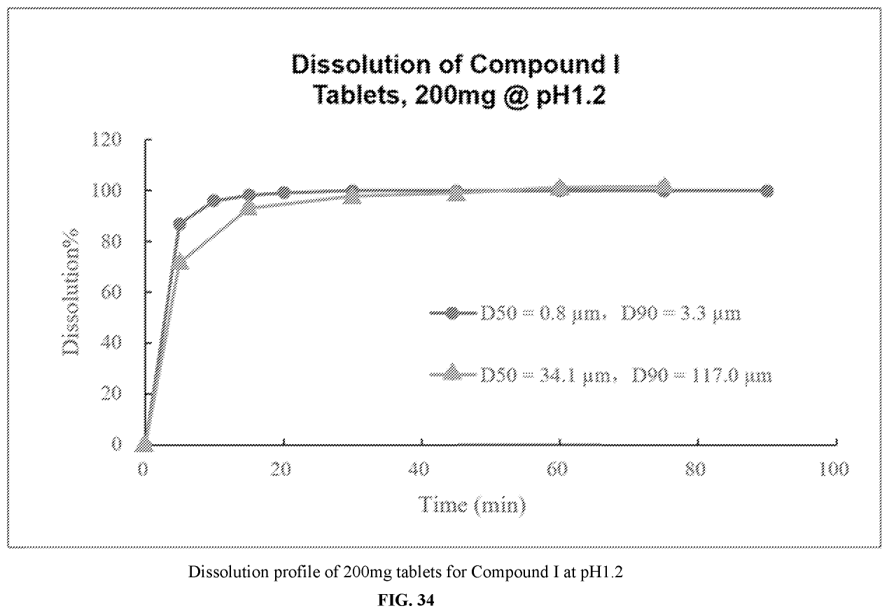

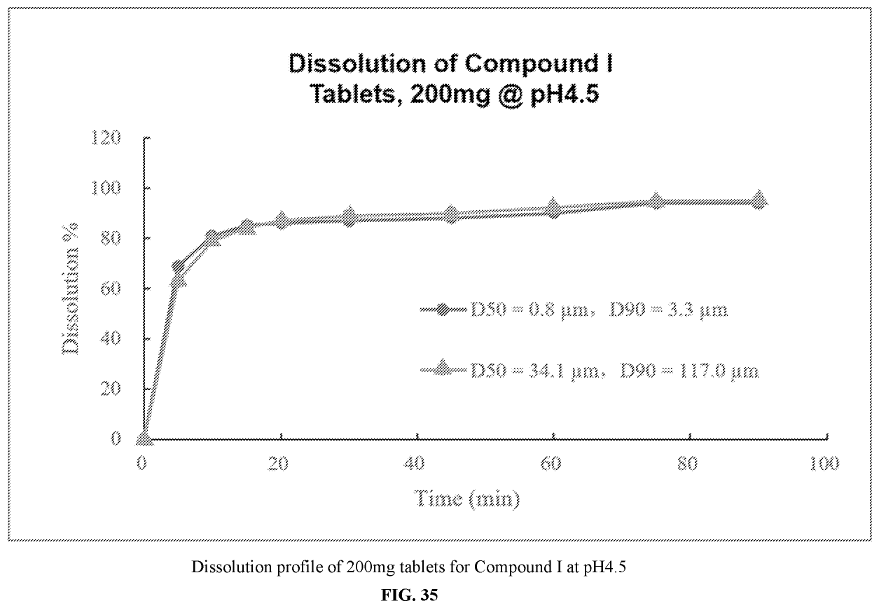

Compound I (freebase, form B) tablets were manufactured using both milled (D50=0.8 μm and D₉₀=3.3 μm) and un-milled (D₅₀=34.1 μm and D₉₀=117.0 μm) conditions. The dissolution profiles at different pH (1.2 and 4.5) are summarized in FIG. 34 and FIG. 35. At pH1.2, greater than 85% Compound I was released in 15 min. It can be seen that the polymorphs of the present disclosure exhibit enhanced rates of dissolution

The Tablets were Manufactured in Accordance with the Following Manufacturing Process:

The formulation included diluents, binders, disintegrants, lubricants, glidants and coating materials. The preferred excipients were microcrystalline cellulose lactose, Lactose Monohydrate, Croscarmellose Sodium, Hydroxypropyl Cellulose, Colloidal Silicon Dioxide and Magnesium Stearate. Coating material was Opadry®. The content of Microcrystalline Cellulose was 10%-70%, preferably 20%-38%; the content of Lactose Monohydrate was 15%-75%, preferably 25%-40%, the content of Croscarmellose Sodium is 1%-18%, preferably 2%-10%; the content of Hydroxypropyl Cellulose was 1%-15%, preferably 2%-8%; the content of Magnesium Stearate and Colloidal Silicon Dioxide was 0.25%-5%, preferably 0.5%-3%; the coating weight gain was 1.5%-8%, preferably 2%-5%.

### Tablets Manufacturing

Weighed the formula amount of Compound I (freebase, form B), lactose monohydrate, colloidal silicon dioxide, Microcrystalline cellulose, Microcrystalline cellulose, croscarmellose sodium, hydroxypropyl cellulose and magnesium stearate. The hydroxypropyl cellulose, microcrystalline cellulose and colloidal silicon dioxide were passed through a screen together. Compound I, lactose monohydrate, croscarmellose sodium, microcrystalline cellulose and magnesium stearate were passed through a screen. The screened excipients, except for the extra-granular phases (microcrystalline cellulose, croscarmellose sodium and magnesium stearate), were charged into a high shear wet granulator bowl and blend. The purified water was sprayed onto the blended powders. If necessary, additional purified water was sprayed. The wet materials was continued to granulate after spraying. The wet materials were charged through a screen. The materials from above were charged into a fluid bed and dried, the drying process was monitored by loss-on-drying. The dried granule was charged through a screen. The milled granule, extra croscarmellose sodium and microcrystalline cellulose were charged into the bin blender and blended. Then magnesium stearate was charged into the bin blender. The lubricated blend was compressed into tablets.

### Tablets Coating

A 12% (w/w) Opadry® suspension was prepared. the core tablets were preheated until the exhaust temperature reaches about 40-50° C. and then coating was started. Coating solution was sprayed until the coating weight gain reached the target range. Heating was stopped after spraying has finished, then the coated tablets were dried and discharged.

TABLE 23
Composition of Compound I (freebase, form B) Tablets

| | Formulation | | | |
|---|---|---|---|---|
| | percent | mg/Tablet | | |
| composition | % | 150 mg | 200 mg | Function |

Intra-granular

| | | | | |
|---|---|---|---|---|
| Compound I | 25.00 | 150.00 | 200.00 | Ingredient |
| Lactose Monohydrate | 32.00 | 192.00 | 256.00 | Diluent |
| Microcrystalline Cellulose | 24.50 | 147.00 | 196.00 | Diluent |
| Colloidal Silicon Dioxide | 1.00 | 6.00 | 8.00 | Glidant |
| Croscarmellose Sodium | 2.00 | 12.00 | 16.00 | Disintegrant |
| Hydroxypropyl Cellulose | 4.50 | 27.00 | 36.00 | Binder |
| Water | N/A | q.s. | q.s. | Solvent |

Extra-granular

| | | | | |
|---|---|---|---|---|
| Microcrystalline Cellulose | 8.00 | 48.00 | 64.00 | Diluent |
| Croscarmellose Sodium | 2.00 | 12.00 | 16.00 | Disintegrant |
| Magnesium Stearate | 1.00 | 6.00 | 8.00 | Lubricant |
| Total for Core Tablets | 100.00 | 600.00 | 800.00 | N/A |

Coating Material

| | | | | |
|---|---|---|---|---|
| Opadry ® | 3.00 | 18.00 | 24.00 | Coating Agent |
| Water | N/A | q.s. | q.s. | Solvent |
| Total | N/A | 618.00 | 824.00 | N/A |

1. A crystalline form of (R)—N-(5-((4-((5-chloro-4-fluoro-2-(2-hydroxypropan-2-yl)phenyl)amino)pyrimidin-2-yl)amino)-2-(3-(dimethylamino)pyrrolidin-1-yl)-4-methoxyphenyl) acrylamide (Compound I) or pharmaceutically acceptable salts thereof.

2.-137. (canceled)

138. The crystalline form of claim 1, which is Form A of Compound I having:
(a) an X-ray powder diffraction (XRPD) pattern comprising peaks at diffraction angles (2θ) of 11.62±0.20, 12.48±0.20, 17.34±0.20, and 20.04±0.20 degrees, or
(b) a XRPD pattern substantially as shown in FIG. 1, or
(c) a DSC thermogram comprising an endotherm with a desolvation onset at about 178.6° C. and a peak at about 179.6° C., or
(d) a TGA thermogram exhibiting a mass loss of about 0.23% upon heating from about 38° C. to about 160° C., or
(e) a TGA thermogram substantially similar to FIG. 3, or
(f) a DSC thermogram substantially similar to FIG. 2, or
(g) a DVS vapor sorption gram substantially similar to FIG. 4.

139. The crystalline form of claim 1, which is Form B of Compound I having:
(a) a XRPD pattern comprising peaks at 2θ of 9.39±0.20, 18.86±0.20, 19.50±0.20, and 20.06±0.20 degrees, or
(b) a XRPD pattern substantially as shown in FIG. 5, or
(c) a DSC thermogram comprising an endotherm with a desolvation onset at about 194.8° C. and a peak at about 195.7° C., or
(d) a TGA thermogram exhibiting a mass loss of less than 0.17% upon heating from about 38° C. to about 178° C., or
(e) a TGA thermogram substantially similar to FIG. 7, or
(f) a DSC thermogram substantially similar to FIG. 6, or
(g) a DVS vapor sorption gram substantially similar to FIG. 8.

140. The crystalline form of claim 1, which is a crystalline form of a pharmaceutically acceptable salt of Compound I, optionally, wherein the pharmaceutically acceptable salt is selected from hydrochloric acid salt, L-(+)-tartaric acid salt, fumaric acid salt, sulfuric acid salt, and maleic acid salt.

141. The crystalline form of claim 1, which is a crystalline form of Compound I hydrochloric acid salt having:
(a) a XRPD pattern comprising peaks at 2θ of 9.35±0.20, 17.21±0.20, 18.21±0.20, 19.79±0.20, and 21.17±0.20 degrees, or
(b) a XRPD pattern substantially as shown in FIG. 13, or
(c) a DSC thermogram comprising an endotherm with a desolvation onset at about 207.8° C. and a peak at about 212.1° C.
(d) a TGA thermogram exhibiting a mass loss of about 0.76% upon heating to about 175° C.
(e) a TGA/DSC thermogram substantially similar to FIG. 20
a DVS vapor sorption gram substantially similar to FIG. 23.

142. The crystalline form of claim 1, which is a crystalline form of Compound I L-(+)-tartaric acid salt.

143. The crystalline form of claim 142, which is Compound I L-(+)-tartaric acid salt pattern I having:
(a) a XRPD pattern comprising peaks at 2θ of 5.34±0.20, 5.38±0.20, 10.50±0.20, 10.92±0.20, and 16.37±0.20 degrees, or
(b) a XRPD pattern substantially as shown in FIG. 9, or
(c) a DSC thermogram comprising an endotherm with a desolvation onset at about 207.8° C. and a peak at about 212.1° C., or
(d) a TGA thermogram exhibiting a mass loss of about 0.76% upon heating to about 175° C., or
(e) a TGA/DSC thermogram substantially similar to FIG. 15.

144. The crystalline form of claim 142, which is Compound I L-(+)-tartaric acid salt pattern II having:
(a) a XRPD pattern comprising peaks at 2θ of 10.02±0.20, 18.03±0.20, 19.89±0.20, 21.15±0.20, and 21.26±0.20 degrees, or
(b) a XRPD pattern substantially as shown in FIG. 14, or
(c) a DSC thermogram comprising an endotherm with a desolvation onset at about 137.2° C. and a peak at about 140.4° C., or
(d) a TGA thermogram exhibiting a mass loss of about 3.59% upon heating to about 100° C., or
(e) a TGA/DSC thermogram substantially similar to FIG. 16, or
(f) a DVS vapor sorption gram substantially similar to FIG. 21.

145. The crystalline form of claim 1, which is a crystalline form of Compound I fumaric acid salt having:
(a) a XRPD pattern comprising peaks at 2θ of 11.92±0.20, 13.71±0.20, 19.54±0.20, 20.15±0.20, and 24.21±0.20 degrees, or
(b) a XRPD pattern substantially as shown in FIG. 10, or
(c) a DSC thermogram comprising an endotherm with a desolvation onset at about 48.9° C. and a peak at about 68.3° C., or
(d) a DSC thermogram further comprising an later endotherm with a desolvation onset at about 132.79° C. and a peak at about 141.78° C., or
(e) a TGA thermogram exhibiting a mass loss of about 2.86% upon heating to about 55° C., or
(f) a TGA thermogram exhibiting a mass loss of about 2.42% upon heating from about 55° C. to about 140° C., or
(g) a TGA/DSC thermogram substantially similar to FIG. 17, or
(h) a DVS vapor sorption gram substantially similar to FIG. 22.

146. The crystalline form of claim 1, which is a crystalline form of Compound I sulfuric acid salt having:
(a) a XRPD pattern comprising peaks at 2θ of 6.00±0.20, 12.16±0.20, 17.37±0.20, 18.19±0.20, and 20.51±0.20 degrees, or
(b) a XRPD pattern substantially as shown in FIG. 11, or
(c) a DSC thermogram comprising an endotherm with a desolvation onset at about 181.2° C. and a peak at about 195.9° C., or
(d) a DSC thermogram further comprising an endotherm with a later desolvation onset at about 210.6° C. and a peak at about 226.0° C., or
(e) a TGA thermogram exhibiting a mass loss of about 4.85% upon heating to about 120° C., or
(f) a TGA/DSC thermogram substantially similar to FIG. 18.

147. The crystalline form of claim 1, which is a crystalline form of Compound I maleic acid salt having:
(a) a XRPD pattern comprising peaks at 2θ of 11.94±0.20, 15.64±0.20, 16.10±0.20, 20.98±0.20, and 22.65±0.20 degrees, or
(b) a XRPD pattern substantially as shown in FIG. 12, or
(c) a DSC thermogram comprising an endotherm with a desolvation onset at about 64.6° C. and a peak at about 75.7° C., or
(d) a DSC thermogram further comprising an endotherm with a later desolvation onset at about 137.3° C. and a peak at about 140.4° C., or
(e) a TGA thermogram exhibiting a mass loss of about 3.59% upon heating to about 100° C., or
(f) a TGA/DSC thermogram substantially similar to FIG. 19.

148. The crystalline form of claim 1, wherein the crystalline form is substantially pure polymorphs.

149. A compound of Formula (I): 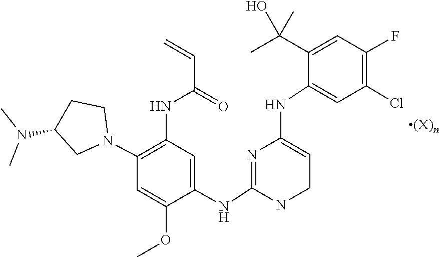
wherein,
n=1 or 2; and
X is hydrochloric acid, methanesulfonic acid, sulfuric acid, phosphoric acid, L-(+)-tartaric acid, fumaric acid, citric acid, succinic acid, L-malic acid or maleic acid.

150. A pharmaceutical composition comprising one or more crystalline forms according to claim 1, and a pharmaceutically acceptable carrier.

151. A method of inhibiting ErbB or BTK by using one or more crystalline form according to claim 1.

152. A method of treating an ErbB associated diseases or BTK associated diseases in a subject, comprising administering to the subject an effective amount of one or more crystalline form according to claim 1.

153. A crystalline form according to claim 1, in combination with a second therapeutic agent, preferably an anti-tumour agent.

154. Process for production of crystals of pharmaceutically acceptable salt of Compound I by dissolving Compound I in acetone or ethanol solution, adding corresponding acid in acetone or ethanol solution, and leaving the solution to crystallize and isolating the crystals of the pharmaceutically acceptable salt of Compound I, wherein the pharmaceutically acceptable salt is selected from hydrochloric acid salt, L-(+)-tartaric acid salt, fumaric acid salt, sulfuric acid salt, and maleic acid salt.
